(19) United States
(12) Patent Application Publication
(10) Pub. No.: US 2025/0090252 A1
(43) Pub. Date: Mar. 20, 2025

(54) METHOD AND DEVICE FOR GENERATING COORDINATE SYSTEM TRANSFORMATION INFORMATION, AND ROBOTIC SURGICAL SYSTEM INCLUDING THE SAME

(71) Applicant: LIVSMED INC., Seongnam-si (KR)

(72) Inventors: Soon Yong JEONG, Seongnam-si (KR); Jin Hyuk YOON, Seongnam-si (KR)

(21) Appl. No.: 18/884,503

(22) Filed: Sep. 13, 2024

(30) Foreign Application Priority Data
Sep. 14, 2023 (KR) 10-2023-0122648

(51) Int. Cl. A61B 34/32; B25J 9/16; B25J 19/02

(52) U.S. Cl. CPC A61B 34/32; B25J 9/1653; B25J 19/023

(57) ABSTRACT

A method according to an embodiment of the present disclosure includes calculating a first angle difference between a preset reference coordinate system and a first robot base coordinate system, based on a dial manipulation value of a user obtained from a first angle measurement dial mounted on a first robot, calculating a second angle difference between the preset reference coordinate system and a second robot base coordinate system, based on a dial manipulation value of the user obtained from a second angle measurement dial mounted on a second robot, and generating coordinate system transformation information between the first robot base coordinate system and the second robot base coordinate system, based on the first angle difference and the second angle difference.

### CROSS-REFERENCE TO RELATED APPLICATION

This application claims the benefit of Korean Patent Application Number 10-2023-0122648, filed on Sep. 14, 2023 with the Korean Intellectual Property Office (KIPO), the entire content of which is incorporated herein by reference.

### TECHNICAL FIELD

The present disclosure relates to a method and device for generating coordinate system transformation information, and a robotic surgical system including the same.

### BACKGROUND ART

In medical terms, surgery refers to the treatment of diseases by cutting, incising, or manipulating a skin, a mucous membrane, or other tissues by using medical devices. In particular, open surgery for incising and opening the skin of a surgical site to treat, shape, or remove an organ or the like therein causes issues such as bleeding, side effects, patient's pain, or scarring. Therefore, recently, surgery performed by forming a certain hole on a skin and inserting only a medical device, for example, a laparoscopic instrument or a surgical instrument, or surgery using a robot has been spotlighted as an alternative.

Here, a surgical robot refers to a robot that has a function of replacing a surgical action performed by a surgeon. Advantageously, the surgical robot may operate more accurately and precisely as compared with a human and enable remote surgery.

Meanwhile, a surgical robot is generally composed of a master robot and a slave robot. When an operator manipulates a control lever (e.g., a handle) provided on the master robot, a surgical tool coupled to or held by a robotic arm of the slave robot may be manipulated to perform surgery.

However, some problems may arise as an operator performs surgery by remotely manipulating a surgical tool through a surgical robot rather than physically manipulating the surgical tool manually. For example, a problem may arise in that, even though the operator manipulates the control lever provided on the master robot, the slave robot does not perform a motion desired by the operator due to mechanical constraints. In addition, as the operator manipulates the control lever provided on the master robot, there may be a difference between a movement of the slave robot intended by the operator and an actual movement of the slave robot.

The above-mentioned background art is technical information possessed by the inventor for the derivation of the present disclosure or acquired during the derivation of the present disclosure, and cannot necessarily be said to be a known technique disclosed to the general public prior to the filing of the present disclosure.

### DISCLOSURE

### Technical Problem

Some embodiments according to the present disclosure provide a method and device for generating coordinate system transformation information, and a robotic surgical system including the same. Technical objectives of the present disclosure are not limited to the foregoing, and other unmentioned objectives or advantages of the present disclosure would be understood from the following description and be more clearly understood from the embodiments of the present disclosure. In addition, it would be appreciated that the objectives and advantages of the present disclosure may be implemented by means provided in the claims and a combination thereof.

### Technical Solution

A first aspect of the present disclosure may provide a method, performed by a coordinate system transformation device in a robotic surgical system, of generating coordinate system transformation information, the method including: calculating a first angle difference between a preset reference coordinate system and a first robot base coordinate system, based on a first dial manipulation value of a user obtained from a first angle measurement dial mounted on a first robot; calculating a second angle difference between the preset reference coordinate system and a second robot base coordinate system, based on a second dial manipulation value of the user obtained from a second angle measurement dial mounted on a second robot; and generating coordinate system transformation information between the first robot base coordinate system and the second robot base coordinate system, based on the first angle difference and the second angle difference.

In the first aspect, the first angle measurement dial or the second angle measurement dial may be configured to receive physical dial manipulation by the user and obtain the first dial manipulation value corresponding to the first angle difference or the second dial manipulation value corresponding to the second angle difference.

In the first aspect, the calculating of the first angle difference includes, in a case in which the first angle measurement dial does not use the first robot base coordinate system, calculating a third angle difference between the preset reference coordinate system and a coordinate system used by the first angle measurement dial, based on the first dial manipulation value obtained from the first angle measurement dial.

In the first aspect, the calculating of the second angle difference may include, in a case in which the second angle measurement dial does not use the second robot base coordinate system, calculating a fourth angle difference between the preset reference coordinate system and a coordinate system used by the second angle measurement dial, based on the second dial manipulation value obtained from the second angle measurement dial.

In the first aspect, the first angle measurement dial may be mounted at a position of the first robot other than a position that is defined as a reference point of the first robot base coordinate system.

In the first aspect, the second angle measurement dial may be mounted at a position of the second robot other than a position that is defined as a reference point of the second robot base coordinate system.

In the first aspect, the coordinate system used by the first angle measurement dial may have a reference point that is the position of the first robot at which the first angle measurement dial is mounted.

In the first aspect, the coordinate system used by the second angle measurement dial may have a reference point that is the position of the second robot at which the second angle measurement dial is mounted.

In the first aspect, the method may further include calculating a fifth angle difference between a first angle measurement dial coordinate system and the first robot base coordinate system, based on a first angle measurement value obtained from a first angle measurement sensor mounted on a robotic arm of the first robot.

In the first aspect, the method may further include calculating a sixth angle difference between a second angle measurement dial coordinate system and the second robot base coordinate system, based on a second angle measurement value obtained from a second angle measurement sensor mounted on a robotic arm of the second robot.

In the first aspect, the generating of the coordinate system transformation information may include: generating first coordinate system transformation information between the first robot base coordinate system and the preset reference coordinate system by using the first angle difference; generating second coordinate system transformation information between the second robot base coordinate system and the preset reference coordinate system by using the second angle difference; and generating the coordinate system transformation information between the first robot base coordinate system and the second robot base coordinate system, based on the first coordinate system transformation information and the second coordinate system transformation information.

In the first aspect, the first angle difference or the second angle difference may refer to a difference in yaw value of a reference point of the first robot base coordinate system with respect to the preset reference coordinate system, or a difference in yaw value of a reference point of the second robot base coordinate system with respect to the preset reference coordinate system.

In the first aspect, the preset reference coordinate system may include any one of a coordinate system used by a surgical bed in an operating room where the first robot and the second robot are arranged, or a coordinate system used by a master robot included in the robotic surgical system.

In the first aspect, the generating of the coordinate system transformation information may include: calculating a relative angle between the first robot base coordinate system and the second robot base coordinate system by using the first angle difference and the second angle difference; and calculating a rotation transformation matrix between the first robot base coordinate system and the second robot base coordinate system, based on the relative angle.

In the first aspect, the method may further include, after surgery is terminated, initializing the coordinate system transformation information.

In the first aspect, the method may further include monitoring the coordinate system transformation information before surgery begins, and when the coordinate system transformation information is not generated or the monitored coordinate system transformation information corresponds to the initialized coordinate system transformation information, generating an angle measurement dial manipulation alarm signal.

A second aspect of the present disclosure may provide a device for generating coordinate system transformation information in a robotic surgical system, the device including: a non-transitory computer-readable medium configured to store at least one program; and a processor configured to execute the at least one program to calculate a first angle difference between a preset reference coordinate system and a first robot base coordinate system, based on a dial manipulation value of a user obtained from a first angle measurement dial mounted on a first robot, calculate a second angle difference between the preset reference coordinate system and a second robot base coordinate system, based on a dial manipulation value of the user obtained from a second angle measurement dial mounted on a second robot, and generate coordinate system transformation information between the first robot base coordinate system and the second robot base coordinate system, based on the first angle difference and the second angle difference.

In the second aspect, the preset reference coordinate system may include any one of a coordinate system used by a surgical bed in an operating room where the first robot and the second robot are arranged, or a coordinate system used by a master robot included in the robotic surgical system.

In the second aspect, the processor may be further configured to calculate a relative angle between the first robot base coordinate system and the second robot base coordinate system by using the first angle difference and the second angle difference, and calculate a rotation transformation matrix between the first robot base coordinate system and the second robot base coordinate system, based on the relative angle.

In a second aspect, the processor may be further configured to initialize the coordinate system transformation information after surgery is terminated.

In the second aspect, the processor may be further configured to monitor the coordinate system transformation information before surgery begins, and when the coordinate system transformation information is not generated or the monitored coordinate system transformation information corresponds to the initialized coordinate system transformation information, generate an angle measurement dial manipulation alarm signal.

A third aspect of the present disclosure may provide a non-transitory computer-readable recording medium having recorded thereon a program for causing a computer to execute the method according to the first aspect.

A fourth aspect of the present disclosure may provide a surgical robot including: one or more robotic arms configured to perform a motion by handle manipulation by an operator; a surgical instrument coupled to each of the one or more robotic arms; an angle measurement dial configured to receive physical manipulation of a dial by a user to obtain a dial manipulation value of the user; and a device to generate coordinate system transformation information based on an angle difference between coordinate systems, wherein the device is to calculate a first angle difference between a preset reference coordinate system and a first robot base coordinate system, based on a dial manipulation value of the user obtained from a first angle measurement dial mounted on a first robot, calculate a second angle difference between the preset reference coordinate system and a second robot base coordinate system, based on a dial manipulation value of the user obtained from a second angle measurement dial mounted on a second robot, and generate coordinate system transformation information between the first robot base coordinate system and the second robot base coordinate system, based on the first angle difference and the second angle difference.

A fifth aspect of the present disclosure may provide a laparoscopic camera robot including: one or more robotic arms configured to perform a motion by handle manipulation by an operator; a laparoscopic surgical camera coupled to each of the one or more robotic arms; an angle measurement dial configured to receive physical manipulation of a dial by a user to obtain a dial manipulation value of the user; and a device to generate coordinate system transformation information based on an angle difference between coordinate systems, wherein the device is to calculate a first angle difference between a preset reference coordinate system and a first robot base coordinate system, based on a dial manipulation value of the user obtained from a first angle measurement dial mounted on a first robot, calculate a second angle difference between the preset reference coordinate system and a second robot base coordinate system, based on a dial manipulation value of the user obtained from a second angle measurement dial mounted on a second robot, and generate coordinate system transformation information between the first robot base coordinate system and the second robot base coordinate system, based on the first angle difference and the second angle difference.

A sixth aspect of the present disclosure may provide a robotic surgical system including: a surgical robot including a first angle measurement dial configured to receive a first physical manipulation of the first angle measurement dial by a user to obtain a first dial manipulation value of the user; a laparoscopic camera robot including a second angle measurement dial configured to receive a second physical manipulation of the second angle measurement dial by the user to obtain a second dial manipulation value of the user; and a master robot including a device to generate coordinate system transformation information based on an angle difference between coordinate systems, wherein the device is to calculate a first angle difference between a preset reference coordinate system and a first robot base coordinate system, based on the first dial manipulation value of the user obtained from the first angle measurement dial mounted on the surgical robot, calculate a second angle difference between the preset reference coordinate system and a second robot base coordinate system, based on the second dial manipulation value of the user obtained from the second angle measurement dial mounted on the laparoscopic camera robot, and generate coordinate system transformation information between the first robot base coordinate system and the second robot base coordinate system, based on the first angle difference and the second angle difference.

In a sixth aspect, the surgical robot may have a sensor configured to measure a first angle measurement value between a first angle measurement dial coordinate system and the surgical robot base coordinate system.

In a sixth aspect, the laparoscopic camera robot may have a sensor configured to measure a second angle measurement value between a second angle measurement dial coordinate system and the laparoscopic camera robot base coordinate system.

In addition, other methods and systems for implementing the present disclosure, and a computer-readable recording medium having recorded thereon a computer program for executing the methods may be further provided.

### Advantageous Effects

According to the embodiments of the present disclosure described herein, coordinate system transformation information between robots included in a robotic surgical system may be generated with only intuitive and simple manipulation by a user. Also, for example, when the coordinate system transformation information, according to the embodiments of the present disclosure, is generated, and a movement in a surgical robot base coordinate system is transformed into a movement in a camera robot base coordinate system by using the coordinate system transformation information, manipulating the manipulation member of the master robot to the left moves the surgical instrument displayed on the display member of a master robot to the left. This allows more intuitive and accurate manipulation of the surgical robot by the user to perform surgery with the surgical robot.

Effects of the present disclosure are not limited to the foregoing, and other unmentioned effects would be clearly understood by those skilled in the art from the following description.

### DESCRIPTION OF DRAWINGS

FIG. 1 is a conceptual diagram illustrating a robotic surgical system according to an embodiment of the present disclosure.

FIG. 2 is a block diagram illustrating an internal configuration of the robotic surgical system of FIG. 1.

FIG. 3 is a perspective view illustrating a slave robot of the robotic surgical system of FIG. 1 and surgical instruments mounted thereon.

FIG. 4 is a configuration diagram for describing an example of an internal configuration of a device for generating coordinate system transformation information, according to an embodiment.

FIG. 5 is a perspective view illustrating a surgical robot of the robotic surgical system of FIG. 1, and a surgical instrument mounted thereon.

FIG. 6 is a diagram illustrating a state in which an instrument case in FIG. 5 has been removed.

FIG. 7 is a perspective view illustrating a camera robot of the robotic surgical system of FIG. 1, and a laparoscopic surgical camera mounted thereon.

FIG. 8 is a diagram illustrating a state in which the surgical instrument is removed from the surgical robot of FIG. 5.

FIG. 9 is a perspective view illustrating another example of a surgical robot of a robotic surgical system, and a surgical instrument mounted thereon, according to an embodiment.

FIG. 10 is a perspective view illustrating an example of an angle measurement dial mounted on a robot, according to an embodiment.

FIG. 11 is a perspective view illustrating a surgical instrument according to an embodiment of the present disclosure.

FIGS. 12 and 13 are perspective views of an end tool of the surgical instrument of FIG. 11.

FIGS. 14A and 14B are plan views of an end tool of the surgical instrument of FIG. 11.

FIGS. 15 and 16 are perspective views of a driving part of the surgical instrument of FIG. 11.

FIG. 17 is a plan view of a driving part of the surgical instrument of FIG. 11.

FIG. 18 is a rear view of the driving part of the surgical instrument of FIG. 11.

FIG. 19 is a side view of the driving part of the surgical instrument of FIG. 11.

FIG. 20 is a diagram illustrating in detail components associated with a first jaw, among components of pulleys and wires of the surgical instrument illustrated in FIG. 11.

FIG. 21 is a diagram illustrating in detail components associated with a second jaw, among components of pulleys and wires of the surgical instrument illustrated in FIG. 11.

FIGS. 22A, 22B, and 22C and FIGS. 23A, 23B, and 23C are diagrams illustrating a pitch motion of the surgical instrument illustrated in FIG. 11.

FIGS. 24A and 24B and FIGS. 25A and 25B are diagrams illustrating a yaw motion of the surgical instrument illustrated in FIG. 11.

FIG. 26 is a flowchart for describing an example of a method of generating coordinate system transformation information in a robotic surgical system, according to an embodiment.

FIGS. 27 and 28 are perspective views for describing an example of a coordinate system used in a robotic surgical system, according to an embodiment.

FIG. 29 is a flowchart for describing another example of a method of generating coordinate system transformation information in a robotic surgical system, according to an embodiment.

FIGS. 30 and 31 are perspective views for describing another example of a coordinate system used in a robotic surgical system, according to an embodiment.

FIG. 32 is a diagram for describing an example of a robotic surgical system configured to operate based on coordinate system transformation information, according to an embodiment.

### MODE FOR INVENTION

Advantages and features of the present disclosure and a method for achieving them will be apparent with reference to embodiments of the present disclosure described below together with the accompanying drawings. The present disclosure may, however, be embodied in many different forms and should not be construed as being limited to the embodiments set forth herein, and all changes, equivalents, and substitutes that do not depart from the spirit and technical scope of the present disclosure are encompassed in the present disclosure. These embodiments are provided such that the present disclosure will be thorough and complete, and will fully convey the concept of the present disclosure to those of skill in the art. In describing the present disclosure, detailed explanations of the related art are omitted when it is deemed that they may unnecessarily obscure the gist of the present disclosure.

Terms used herein are for describing particular embodiments and are not intended to limit the scope of the present disclosure. Singular forms are intended to include plural forms as well, unless the context clearly indicates otherwise. In the present specification, it is to be understood that the terms such as “including,” “having,” and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Some embodiments of the present disclosure may be represented by functional block components and various processing operations. Some or all of the functional blocks may be implemented by any number of hardware and/or software elements that perform particular functions. For example, the functional blocks of the present disclosure may be embodied by at least one microprocessor or by circuit components for a certain function. In addition, for example, the functional blocks of the present disclosure may be implemented by using various programming or scripting languages. The functional blocks may be implemented by using various algorithms executable by one or more processors. Furthermore, the present disclosure may employ known technologies for electronic settings, signal processing, and/or data processing. Terms such as “mechanism,” “element,” “unit,” or “component” are used in a broad sense and are not limited to mechanical or physical components.

In addition, connection lines or connection members between components illustrated in the drawings are merely exemplary of functional connections and/or physical or circuit connections. Various alternative or additional functional connections, physical connections, or circuit connections between components may be present in a practical device.

Hereinafter, embodiments will be described in detail with reference to the accompanying drawings. The embodiments may, however, be embodied in many different forms and should not be construed as being limited to the embodiments set forth herein.

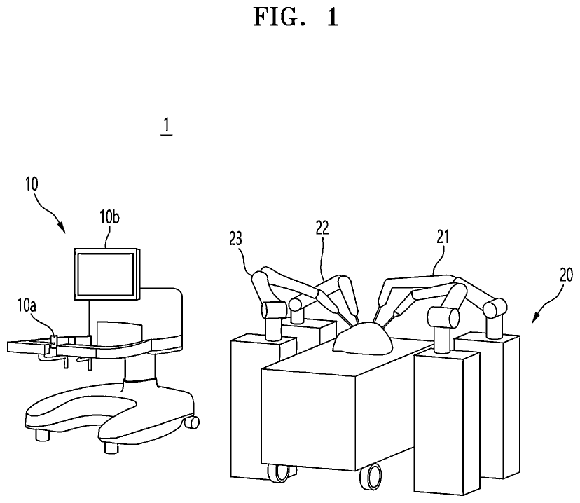

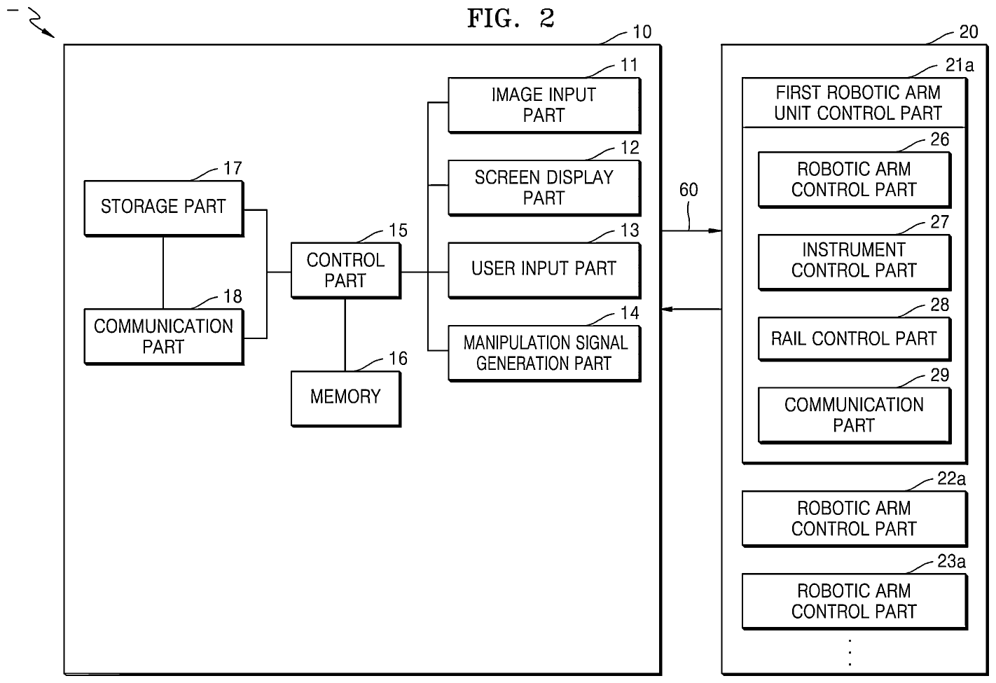

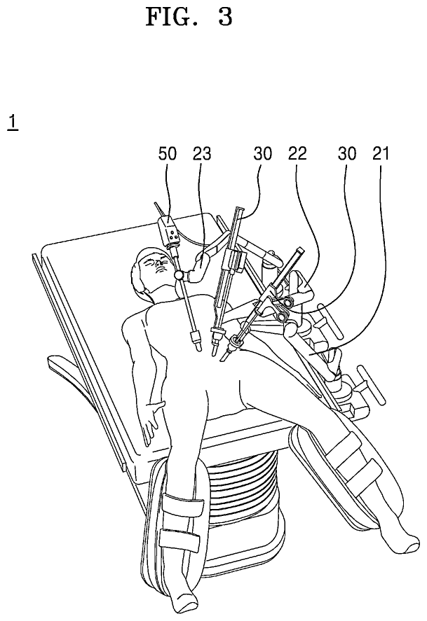

FIG. 1 is a conceptual diagram illustrating a robotic surgical system according to an embodiment of the present disclosure, FIG. 2 is a block diagram illustrating an internal configuration of the robotic surgical system of FIG. 1, and FIG. 3 is a perspective view illustrating a slave robot of the robotic surgical system of FIG. 1 and surgical instruments mounted thereon.

Referring to FIGS. 1 to 3, a surgical robotic system 1 includes a master robot 10, a slave robot 20, a surgical instrument 30, and a laparoscopic surgical camera 50.

The master robot 10 includes a manipulation member 10a and a display member 10b, and the slave robot 20 includes one or more robotic arm units 21, 22, and 23.

In detail, the master robot 10 includes the manipulation member 10a to allow an operator to hold and manipulate the manipulation member 10a with both hands. The manipulation member 10a may be implemented with two or more handles as illustrated in FIG. 1, and a manipulation signal according to manipulation of the handles by the operator is transmitted to the slave robot 20 through a wired or wireless communication network to control the robotic arm units 21, 22, and 23. That is, surgical motions such as positional movement, rotation, and cutting operations of the robotic arm units 21, 22, and 23 may be performed by manipulation of the handles by the operator.

For example, the operator may manipulate the robotic arm units 21, 22, and 23 by using handle-type manipulation levers. The manipulation lever may have various mechanical components depending on its manipulation method, and may be provided in various configurations for operating the robotic arm units 21, 22, and 23 of the slave robot 20 and/or other surgical instruments, such as a master handle for manipulating the motion of the robotic arm units 21, 22, and 23 and various input tools added to the master robot 10 for manipulating the functions of the entire system such as a joystick, a keypad, a trackball, a foot pedal, or a touch screen. Here, the manipulation member 10a is not limited to the shape of a handle and may be applied without any limitation as long as it may control motions of the robotic arm units 21, 22, and 23 through a network such as a wired or wireless communication network.

Alternatively, a voice input, a motion input, or the like may be applied to the surgical robotic system 1 for user input. That is, a user may wear, on his/her head, glasses or a head-mounted display (HMD) having a sensor attached thereto, and a laparoscopic surgical camera 50 may move according to the direction of the user's gaze. Alternatively, when the user issues a command with a voice, such as “left,” “right,” “first arm,” or “second arm,” the command may be recognized and the motion may be performed.

An image captured through the laparoscopic surgical camera 50 is displayed as a screen image on the display member 10b of the master robot 10. For example, the image captured through the laparoscopic surgical camera 50 may include a patient's surgical site, a surgical instrument inserted into the patient's surgical site, and a motion of the surgical instrument. For example, the display member 10b may display an image corresponding to a motion of the surgical instrument inserted into the patient's surgical site. In addition, a predetermined virtual manipulation panel may be displayed on the display member 10b independently of or together with the image captured through the laparoscopic surgical camera 50. Detailed descriptions of the arrangement and configuration of the virtual manipulation panel will be omitted.

Here, the display member 10b may include one or more monitors, and information necessary for surgery may be individually displayed on each monitor. The number of monitors may be determined in various ways depending on the class or type of information required to be displayed.

Meanwhile, one or more slave robots 20 may be provided to perform surgery on a patient. For example, the surgical robotic system 1 may include the slave robot 20 coupled to the surgical instrument 30, and the slave robot 20 coupled to the laparoscopic surgical camera 50. As such, the laparoscopic surgical camera 50 for displaying a surgical site as a screen image through the display member 10b may be implemented as an independent slave robot 20. In addition, as described above, embodiments of the present disclosure may be used universally for surgeries using various surgical endoscopes other than laparoscopes (e.g., thoracoscopes, arthroscopes, or rhinoscopes).

Meanwhile, for example, surgical instruments 30 may be attached to two of the robotic arm units 21, 22, and 23, and the laparoscopic surgical camera 50 may be attached to the other one. In addition, a surgeon, i.e., an operator, may select the slave robot 20 (or the robotic arm unit 21, 22, or 23) to be controlled through the master robot 10. As such, the surgeon may directly control a total of three or more surgical instruments through the master robot 10 without the need for a surgical assistant, and thus manipulate various instruments accurately and freely as intended by the surgeon.

As another example, the slave robot 20 may include one or more robotic arm units 21, 22, and 23. For example, although FIGS. 1 to 3 illustrate that one robotic arm unit 21, 22, or 23 is coupled to one slave robot 20, the present disclosure is not limited thereto. For example, two robotic arm units may be combined with one slave robot 20 such that the surgical instrument 30 is attached to any one of the robotic arm units, and the laparoscopic surgical camera 50 is attached to the other robotic arm unit. However, even in a case in which a plurality of robotic arm units are combined with one slave robot 20, the robotic arm units 21, 22, and 23 may be provided as respective modules capable of operating independently of each other, and in this case, an algorithm for preventing collisions between the robotic arm unit 21, 22, and 23 may be applied to the surgical robotic system 1.

In general, a robotic arm has functions similar to a human arm and/or wrist, and refers to a device to which a certain tool may be attached to a wrist area thereof. In the present disclosure, the robotic arm unit 21, 22, or 23 may be defined as a concept that encompasses all components such as an upper arm, a lower arm, a wrist, or an elbow, and a surgical instrument (or a laparoscopic surgical camera) coupled to the wrist area. Alternatively, it may be defined as a concept that encompasses only components for driving the surgical instrument (or a laparoscopic surgical camera), excluding the surgical instrument (or the laparoscopic surgical camera) coupled to the wrist area.

As such, the robotic arm units 21, 22, and 23 of the slave robot 20 may be implemented to be driven with multiple degrees of freedom. The robotic arm units 21, 22, and 23 may include, for example a surgical instrument (or a laparoscopic surgical camera) inserted into a surgical site of a patient, a yaw driving part for rotating the surgical instrument in a yaw direction according to a surgical position, a pitch driving part for rotating the surgical instrument in a pitch direction orthogonal to the rotation drive of the yaw driving part, a transfer driving part for moving the surgical instrument in the longitudinal direction, a rotation driving part for rotating the surgical instrument, and a surgical instrument driving part for driving an end effector at an end of the surgical instrument to incise or cut a surgical lesion. However, the configurations of the robotic arm units 21, 22, and 23 are not limited thereto, and it should be understood that this example does not limit the scope of the present disclosure. Here, a detailed description of an actual control process, such as rotation or movement of the robotic arm unit 21, 22, and 23 in a corresponding direction when the operator manipulates the manipulation member 10a, will be omitted.

Referring to FIG. 2, in an embodiment of the present disclosure, the master robot 10 may include an image input part 11, a screen display part 12, a user input part 13, a manipulation signal generation part 14, a control part 15, a memory 16, a storage part 17, and a communication part 18.

The image input part 11 may receive, through a wired or wireless communication network, an image captured through a camera provided in the laparoscopic surgical camera 50 of the slave robot 20. For example, an image captured through the camera provided in the laparoscopic surgical camera 50 may include the patient's surgical site, a surgical instrument inserted into the patient's surgical site, and a motion of the surgical instrument.

The screen display part 12 outputs, as visual information, a screen image corresponding to the image received through the image input part 11. In addition, when biometric information of a patient is input, the screen display part 12 may further output information corresponding to the biometric information. In addition, the screen display part 12 may further output image data related to the patient's surgical site (e.g., an X-ray image, a computed tomography (CT) image, or a magnetic resonance imaging (MRI) image). Here, the screen display part 12 may be implemented in the form of a display member (see 10b of FIG. 1), and an image process for outputting the received image as a screen image through the screen display part 12 may be performed by the control part 15.

In the embodiment illustrated in FIG. 2, the image input part 11 and the screen display part 12 are illustrated as components included in the master robot 10, but are not limited thereto. The display member 10b may be provided as a separate member spaced apart from the master robot 10. Alternatively, the display member 10b may be provided as a component of the master robot 10. In addition, in another embodiment, a plurality of display members 10b may be provided, one of which may be arranged adjacent to the master robot 10, and some of which may be arranged at a position that is spaced a little apart from the master robot 10.

Here, the screen display part 12 (i.e., the display member 10b of FIG. 1) may be provided as a stereoscopic display device. In detail, the stereoscopic display device refers to an image display device for applying stereoscopic technology to add depth information to a two-dimensional image, and enabling a viewer to feel the liveliness and reality of three dimensions by using the depth information. The surgical robotic system 1 according to an embodiment of the present disclosure may include a stereoscopic display device as the screen display part 12 to provide a more realistic virtual environment to a user.

The user input part 13 is a unit for allowing the operator to control the positions and functions of the robotic arm units 21, 22, and 23 of the slave robot 20. The user input part 13 may be formed in the form of a handle-shaped manipulation member (see 10a of FIG. 1) as illustrated in FIG. 1, but its shape is not limited thereto, and the user input part 13 may be implemented in various shapes to achieve the same purpose. In addition, for example, some user input units may be formed in the shape of a handle, and others may be formed in the shape of a clutch button, and a finger insertion tube or insertion tube ring may be further formed so as to allow the operator's finger to be inserted and fixed to facilitate manipulation of the surgical tool.

When the operator manipulates the user input part 13 to move the position of the robotic arm unit 21, 22, or 23 or manipulate a surgical motion thereof, the manipulation signal generation part 14 may generate a manipulation signal corresponding to the manipulation. For example, when the operator manipulates the user input part 13 to move the position of the robotic arm unit 21, 22, or 23 or manipulate a surgical motion thereof, the manipulation signal generation part 14 may generate manipulation information corresponding to the manipulation.

For example, the manipulation signal generation part 14 transmits the generated manipulation signal to the control part 15, or to the slave robot 20 through the communication part 18. The manipulation signal may be transmitted and received through a wired or wireless communication network. Based on the transmitted manipulation signal, the control part 15 may control the slave robot 20, the surgical instrument 30, or the laparoscopic surgical camera 50 to operate. Alternatively, based on the transmitted manipulation signal, a robotic arm control part 26 included in the slave robot 20 may control the robotic arm units 21, 22, and 23 to operate. Alternatively, based on the transmitted manipulation signal, an instrument control part 27 included in the slave robot 20 may control the surgical instrument 30 or the laparoscopic surgical camera 50 to operate. However, a method of controlling the operation of the slave robot 20, the surgical instrument 30, or the laparoscopic surgical camera 50 based on a manipulation signal is not limited to the above.

The control part 15 is a kind of central processing unit and controls the operation of each component such that the above-described functions may be performed. For example, the control part 15 may perform a function of converting an image input through the image input part 11 into a screen image to be displayed through the screen display part 12.

The memory 16 may perform a function of temporarily or permanently storing data processed by the control part 15. Here, the memory 16 may include a magnetic storage medium or a flash storage medium, but the scope of the present disclosure is not limited thereto.

The storage part 17 may store data received from the slave robot 20. In addition, the storage part 17 may store various types of input data (e.g., patient data, device data, or surgery data).

The communication part 18 provides a communication interface necessary to transmit and receive image data transmitted from the slave robot 20 and control data transmitted from the master robot 10, in conjunction with a communication network 60.

The slave robot 20 includes a plurality of robotic arm unit control parts 21a, 22a, and 23a. In addition, the robotic arm unit control part 21a includes the robotic arm control part 26, the instrument control part 27, and a communication part 29. In addition, the robotic arm unit control part 21a may further include a rail control part 28.

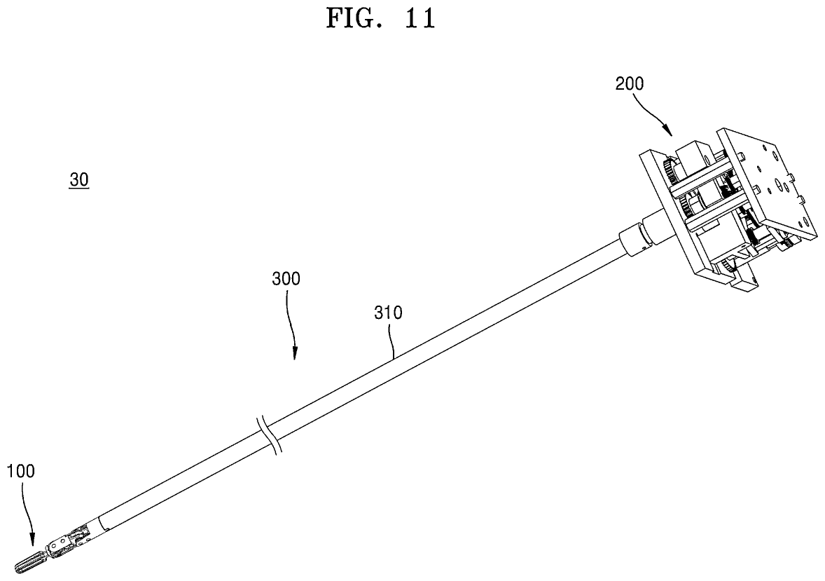

Referring to FIGS. 2 and 3, the rail control part 28 may control a movement path such that the surgical instrument 30 in the robotic arm unit 21, 22, or 23 may move along a preset path, in detail, in the longitudinal direction of a connection part 310, which will be described below with reference to FIG. 11.

The robotic arm control part 26 may serve to receive a manipulation signal generated by the manipulation signal generation part 14 of the master robot 10 and control the robotic arm unit 21, 22, and 23 to operate according to the manipulation signal.

The instrument control part 27 may serve to receive a manipulation signal generated by the manipulation signal generation part 14 of the master robot 10 and control the surgical instrument 30 to operate according to the manipulation signal.

The communication part 29 provides a communication interface necessary to transmit and receive image data transmitted from the slave robot 20 and control data transmitted from the master robot 10, in conjunction with the communication network 60.

Meanwhile, the communication network 60 serves to connect the master robot 10 to the slave robot 20. That is, the communication network 60 refers to a communication network for providing a connection path such that the master robot 10 and the slave robot 20 may transmit and receive data to and from each other after being connected to each other. The network 60 may include, for example, a wired network such as a local area network (LAN), a wide area network (WAN), a metropolitan area network (MAN), or an integrated services digital network (ISDN), or a wireless network such as a wireless LAN (WLAN), code-division multiple access (CDMA), Bluetooth, or satellite communication, but the scope of the present disclosure is not limited thereto.

Meanwhile, the operator manipulating the master robot 10 may generally operate the manipulation member 10a while viewing a screen image displayed on the display member 10b. For example, the operator may manipulate a motion of the surgical instrument according to the intention of the operator through the manipulation member 10a, while checking the motion of the surgical instrument included in the screen image in real time.

Here, manipulating the motion of the surgical instrument according to the intention of the operator may include a process of converting movement information of the manipulation member 10a of the master robot 10 manipulated by the operator, into movement information of the surgical instrument.

In other words, in order for the operator to intuitively control the movement of the surgical instrument, a process for conversion between movement information of the surgical instrument on the screen image, movement information of the manipulation member 10a manipulated by the operator, and actual movement information of the surgical instrument, may be necessary.

Meanwhile, as described above, because the operator manipulates the manipulation member 10a while viewing the screen image displayed on the display member 10b, it may be assumed that the operator uses a coordinate system used for the screen image. That, it may be assumed that the movement information of the surgical instrument on the screen image is the same as the movement information of the manipulation member 10a manipulated by the operator.

In contrast, a method of calculating the conversion between the movement information of the surgical instrument on the screen image and the actual movement information of the surgical instrument may depend on a method of configuring the slave robot 20 or the robotic arm unit 21, 22, or 23.

For example, in a case in which both the surgical instrument 30 and the laparoscopic surgical camera 50 are coupled to the robotic arm unit 21, the surgical instrument 30 and the laparoscopic surgical camera 50 use the same coordinate system. In this case, because the movement information of the surgical instrument 30 on the screen image and the actual movement information of the surgical instrument 30 are the same, there is no need to calculate conversion between the movement information thereof.

As another example, in a case in which the surgical instrument 30 is coupled to the robotic arm unit 22 and the laparoscopic surgical camera 50 is coupled to the robotic arm unit 23, in other words, in a case in which the surgical instrument 30 and the laparoscopic surgical camera 50 are mounted on separate robotic arm units, the surgical instrument 30 and the laparoscopic surgical camera 50 use different coordinate systems. That is, because the surgical instrument 30 and the laparoscopic surgical camera 50 are coupled to independent robotic arm units, the movement information of the surgical instrument 30 in the screen image may be movement information determined through a certain conversion process on the actual movement information of the surgical instrument 30. In addition, the actual movement information of the surgical instrument 30 may be movement information determined through a certain conversion process on the movement information of the surgical instrument 30 in the screen image. That is, in order to calculate conversion between the movement information of the surgical instrument 30 in the screen image and the actual movement information of the surgical instrument 30, it is necessary to calculate coordinate system transformation information between the coordinate system used by the laparoscopic surgical camera 50 and the coordinate system used by the surgical instrument 30.

As another example, the movement information of the surgical instrument 30 coupled to the robotic arm unit 21 may be determined in relation to the movement information of the surgical instrument 30 coupled to the robotic arm unit 22. For example, the surgical instrument 30 coupled to the robotic arm unit 21 may need to move parallel or perpendicular to the surgical instrument 30 coupled to the robotic arm unit 22, for a certain motion. In this case, movements between a plurality of surgical instruments may be easily controlled by calculating coordinate system transformation information between the coordinate system used by the surgical instrument 30 coupled to the robotic arm unit 21, and the coordinate system used by the surgical instrument 30 coupled to the robotic arm unit 22.

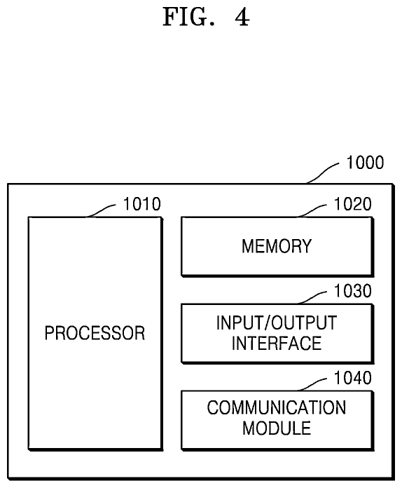

Hereinafter, a device for generating coordinate system transformation information between a robot included in the robotic surgical system according to an embodiment of the present disclosure (e.g., a robot equipped with the surgical instrument 30 (hereinafter, referred to as a ‘surgical robot’) and robots equipped with laparoscopic surgical cameras 50 (hereinafter, referred to as a ‘camera robots’) will be described with reference to FIG. 4.

FIG. 4 is a configuration diagram for describing an example of an internal configuration of a device for generating coordinate system transformation information, according to an embodiment.

Referring to FIG. 4, a device 1000 for generating coordinate system transformation information (hereinafter, referred to as a “device”) includes a processor 1010, a memory 1020, an input/output interface 1030, and a communication module 1040. For convenience of description, FIG. 4 illustrates only components related to the present disclosure. Thus, other general-purpose components than those illustrated in FIG. 4 may be further included in the device 1000. In addition, it is obvious to those of skill in the art related to the present disclosure that the processor 1010, the memory 1020, the input/output interface 1030, and the communication module 1040 illustrated in FIG. 4 may also be implemented as independent devices.

The processor 1010 may process commands of a computer program by performing basic arithmetic, logic, and input/output operations. Here, the commands may be provided from the memory 1020 or an external device (e.g., an external server (not shown)). In addition, the processor 1010 may control the overall operation of other components included in the device 1000.

The processor may calculate an angle difference between a preset reference coordinate system and a robot base coordinate system, based on a dial manipulation value of a user obtained from an angle measurement dial mounted on a robot. For example, the processor may calculate an angle difference between the preset reference coordinate system and a camera robot base coordinate system, based on a dial manipulation value of the user obtained from an angle measurement dial mounted on a camera robot. As another example, the processor may calculate an angle difference between the preset reference coordinate system and a surgical robot base coordinate system, based on a dial manipulation value of the user obtained from an angle measurement dial mounted on a surgical robot. That is, an angle difference (e.g., the angle difference between the reference coordinate system and the camera robot base coordinate system or the angle difference between the reference coordinate system and the surgical robot base coordinate system) may be calculated based on a dial manipulation value of the user obtained from an angle measurement dial.

In addition, an angle measurement dial (e.g., the angle measurement dial mounted on the camera robot or the angle measurement dial mounted on the surgical robot) may receive a physical dial manipulation by the user to obtain a dial manipulation value corresponding to an angle difference.

According to an embodiment, in order to generate coordinate system transformation information between a first robot and a second robot both included in the robotic surgical system, the processor may calculate a first angle difference between the reference coordinate system and a first robot base coordinate system, and a second angle difference between the reference coordinate system and a second robot base coordinate system. Here, the first angle difference may be calculated based on a dial manipulation value of the user obtained from a first angle measurement dial mounted on the first robot, and the second angle difference may be calculated based on a dial manipulation value of the user obtained from a second angle measurement dial mounted on the second robot. Meanwhile, each of the first robot and the second robot both included in the robotic surgical system may be a camera robot or a surgical robot.

According to an embodiment, in a case in which the first angle measurement dial (refers to, but not limited to, an angle measurement dial mounted on the first robot) does not use the first robot base coordinate system, the processor may calculate a third angle difference between the reference coordinate system and a coordinate system used by the first angle measurement dial, based on a dial manipulation value obtained from the first angle measurement dial.

Here, the first angle measurement dial may be mounted at, among positions on the first robot at which the first angle measurement dial may be mounted, a position on the first robot other than a position serving as a reference point of the first robot base coordinate system.

In addition, the processor may calculate a fifth angle difference between a first angle measurement dial coordinate system and the first robot base coordinate system, based on a first angle measurement value obtained from a first angle measurement sensor mounted on a robotic arm unit of the first robot.

According to an embodiment, in a case in which the second angle measurement dial (refers to, but not limited to, an angle measurement dial mounted on the second robot) does not use the second robot base coordinate system, the processor may calculate a fourth angle difference between the reference coordinate system and a coordinate system used by the second angle measurement dial, based on a dial manipulation value obtained from the second angle measurement dial.

Here, the second angle measurement dial may be mounted at, among positions on the first robot at which the second angle measurement dial may be mounted, a position on the second robot other than a position serving as a reference point of the second robot base coordinate system.

In addition, the processor may calculate a sixth angle difference between a second angle measurement dial coordinate system and the second robot base coordinate system, based on a second angle measurement value obtained from a second angle measurement sensor mounted on a robotic arm unit of the second robot.

Meanwhile, the processor may generate coordinate system transformation information between the first robot base coordinate system and the second robot base coordinate system, based on the first angle difference and the second angle difference.

For example, the processor may generate first coordinate system transformation information between the first robot base coordinate system and the reference coordinate system by using the first angle difference, generate second coordinate system transformation information between the second robot base coordinate system and the reference coordinate system by using the second angle difference, and generate coordinate system transformation information between the first robot base coordinate system and the second robot base coordinate system based on the first coordinate system transformation information and the second coordinate system transformation information.

Meanwhile, according to an embodiment, the preset reference coordinate system may include any one of a coordinate system used by a surgical bed in an operating room where the first robot and the second robot are arranged, or a coordinate system used by a master robot included in the robotic surgical system.

According to an embodiment, the processor may calculate a relative angle between the first robot base coordinate system and the second robot base coordinate system by using the first angle difference and the second angle difference, and calculate a rotation transformation matrix between the first robot base coordinate system and the second robot base coordinate system based on the relative angle.

According to another embodiment, the processor may calculate a rotation transformation matrix between the reference coordinate system and the first angle measurement dial coordinate system by using the third angle difference, and calculate a rotation transformation matrix between the first angle measurement dial coordinate system and the first robot base coordinate system by using the fifth angle difference, so as to generate first coordinate system transformation information between the first robot base coordinate system and the reference coordinate system. In addition, the processor may calculate a rotation transformation matrix between the reference coordinate system and the second angle measurement dial coordinate system by using the fourth angle difference, and calculate a rotation transformation matrix between the second angle measurement dial coordinate system and the second robot base coordinate system by using the sixth angle difference, so as to generate second coordinate system transformation information between the second robot base coordinate system and the reference coordinate system.

Meanwhile, according to an embodiment, the first angle difference or the second angle difference may refer to, among roll values, pitch values, and yaw values, a difference in yaw value of the reference point of the first robot base coordinate system with respect to the reference coordinate system, or a difference in yaw value of the reference point of the second robot base coordinate system with respect to the reference coordinate system. In addition, the third angle difference or the fourth angle difference may refer to, among roll values, pitch values, and yaw values, a difference in yaw value of the reference point of the first angle measurement dial coordinate system with respect to the reference coordinate system, or a difference in yaw value of the reference point of the second angle measurement dial coordinate system with respect to the reference coordinate system. In addition, the fifth angle difference may refer to a difference in yaw value of the reference point of the first robot base coordinate system with respect to the first angle measurement dial coordinate system, and the sixth angle difference may refer to a difference in yaw value of the reference point of the second robot base coordinate system with respect to the second angle measurement dial coordinate system.

Meanwhile, according to an embodiment, the processor may initialize the generated coordinate system transformation information after surgery is terminated. That is, the processor may initialize previously generated coordinate system transformation information in response to termination of surgery performed based on the generated coordinate system transformation information. This may be for generating new coordinate system transformation information as the position or direction of a surgical robot or a camera robot may change when performing new surgery.

According to an embodiment, the processor may monitor coordinate system transformation information before surgery begins, and when coordinate system transformation information is not generated or when the monitored coordinate system transformation information corresponds to initialized coordinate system transformation information, generate an angle measurement dial manipulation alarm signal. For example, when new surgery is to be performed but a user (e.g., an operator) does not manipulate the angle measurement dial and thus coordinate system transformation information is not generated, or when coordinate system transformation information that has been initialized as the previous surgery is terminated remains as it is, the processor may generate an angle measurement dial manipulation alarm signal to notify the user that the angle measurement dial needs to be manipulated. For example, the processor may transmit the angle measurement dial manipulation alarm signal directly to the first angle measurement dial or the second angle measurement dial, and the first angle measurement dial or the second angle measurement dial may provide a notification to the user through a notification part (not shown). As another example, the processor may transmit the angle measurement dial manipulation alarm signal to the communication part 18 or the communication part 29, and in response to the communication part 18 or the communication part 29 receiving the signal, the control part 15 or the robotic arm control part 26 may control the master robot 10 or the slave robot 20 to provide the user with an alarm for notifying that the user needs to manipulate the angle measurement dial.

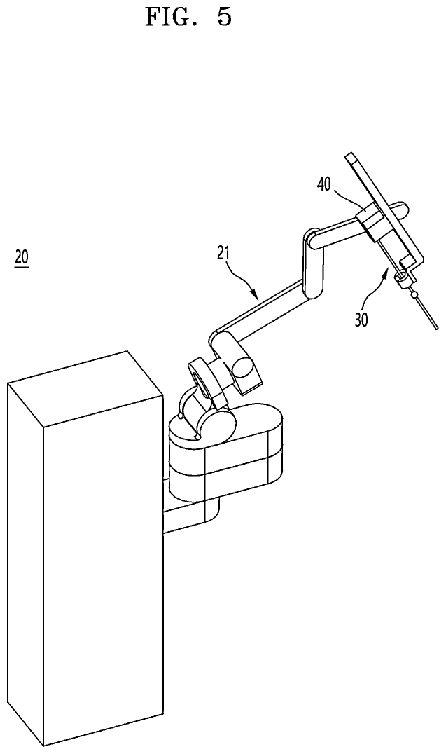

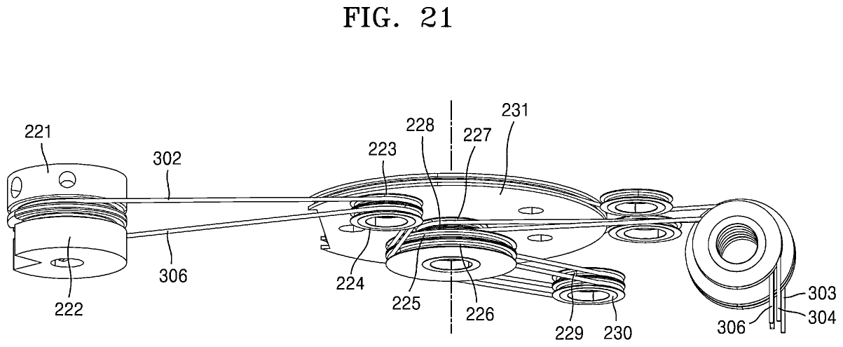

Detailed examples in which the processor 1010 according to an embodiment operates will be described with reference to FIGS. 5 to 32.

The processor 1010 may be implemented as an array of a plurality of logic gates, or may be implemented as a combination of a general-purpose microprocessor and a memory storing a program executable by the microprocessor. For example, the processor 1010 may include a general-purpose processor, a central processing unit (CPU), a microprocessor, a digital signal processor (DSP), a controller, a microcontroller, a state machine, and the like. In some environments, the processor 1010 may include an application-specific integrated circuit (ASIC), a programmable logic device (PLD), a field-programmable gate array (FPGA), and the like. For example, processor 1010 may refer to a combination of processing devices, such as a combination of a DSP and a microprocessor, a combination of a plurality of microprocessors, a combination of one or more microprocessors combined with a DSP core, or a combination of any other such configurations.

The memory 1020 may include any non-transitory computer-readable recording medium. For example, the memory 1020 may include a permanent mass storage device, such as random-access memory (RAM), read-only memory (ROM), a disk drive, a solid-state drive (SSD), or flash memory. As another example, the permanent mass storage device, such as ROM, an SSD, flash memory, or a disk drive, may be a permanent storage device separate from the memory. Also, the memory 1020 may store an operating system (OS) and at least one piece of program code (e.g., code for the processor 1010 to perform an operation to be described below with reference to FIGS. 5 to 32).

According to an embodiment, the memory 1020 may store coordinate system transformation information between the first robot base coordinate system and the second robot base coordinate system.

These software components may be loaded from a computer-readable recording medium separate from the memory 1020. The separate computer-readable recording medium may be a recording medium that may be directly connected to the device 1000, and may include, for example, a computer-readable recording medium, such as a floppy drive, a disk, a tape, a digital video disc (DVD)/compact disc ROM (CD-ROM) drive, or a memory card.

Alternatively, the software components may be loaded into the memory 1020 through the communication module 1040 rather than a computer-readable recording medium. For example, at least one program may be loaded into the memory 1020 on the basis of a computer program (e.g., a computer program for the processor 1010 to perform an operation to be described below with reference to FIGS. 5 to 32) installed by files provided via the communication module 1040 by developers or a file distribution system that distributes installation files of applications.

The input/output interface 1030 may be a unit for an interface with a device (e.g., a keyboard or a mouse) for input or output that may be connected to the device 1000 or included in the device 1000. The input/output interface 1030 may be implemented separately from the processor 1010, but is not limited thereto, and may be implemented to be included in the processor 1010.

The communication module 1040 may provide a configuration or function for the device 1000 to communicate with an external device (e.g., the communication part 18 or the communication part 29) through a network. In addition, the communication module 1040 may provide a configuration or function for the device 1000 to communicate with other external devices. For example, a control signal, a command, data, and the like provided under control of the processor 1010 may be transmitted to an external device through the communication module 1040 and a network.

According to an embodiment, the communication module 1040 may transmit the coordinate system transformation information between the first robot base coordinate system and the second robot base coordinate system, to an external device (e.g., the communication part 18 or the communication part 29).

Meanwhile, according to an embodiment, the device 1000 may be a component included in the master robot 10. For example, the master robot 10 may further include a coordinate system transformation information generation part (not shown), and the operations performed by the device 1000 may be performed by the coordinate system transformation information generation part (not shown) included in the master robot 10. As another example, the operations performed by the processor 1010, memory 1020, the input/output interface 1030, and the communication module 1040 may be performed by the control part 15, the memory 16, the screen display part 12, the user input part 13, and the communication part 18, which are included in the master robot 10.

According to another embodiment, the device 1000 may be a component included in a robotic arm unit control part of a surgical robot, or in a robotic arm unit control part of a camera robot. For example, the robotic arm unit control part of the surgical robot or the robotic arm unit control part of the camera robot may further include a coordinate system transformation information generation part (not shown), and the operations performed by the device 1000 may be performed by the coordinate system transformation information generation part (not shown) included in the robotic arm unit control unit of the surgical robot or the robotic arm unit control unit of the camera robot.

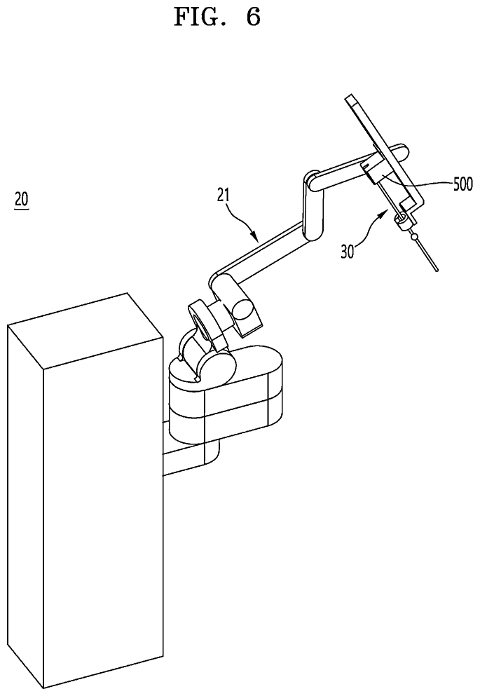

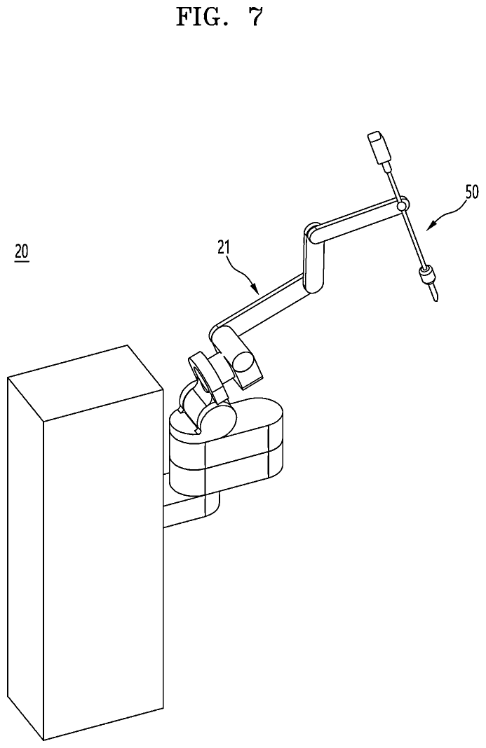

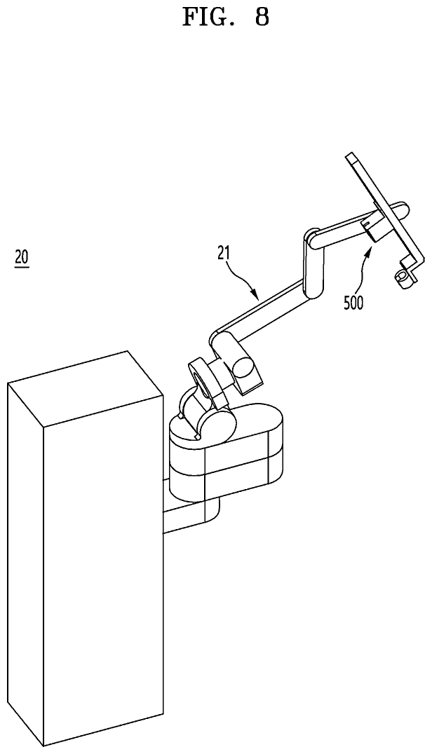

FIG. 5 is a perspective view illustrating a surgical robot of the robotic surgical system of FIG. 1, and a surgical instrument mounted thereon. FIG. 6 is a diagram illustrating a state in which an instrument case in FIG. 5 has been removed. FIG. 7 is a perspective view illustrating a camera robot of the robotic surgical system of FIG. 1, and a laparoscopic surgical camera mounted thereon. FIG. 8 is a diagram illustrating a state in which the surgical instrument is removed from the surgical robot of FIG. 5.

The surgical instrument 30 or the laparoscopic surgical camera 50, which will be described below, may be connected to and installed in the robotic arm unit 21, 22, or 23. Referring to FIG. 5, an instrument case 40 may cover the surgical instrument 30, and may be connected to the robotic arm unit 21. The instrument case 40 may cover one side of the surgical instrument 30 exposed to the outside, so as to prevent external foreign substances from reaching the surgical instrument 30, and protect the surgical instrument 30 from being damaged due to external shock.

Referring to FIG. 6, the surgical instrument 30 may be connected to and installed in the robotic arm unit 21 of the surgical robot according to an embodiment. Referring to FIG. 7, the laparoscopic surgical camera 50 may be connected to and installed in the robotic arm unit 21 of the camera robot according to an embodiment.

Meanwhile, FIGS. 5 to 8 illustrate only one of the robotic arm units 21, 22, and 23, which is combined with the surgical instrument 30 or the laparoscopic surgical camera 50, but the present disclosure is not limited thereto, and as described above, the surgical instruments 30 may be attached to two of the robotic arm units 21, 22, and 23, and the laparoscopic surgical camera 50 may be attached to the other one.

Referring to FIGS. 5 to 8, a motor pack 500 is connectable to the surgical instrument 30, and may be coupled to the slave robot 20, specifically, the robotic arm unit 21, and fixed in position.

The instrument case 40 may be connected to one side of the surgical instrument 30, and the motor pack 500 may be connected to the opposite side. The motor pack 500 is for generating power by receiving power from the outside, and may transmit the power generated by the motor pack 500 to the surgical instrument 30, and accordingly, the surgical instrument 30 may perform a pitch motion, a yaw motion, an actuation motion, and a roll motion.

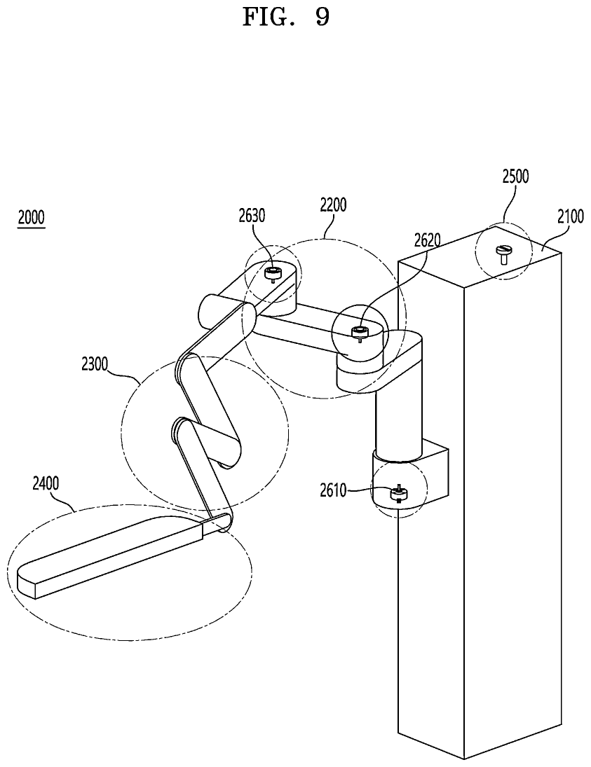

FIG. 9 is a perspective view illustrating another example of a surgical robot of a robotic surgical system, and a surgical instrument mounted thereon, according to an embodiment.

Referring to FIG. 9, a surgical robot 2000 according to an embodiment may include a body 2100, an active arm unit 2300, a surgical instrument 2400, and an angle measurement dial 2500. In addition, the surgical robot 2000 according to another embodiment may further include a passive arm unit 2200 and one or more angle measurement sensors 2610, 2620, and 2630.

The body 2100 may refer to a main body connected to a robotic arm unit. For example, the robotic arm unit and body 2100 may constitute one independent slave robot 20. In addition, the body 2100 may include a moving means (not shown) for allowing the surgical robot 2000 to be arranged at a desired position in an operating room. For example, the body 2100 may be equipped with wheels to be freely movable. Meanwhile, the body 2100 may further include a fixing means (not shown) for fixing the surgical robot 2000 in the operating room to be prevented from moving. For example, after the surgical robot 2000 is completely arranged and an operator begins surgery, the fixing means may fix the body 2100 at a certain position in the operating room such that the surgical robot 2000 cannot move for the stability of the surgery.

The robotic arm unit included in the surgical robot 2000 may include at least one of the passive arm unit 2200 and the active arm unit 2300. For example, the surgical robot 2000 may be composed of the body 2100 and the active arm unit 2300, or may be composed of the body 2100, the passive arm unit 2200, and the active arm unit 2300. For example, in a case in which the robotic arm unit of the surgical robot 2000 is composed of only the active arm unit 2300, the active arm unit 2300 may be directly connected to the body 2100. As another example, in a case in which the robotic arm unit of the surgical robot 2000 is composed of the passive arm unit 2200 and the active arm unit 2300, the body 2100 may be directly connected to the passive arm unit 2200, one end of the passive arm unit 2200 may be connected to the body 2100, and the other end may be connected to the active arm unit 2300.

The passive arm unit 2200 may be defined as a robotic arm of which the position, direction, angle, and the like are manipulated by external forces. For example, the operator or a surgical assistant assisting the operator may manipulate the movement of the passive arm unit 2200 by applying a physical force. In addition, when there is no external force for manipulating the movement of the passive arm unit 2200, the passive arm unit 2200 may maintain its position, direction, angle, and the like. In other words, when the above-described operator or surgical assistant manipulates the position, direction, angle, and the like before the beginning of surgery, the position, direction, angle, and the like of the passive arm unit 2200 may remain unchanged while the surgery is in progress. In this respect, the body 2100 may be included in the passive arm unit 2200, because the position of the body 2100 that is moved by the operator or surgical assistant before the beginning of the surgery may remain unchanged while the surgery is in progress.

Meanwhile, the passive arm unit 2200 may include the angle measurement sensors 2610, 2620, and 2630. Here, the angle measurement sensors 2610, 2620, and 2630 may refer to sensors for monitoring the movement of the passive arm unit 2200. For example, the angle measurement sensors 2610, 2620, and 2630 may measure or calculate the position, direction, angle, and the like of the passive arm unit 2200. For example, the angle measurement sensors 2610, 2620, and 2630 may be implemented as sensors capable of measuring changes in the position, speed, and direction of a target, such as a rotary encoder, a linear encoder, or a potentiometer.

In addition, each of the angle measurement sensors 2610, 2620, and 2630 may be installed between any two passive arm units. For example, the number of angle measurement sensors included in the surgical robot 2000 may be one less than the number of passive arm units 2200. Referring to FIG. 9, the passive arm unit 2200 connecting the body 2100 to the active arm unit 2300 may include a total of four robotic arms, and the surgical robot 2000 according to an embodiment may include a total of three angle measurement sensors.

The active arm unit 2300 may be defined as a robotic arm of which the position, direction, angle, and the like are automatically manipulated through an internal control algorithm. For example, when the operator manipulates the user input part 13 to manipulate the active arm unit 2300, the manipulation signal generation part 14 may generate a manipulation signal corresponding to a motion of the operator manipulating the user input part 13, and transmit the manipulation signal to a robotic arm control part of the active arm unit 2300. Thereafter, the robotic arm control part of the active arm unit 2300 may control the active arm unit 2300 to perform a position movement, rotation, or the like according to a control algorithm, based on the received control signal. In other words, the position, direction, angle, and the like of the active arm unit 2300 may be manipulated when there is manipulation by the operator, regardless of whether before or after the beginning of surgery. Meanwhile, because the active arm unit 2300 is manipulated through the control algorithm rather than an external force, supply of external energy through a motor or an actuator is required. Thus, the active arm unit 2300 may include one or more motors or actuators.

The surgical instrument 2400 included in the surgical robot 2000 may be connected to at least one of the passive arm unit 2200 and the active arm unit 2300. Meanwhile, FIG. 9 illustrates the surgical robot 2000 to which the surgical instrument 2400 is coupled, but the present disclosure is not limited thereto. That is, the descriptions with reference to FIG. 9 may be equally applied to a camera robot to which a laparoscopic surgical camera (not shown) is coupled.

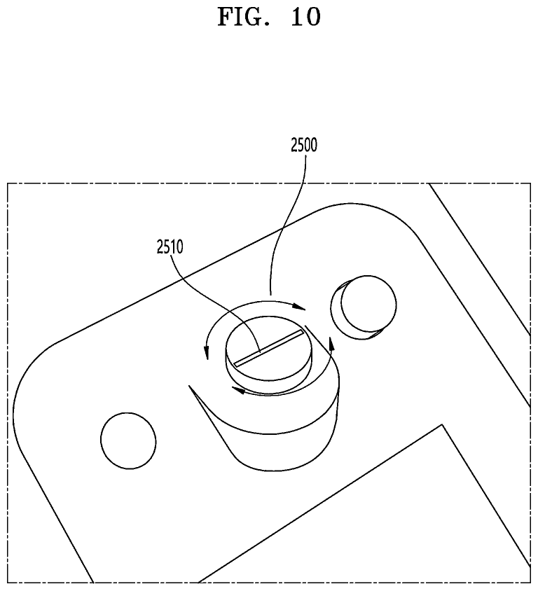

Meanwhile, the surgical robot 2000 or the camera robot may include the angle measurement dial 2500. Hereinafter, the angle measurement dial 2500 that may be included in the slave robot 20 (e.g., a surgical robot or a camera robot) according to an embodiment of the present disclosure will be described in detail with reference to FIG. 10.

FIG. 10 is a perspective view illustrating an example of an angle measurement dial mounted on a robot, according to an embodiment.

Referring to FIG. 10, the slave robot 20 according to an embodiment of the present disclosure may include the angle measurement dial 2500. That is, the angle measurement dial 2500 may be installed on each of the surgical robot and the camera robot.

The angle measurement dial 2500 may be defined as a rotary manipulation device for obtaining a dial manipulation value of a user by receiving physical manipulation by the user manipulating a dial. Here, the dial manipulation value of the user may correspond to an angle difference calculated by a coordinate system transformation information generation device by using a dial manipulation value. Meanwhile, the angle measurement dial 2500 may be implemented as a rotary dial such as a jog dial. The angle measurement dial 2500 is able to obtain a dial manipulation value of a user (e.g., a manipulation value for an angle by which the user rotates the dial) with only intuitive and simple manipulation by the user.

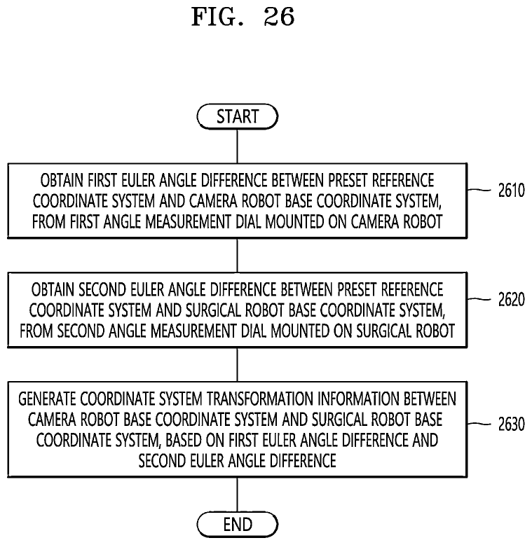

For example, the angle measurement dial 2500 may be implemented in a circular shape to receive dial manipulation by the user (e.g., rotation manipulation). For example, the user may measure an angle by rotating the angle measurement dial 2500 clockwise or counterclockwise. For example, the user may measure, through the angle measurement dial 2500, an azimuth of a slave robot equipped with the corresponding angle measurement dial, with respect to a reference coordinate system. This will be described in detail with reference to FIGS. 26 to 32.

Meanwhile, the shape, size, material, and the like of the angle measurement dial 2500 are not limited to the example illustrated in FIG. 10. The angle measurement dial 2500 according to an embodiment of the present disclosure may include a rotary dial that meets the definition of the angle measurement dial 2500 described above, in addition to the example illustrated in FIG. 10.

According to an embodiment, the angle measurement dial 2500 may include a marking 2510. For example, the angle measurement dial 2500 having the marking 2510 added thereto may provide the user with a more intuitive and simple angle measurement method.

According to an embodiment, the angle measurement dial 2500 may be manipulated based on rotation manipulation and click by the user. For example, in addition to measuring the angle, the angle measurement dial 2500 may receive an input or a selection from the user, based on manipulation by the user.

Meanwhile, the angle measurement dial 2500 may be mounted on the body and a passive arm unit of a surgical robot (or a camera robot). The mounting position of the angle measurement dial 2500 may be determined considering the intuitive operating convenience of the user (e.g., an operator or a surgical assistant). For example, the angle measurement dial 2500 may be mounted on an upper portion of the body for facilitating manipulation by the user. As another example, the angle measurement dial 2500 may be mounted on an uppermost portion of the passive arm unit. The mounting position of the angle measurement dial 2500 is not limited to the position illustrated in FIG. 10.

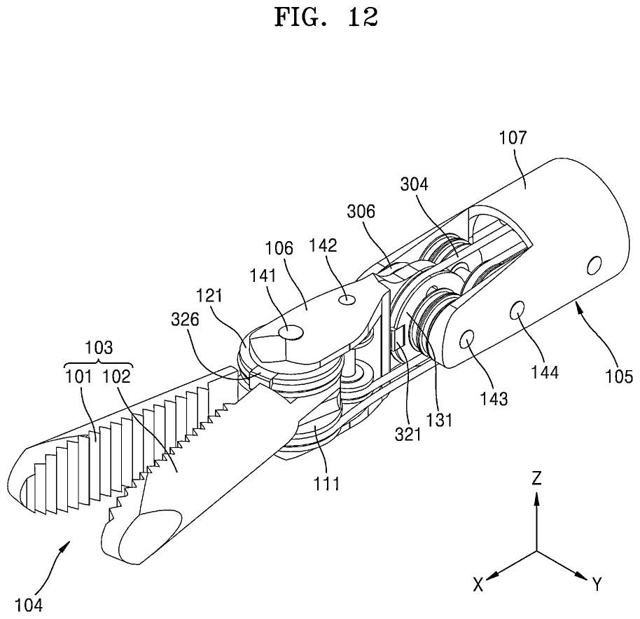

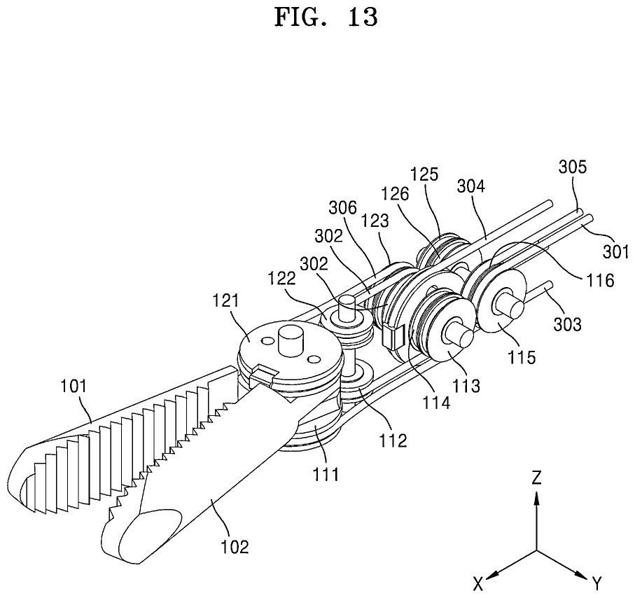

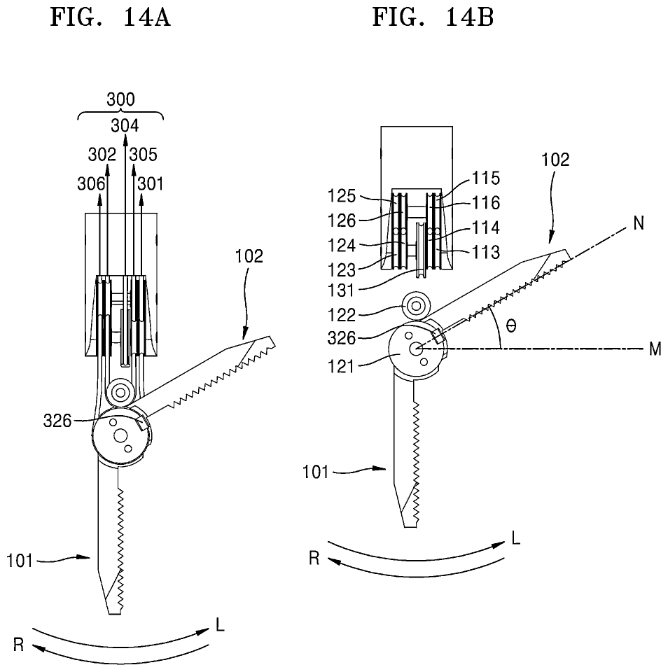

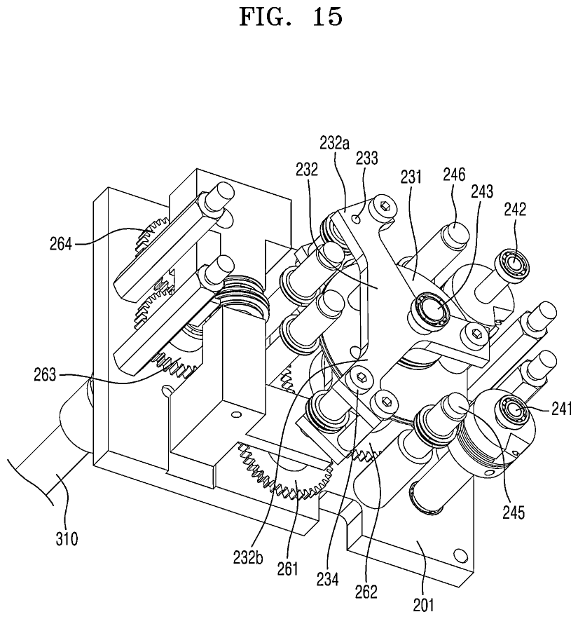

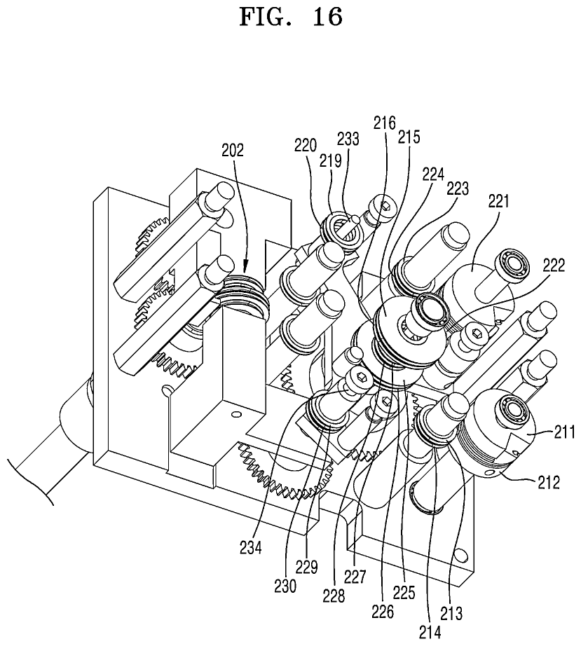

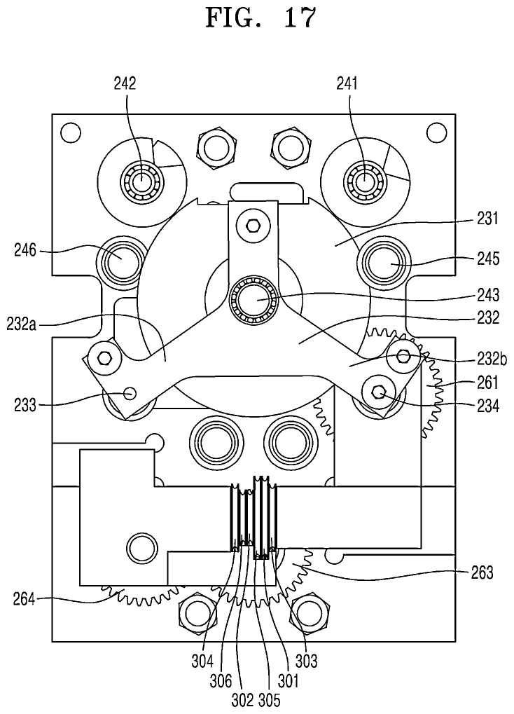

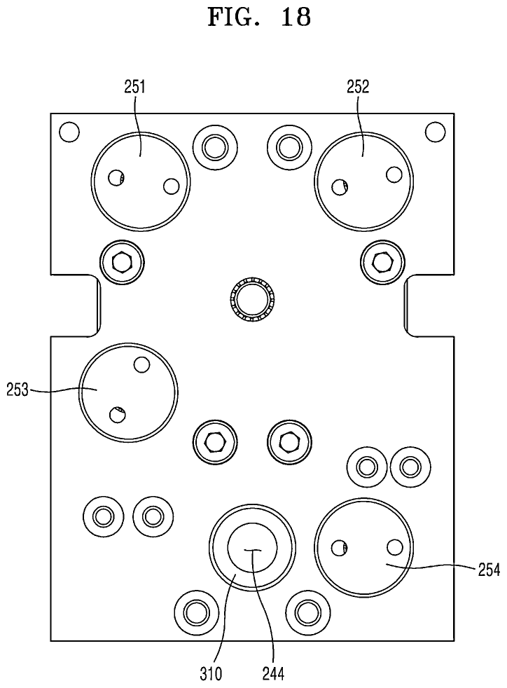

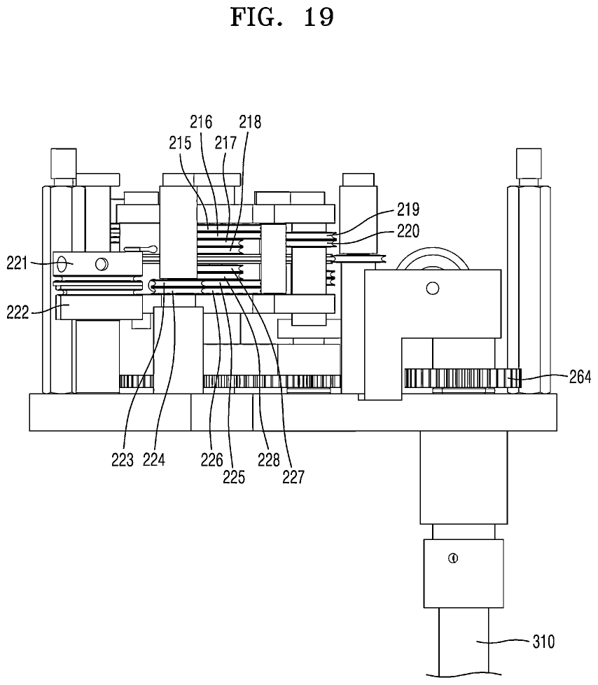

FIG. 11 is a perspective view illustrating a surgical instrument according to an embodiment of the present disclosure, FIGS. 12 and 13 are perspective views of an end tool of the surgical instrument of FIG. 11, and FIGS. 14A and 14B are plan views of the end tool of the surgical instrument of FIG. 11. FIGS. 15 and 16 are perspective views of a driving part of the surgical instrument of FIG. 11, FIG. 17 is a plan view of the driving part of the surgical instrument of FIG. 11, FIG. 18 is a rear view of the driving part of the surgical instrument of FIG. 11, and FIG. 19 is a side view of the driving part of the surgical instrument of FIG. 11.

First, referring to FIG. 11, the surgical instrument 30 according to an embodiment of the present disclosure may include an end tool 100, a driving part 200, and a power transmission part 300, and the power transmission part 300 may include the connection part 310.

The connection part 310 may be formed in the shape of a hollow shaft to accommodate one or more wires (to be described below) therein, and the driving part 200 may be coupled to one end of the connection part 310, and the end tool 100 is coupled to the other end of the connection part 310 such that the connection part 310 serves to connect the driving part 200 to the end tool 100.

The driving part 200 is formed at one end of the connection part 310 and provides an interface that may be coupled to a robotic arm unit (see 21 of FIG. 1 and the like). Thus, when the master robot (see 10 of FIG. 1) is operated by the user, a motor (not shown) of the robotic arm unit (see 21 of FIG. 1 and the like) operates to allow the end tool 100 of the surgical instrument 30 to perform a corresponding motion, and a driving force of the motor (not shown) is transferred to the end tool 100 through the driving part 200. In other words, it may also be described that the driving part 200 itself serves as an interface connecting the surgical instrument 30 to the slave robot 20.

For example, when the user input part (see 13 of FIG. 2) is operated by the user, the motor (not shown) of the robotic arm unit (see 21 of FIG. 1 and the like) operates to allow the end tool 100 of the surgical instrument 30 to perform a corresponding motion, and a driving force of the motor (not shown) may be transferred to the end tool 100 through the driving part 200.

The end tool 100 is formed at the other end of the connection part 310, and is inserted into a surgical site to perform a motion necessary for surgery. As an example of the end tool 100 as described above, a pair of jaws 101 and 102 for performing a grip motion may be used as illustrated in FIG. 12. However, an embodiment of the present disclosure is not limited thereto, and various surgical devices may be used as the end tool 100. For example, a one-armed cautery may be used as the end tool. As the end tool 100 is connected to the driving part 200 by the power transmission part 300, the end tool 100 receives a driving force of the driving part 200 through the power transmission part 300 to perform motions necessary for surgery, such as a grip motion, a cutting motion, or a suturing motion.

Here, the end tool 100 of the surgical instrument 30 according to an embodiment of the present disclosure may be formed to be rotatable in two or more directions, and for example, the end tool 100 may be formed to perform a pitch motion around a rotation shaft 143 of FIG. 12 and simultaneously perform a yaw motion and an actuation motion around a rotation shaft 141 of FIG. 12.

Here, each of the pitch motion, the yaw motion, and the actuation motion used in the present disclosure is defined as follows.

First, the pitch motion refers to a motion of the end tool 100 rotating in a vertical direction with respect to an extension direction of the connection part 310 (the X-axis direction of FIG. 12), that is, a motion of rotating around the Y-axis of FIG. 12. In other words, the pitch motion may refer to a motion that the end tool 100 extending from the connection part 310 in the direction in which the connection part 310 extends (the X-axis direction of FIG. 12) rotates up and down around the Y-axis with respect to the connection part 310.

Next, the yaw motion refers to a motion of the end tool 100 rotating in a horizontal direction, that is, a motion of rotating around the Z-axis of FIG. 12, with respect to the extension direction of the connection part 310 (the X-axis direction of FIG. 12). In other words, the yaw motion may refer to a motion that the end tool 100 extending from the connection part 310 in the direction in which the connection part 310 extends (the X-axis direction of FIG. 12) rotates left and right around the Z-axis with respect to the connection part 310. That is, the yaw motion refers to a motion of the two jaws 101 and 102, which are formed on the end tool 100, rotating around the Z-axis in the same direction.

Meanwhile, the actuation motion refers to a motion of the end tool 100 rotating around the same rotation shaft as that of the yaw motion, but with the two jaws 101 and 102 rotating in the opposite directions to be closed or opened. That is, the actuation motion refers to a motion of the two jaws 101 and 102, which are formed on the end tool 100, rotating around the Z-axis in the opposite directions.

In other words, yaw rotation may refer to a motion of an end tool jaw pulley, which will be described below, rotating around the rotation shaft 141, which is an end tool jaw pulley rotation shaft, and pitch rotation may refer to a motion of the end tool jaw pulley revolving around the rotation shaft 143, which is an end tool pitch rotation shaft.

The roll motion refers to a motion of the surgical instrument rotating around the connection part 310. For example, the roll motion may be a motion of the surgical instrument rotating clockwise or counterclockwise around the extension direction of the connection part 310 (the X-axis direction of FIG. 12).

Meanwhile, the roll motion may refer to a motion of the end tool 100 rotating around the X-axis with respect to the connection part 310. For example, the roll motion may be a motion of the end tool rotating clockwise or counterclockwise around the extension direction of the connection part 310 (the X-axis direction of FIG. 12).

The power transmission part 300 may serve to connect the driving part 200 to the end tool 100 to transfer the driving force of the driving part 200 to the end tool 100, and may include a plurality of wires, pulleys, links, joints, gears, and the like.

Hereinafter, the end tool 100, the driving part 200, the power transmission part 300, and the like of the surgical instrument 30 of FIG. 11 will be described in more detail.

Hereinafter, the power transmission part 300 of the surgical instrument 30 of FIG. 11 will be described in more detail.

Referring to FIGS. 11 to 19, the power transmission part 300 of the surgical instrument according to an embodiment of the present disclosure may include a wire 301, a wire 302, a wire 303, a wire 304, a wire 305, and a wire 306.

Here, the wire 301 and the wire 305 may be paired to serve as a first jaw wire. The wire 302 and the wire 306 may be paired to serve as a second jaw wire. Here, a component encompassing the wire 301 and the wire 305, which constitute the first jaw wire, and the wire 302 and the wire 306, which constitute the second jaw wire, may be referred to as a jaw wire. In addition, the wire 303 and the wire 304 may be paired to serve as a pitch wire.

Here, the drawing illustrates that a pair of wires are associated with a rotational motion of the first jaw 101, and another pair of wires are associated with a rotational motion of the second jaw 102, but an embodiment of the present disclosure is not limited thereto. For example, a pair of wires may be associated with the yaw motion, and another pair of wires may be associated with the actuation motion.

In addition, the power transmission part 300 of the surgical instrument 30 according to an embodiment of the present disclosure may include a coupling member 321, a coupling member 326, and the like, which are coupled to ends of the respective wires to couple the wires to pulleys. Here, each of the coupling members may have various shapes as necessary, such as a ball shape, or a tube shape.

Here, the coupling member 321, which is a pitch wire coupling member, may be coupled to ends of the wire 303 and the wire 304, which constitute the pitch wire, on the side of the end tool 100, to serve as a pitch wire-end tool coupling member. Meanwhile, although not illustrated in the drawings, a pitch wire-driving part coupling member (not shown) may be coupled to ends of the wire 303 and the wire 304, which constitute the pitch wire, on the side of the driving part 200.

Meanwhile, the coupling member 326, which is a second jaw wire coupling member, may be coupled to ends of the wire 302 and the wire 306, which constitute the second jaw wire, on the side of the end tool 100, to serve as a second jaw wire-end tool coupling member. Meanwhile, although not illustrated in the drawings, a second jaw wire-driving part coupling member (not shown) may be coupled to ends of the wire 302 and the wire 306, which constitute the second jaw wire, on the side of the driving part 200.

Meanwhile, although not illustrated in the drawings, a coupling member (not shown) having the same shape as the coupling member 326 may be coupled to ends of the wire 301 and the wire 305, which constitute the first jaw wire, on the side of the end tool 100, to serve as a first jaw wire-end tool coupling member. Meanwhile, although not illustrated in the drawings, a first jaw wire-driving part coupling member (not shown) may be coupled to ends of the wire 301 and the wire 305, which constitute the first jaw wire, on the side of the driving part 200.

Here, the coupling members are classified as being included in the power transmission part 300, but the coupling members on the side of the end tool 100 may be classified as being included in the end tool 100, and the coupling members on the side of the driving part 200 may be classified as being included in the driving part 200.

The coupling relationship between the wires, the coupling members, and each pulley may be described in detail as follows.

First, the wire 302 and the wire 306, which constitute the second jaw wire, may be a single wire. When the coupling member 326, which is a second jaw wire-end tool coupling member, is fit into a middle point of the second jaw wire, and the coupling member 326 is fixed through crimping, both strands of the second jaw wire around the coupling member 326 may be referred to as the wire 302 and the wire 306, respectively.

Alternatively, the wire 302 and the wire 306, which constitute the second jaw wire, may be formed as separate wires and connected to each other by the coupling member 326.

In addition, by coupling the coupling member 326 to a pulley 121, the wire 302 and the wire 306 may be fixedly coupled to the pulley 121. Accordingly, the pulley 121 may be rotated as the wire 302 and the wire 306 are pulled and released.

A second jaw wire-driving part coupling member (not shown) may be coupled to ends of the wire 302 and the wire 306 that are opposite to the ends to which the coupling member 326 is coupled. That is, by fitting the opposite ends of the wire 302 and the wire 306 into the second jaw wire-driving part coupling member (not shown), and then crimping the coupling member (not shown), each of the wire 302 and the wire 306 may be fixed to the second jaw wire-driving part coupling member (not shown).

In addition, by coupling the second jaw wire-driving part coupling member (not shown), which is coupled to the wire 302 and the wire 306, to each of a pulley 221 and a pulley 222, the wire 302 and the wire 306 may be fixedly coupled to the pulley 221 and the pulley 222, respectively. Accordingly, when the pulley 221 and the pulley 222 is rotated by a motor or a human force, the pulley 121 of the end tool 100 may be rotated as the wire 302 and the wire 306 are pulled and released.

Here, a driving part second jaw pulley may include two pulleys, i.e., the pulley 221 and the pulley 222, and thus, the second jaw wire-driving part coupling member may also include two coupling members. Alternatively, the driving part second jaw pulley may include one pulley, the second jaw wire-driving part coupling member may also include one coupling member, and the wire 302 and the wire 306 may be coupled to one coupling member and thus coupled to one driving part second jaw pulley.

In the same manner, the wire 301 and the wire 305, which constitute the first jaw wire, are coupled to the first jaw wire-end tool coupling member (not shown) and the first jaw wire-driving part coupling member (not shown), respectively. In addition, the first jaw wire-end tool coupling member (not shown) is coupled to a pulley 111, and the first jaw wire-driving part coupling member (not shown) is coupled to a pulley 211 and a pulley 212. Accordingly, when the pulley 211 and the pulley 212 is rotated by a motor or a human force, the pulley 111 of the end tool 100 may be rotated as the wire 301 and the wire 305 are pulled and released.

In the same manner, ends of the wire 303 and the wire 304, which constitute the pitch wire, are coupled to the coupling member 321, which is the pitch wire-end tool fastening member, and the other ends of the wire 303 and the wire 304 are coupled to the pitch wire-driving part coupling member (not shown). In addition, the coupling member 321 is coupled to a pulley 131, and the pitch wire-driving part coupling member (not shown) is coupled to a pulley 231. Accordingly, when the pulley 231 is rotated by a motor or a human force, the pulley 131 of the end tool 100 may be rotated as the wire 303 and the wire 304 are pulled and released.

Accordingly, the wire 301 and the wire 305, which are both strands of the first jaw wire, may be coupled to a coupling member 323, which is the first jaw wire-end tool coupling member, and the first jaw wire-driving part coupling member (not shown), so as to form a closed loop together. Similarly, each of the second jaw wire and the pitch wire may be formed to form a closed loop.

Hereinafter, the end tool 100 of the surgical instrument 30 of FIG. 11 will be described in more detail.

FIGS. 12 and 13 are perspective views illustrating the end tool of the surgical instrument of FIG. 11, and FIGS. 14A and 14B are plan views illustrating the end tool of the surgical instrument of FIG. 11. Here, FIG. 12 illustrates a state in which an end tool hub 106 and a pitch hub 107 are coupled to each other, and FIG. 13 illustrates a state in which the end tool hub 106 and the pitch hub 107 are removed.

Referring to FIGS. 12, 13, and 14, the end tool 100 of an embodiment of the present disclosure includes a pair of jaws for performing a grip motion, that is, the first jaw 101 and the second jaw 102. Here, a component encompassing each of the first jaw 101 and the second jaw 102 or both the first jaw 101 and the second jaw 102 may be referred to as a jaw 103.

In addition, the end tool 100 may include the pulley 111, a pulley 112, a pulley 113, a pulley 114, a pulley 115, and a pulley 116, which are associated with a rotational motion of the first jaw 101. In addition, the end tool 100 may include the pulley 121, a pulley 122, a pulley 123, a pulley 124, a pulley 125, and a pulley 126, which are associated with a rotational motion of the second jaw 102.

Here, the drawings illustrate that a group of pulleys are associated with the rotational motion of the first jaw 101, and another group of pulleys are associated with the rotational motion of the second jaw 102, but an embodiment of the present disclosure is not limited thereto. For example, one group of pulleys within the end tool may be associated with a yaw motion, and another group of pulleys may be associated with an actuation motion. Here, the pulleys included in the end tool 100, including the pulleys described above, may be collectively referred to as an end tool pulley.

Meanwhile, although the drawings illustrate that the pulleys facing each other are arranged in parallel with each other, an embodiment of the present disclosure is not limited thereto, and the pulleys may be formed in various positions and sizes suitable for the configuration of the end tool.

In addition, the end tool 100 of an embodiment of the present disclosure may include the end tool hub 106 and the pitch hub 107.

The rotation shaft 141 and a rotation shaft 142, which will be described below, may be inserted through the end tool hub 106, and the end tool hub 106 may accommodate therein at least portions of the first jaw 101 and the second jaw 102, which are axially coupled to the rotation shaft 141. In addition, the end tool hub 106 may accommodate therein at least portions of the pulley 112 and the pulley 122, which are axially coupled to the rotation shaft 142.

In addition, the pulley 131 serving as an end tool pitch pulley may be formed at one end of the end tool hub 106. As illustrated in FIG. 12, the pulley 131 may be formed as a separate member from the end tool hub 106, and coupled to the end tool hub 106. Alternatively, although not illustrated in the drawings, the pulley 131 may be formed with the end tool hub 106 as one body. That is, one end of the end tool hub 106 may be formed in a disk shape or a semicircular shape like a pulley, and a groove around which a wire may be wound may be formed on an outer circumferential surface of the end tool hub 106. The wire 303 and the wire 304 described above may be coupled to the pulley 131 serving as an end tool pitch pulley, and a pitch motion may be performed as the pulley 131 is rotated around the rotation shaft 143.

The rotation shaft 143 and a rotation shaft 144, which will be described below, may be inserted through the pitch hub 107, and the pitch hub 107 may be axially coupled to the end tool hub 106 and the pulley 131 by the rotation shaft 143. Thus, the end tool hub 106 and the pulley 131 (coupled to the end tool hub 106) may be formed to be rotatable around the rotation shaft 143 with respect to the pitch hub 107.

In addition, the pitch hub 107 may accommodate therein at least portions of the pulley 113, the pulley 114, the pulley 123, and the pulley 124, which are axially coupled to the rotation shaft 143. In addition, the pitch hub 107 may accommodate therein at least portions of the pulley 115, the pulley 116, the pulley 125, and the pulley 126, which are axially coupled to the rotation shaft 144.

Further, the end tool 100 of an embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 106, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

One or more pulleys may be fit into each of the rotation shafts 141, 142, 143, and 144, which will be described in detail below.

The pulley 111 functions as an end tool first jaw pulley, the pulley 121 functions as an end tool second jaw pulley, and these two components may be collectively referred to as an end tool jaw pulley.

The pulley 111 and the pulley 121, which constitute the end tool jaw pulley, are formed to face each other, and are formed to be rotatable independently of each other around the rotation shaft 141, which is an end tool jaw pulley rotation shaft. Here, the drawings illustrate that the pulley 111 and the pulley 121 are formed to be rotated around one rotation shaft 141, but it is needless to say that each jaw pulley may be formed to be rotatable around a separate shaft. Here, the first jaw 101 may be fixedly coupled to the pulley 111 to be rotated together with the pulley 111, and the second jaw 102 may be fixedly coupled to the pulley 121 to be rotated together with the pulley 121. A yaw motion and an actuation motion of the end tool 100 are performed according to rotation of the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be formed as one body. Similarly, the second jaw 102 and the pulley 121 may be formed as separate members and coupled to each other, or the second jaw 102 and the pulley 121 may be formed as one body.

The pulley 112 functions as an end tool first jaw auxiliary pulley, the pulley 122 functions as an end tool second jaw auxiliary pulley, and these two components may be collectively referred to as an end tool jaw auxiliary pulley.

In detail, the pulley 112 and the pulley 122, which constitute the end tool jaw auxiliary pulley, may be additionally provided on one side of the pulley 111 and the pulley 121. In other words, the pulley 112, which is an auxiliary pulley, may be arranged between the pulley 111 and the pulley 113/pulley 114. In addition, the pulley 122, which is an auxiliary pulley, may be arranged between the pulley 121 and the pulley 123/pulley 124. The pulley 112 and the pulley 122 may be formed to be rotatable independently of each other around the rotation shaft 142. Here, the drawings illustrate that the pulley 112 and the pulley 122 are formed to be rotated around one rotation shaft 142, but it is needless to say that the pulley 112 and the pulley 122 may be formed to be rotatable around separate shafts, respectively. The auxiliary pulleys will be described in more detail below.

The pulley 113 and the pulley 114 may function as an end tool first jaw pitch main pulley, the pulley 123 and the pulley 124 may function as an end tool second jaw pitch main pulley, and these two components may collectively be referred to as an end tool jaw pitch main pulley.

The pulley 115 and the pulley 116 may function as an end tool first jaw pitch sub-pulley, the pulley 125 and the pulley 126 may function as an end tool second jaw pitch sub-pulley, and these two components may collectively be referred to as an end tool jaw pitch sub-pulley.

Hereinafter, components associated with rotation of the pulley 111 will be described.

The pulley 113 and the pulley 114 may function as an end tool first jaw pitch main pulley. That is, the pulley 113 and the pulley 114 function as a main rotation pulley for a pitch motion of the first jaw 101. Here, the wire 301, which constitutes the first jaw wire, is wound around the pulley 113, and the wire 305, which constitutes the first jaw wire, is wound around the pulley 114.

The pulley 115 and the pulley 116 function as an end tool first jaw sub-pulley. That is, the pulley 115 and the pulley 116 function as a sub-rotation pulley for a pitch motion of the first jaw 101. Here, the wire 301, which constitutes the first jaw wire, is wound around the pulley 115, and the wire 305, which constitutes the first jaw wire, is wound around the pulley 116.

Here, on one side of the pulley 111 and the pulley 112, the pulley 113 and the pulley 114 are arranged to face each other. Here, the pulley 113 and the pulley 114 are formed to be rotatable independently of each other around the rotation shaft 143, which is an end tool pitch rotation shaft. In addition, the pulley 115 and the pulley 116 are arranged on one sides of the pulley 113 and the pulley 114, respectively, to face each other. Here, the pulley 115 and the pulley 116 are formed to be rotatable independently of each other around the rotation shaft 144, which is an end tool pitch auxiliary rotation shaft. Here, the drawings illustrate that the pulley 113, the pulley 115, the pulley 114, and the pulley 116 are formed to be rotatable around the Y-axis direction, but an embodiment of the present disclosure is not limited thereto, and the rotation shafts of the respective pulleys may be formed in various directions suitable for their configurations.

The wire 301, which constitutes the first jaw wire, is wound to sequentially come into contact with at least portions of the pulley 115, the pulley 113, and the pulley 111. In addition, the wire 305 connected to the wire 301 by the coupling member 323 is wound to sequentially come into contact with at least portions of the pulley 111, the pulley 112, the pulley 114, and the pulley 116.

In other words, the wire 301 and the wire 305, which constitute first jaw wire, are wound to sequentially come into contact with at least portions of the pulley 115, the pulley 113, the pulley 111, the pulley 112, the pulley 114, and the pulley 116, and are formed to move along the above pulleys while rotating the above pulleys.

Accordingly, when the wire 301 is pulled in the direction of an arrow 301 of FIG. 14A, the coupling member (not shown) to which the wire 301 is coupled and the pulley 111 coupled to the coupling member (not shown) are rotated in the direction of an arrow L of FIG. 14A. On the contrary, when the wire 305 is pulled in the direction of an arrow 305 of FIG. 14A, the coupling member (not shown) to which the wire 305 is coupled and the pulley 111 coupled to the coupling member (not shown) are rotated in the direction of an arrow R of FIG. 14A.

Hereinafter, the pulley 112 and the pulley 122 serving as auxiliary pulleys will be described in more detail.

The pulley 112 and the pulley 122 may come into contact with the wire 305, which constitutes the first jaw wire, and the wire 302, which constitutes the second jaw wire to change the arrangement path of the wire 305 and the wire 302 to a certain extent, and thus perform a function of increasing a rotation angle of each of the first jaw 101 and the second jaw 102.

That is, when no auxiliary pulley is arranged, each of the first jaw and the second jaw may be rotated up to the right angle, however, in an embodiment of the present disclosure, by additionally arranging the pulley 112 and the pulley 122, which are auxiliary pulleys, the maximum rotation angle may be increased by θ as illustrated in FIG. 14B. This enables an opening motion of the two jaws of the end tool 100 for an actuation motion in a state in which the two jaws are yaw-rotated by 90° in the L direction. This is because the second jaw 102 may be rotated by the additional angle θ as illustrated in FIG. 12. Similarly, the actuation motion may be performed even in a state in which the two jaws are yaw-rotated in the R direction. In other words, a feature of increasing the range of yaw rotation in which an actuation motion is possible may be obtained through the pulley 112 and the pulley 122.

This will be described in more detail as follows.

When no auxiliary pulley is arranged, as the first jaw wire is fixedly coupled to the end tool first jaw pulley, and the second jaw wire is fixedly coupled to the end tool second jaw pulley, each of the end tool first jaw pulley and the end tool second jaw pulley may rotate only up to 90°. In this case, when an actuation motion is performed in a state in which the first jaw and the second jaw are located at a 90° line, the first jaw may be opened, but the second jaw may not be rotated beyond 90°. Thus, there was a problem that, in a state in which the first jaw and the second jaw perform a yaw motion over a certain angle, the actuation motion is not smoothly performed.

In order to address such a problem, in the surgical instrument 30 according to an embodiment of the present disclosure, the pulley 112 and the pulley 122, which are auxiliary pulleys, are additionally arranged on one sides of the pulley 111 and the pulley 121, respectively. As described above, as the arrangement paths of the wire 305, which constitutes the first jaw wire, and the wire 302, which constitutes the second jaw wire, are changed to a certain extent by arranging the pulley 112 and the pulley 122, a tangential direction of the wire 305 and the wire 302 is changed, and accordingly, the coupling member 326 for coupling the wire 302 and the pulley 121 may be rotated up to a line N of FIG. 14B. That is, the coupling member 326, which is a coupling part of the wire 302 and the pulley 121, is rotatable until the coupling member 326 is located on a common internal tangent of the pulley 121 and the pulley 122. Similarly, the coupling member 323, which is a coupling part of the wire 305 and the pulley 111, is rotatable until the coupling member 323 is located on a common internal tangent of the pulley 111 and the pulley 112, such that the range of rotation in the L direction may be increased.

In other words, the wire 301 and the wire 305, which are two strands of the first jaw wire wound around the pulley 111 by the pulley 112, are arranged at one side with respect to a plane perpendicular to the Y-axis and passing through the X-axis. Simultaneously, the wire 302 and the wire 306, which are two strands of the second jaw wire wound around the pulley 121 by the pulley 122, are arranged at the other side with respect to the plane perpendicular to the Y-axis and passing through the X-axis.

In other words, the pulley 113 and the pulley 114 are arranged at one side with respect to the plane perpendicular to the Y-axis and passing through the X-axis, and the pulley 123 and the pulley 124 are arranged at the other side with respect to the plane perpendicular to the Y-axis and passing through the X-axis.

In other words, the wire 305 is located on the internal tangent of the pulley 111 and the pulley 112, and the rotation angle of the pulley 111 is increased by the pulley 112. In addition, the wire 302 is located on the internal tangent of the pulley 121 and the pulley 122, and the rotation angle of the pulley 121 is increased by the pulley 122.

According to an embodiment of the present disclosure, as the radii of rotation of the jaw 101 and the jaw 102 increase, an effect of increasing a yaw motion range in which a normal opening/closing actuation motion is performed may be obtained.

Next, components associated with rotation of the pulley 121 will be described.

The pulley 123 and the pulley 124 function as an end tool second jaw pitch main pulley. That is, the pulley 123 and the pulley 124 function as main rotation pulleys for a pitch motion of the second jaw 102. Here, the wire 306, which constitutes the second jaw wire, is wound around the pulley 123, and the wire 302, which constitutes the second jaw wire, is wound around the pulley 124.

The pulley 125 and the pulley 126 function as an end tool second jaw sub-pulley. That is, the pulley 125 and the pulley 126 function as sub-rotation pulleys for a pitch motion of the second jaw 102. Here, the wire 306, which constitutes the second jaw wire, is wound around the pulley 125, and the wire 302, which constitutes the second jaw wire, is wound around the pulley 126.

On one side of the pulley 121, the pulley 123 and the pulley 124 are arranged to face each other. Here, the pulley 123 and the pulley 124 are formed to be rotatable independently of each other around the rotation shaft 143, which is an end tool pitch rotation shaft. In addition, the pulley 125 and the pulley 126 are arranged on one sides of the pulley 123 and the pulley 124, respectively, to face each other. Here, the pulley 125 and the pulley 126 are formed to be rotatable independently of each other around the rotation shaft 144, which is an end tool pitch auxiliary rotation shaft. Here, the drawings illustrate that the pulley 123, the pulley 125, the pulley 124, and the pulley 126 are all formed to be rotatable around the Y-axis direction, but an embodiment of the present disclosure is not limited thereto, and the rotation shafts of the respective pulleys may be formed in various directions suitable for their configurations.

The wire 306, which constitutes the second jaw wire, is wound to sequentially come into contact with at least portions of the pulley 125, the pulley 123, and the pulley 121. In addition, the wire 302 connected to the wire 306 by the coupling member 326 is wound to sequentially come into contact with at least portions of the pulley 121, the pulley 122, the pulley 124, and the pulley 126.

In other words, the wire 306 and the wire 302, which constitute the second jaw wire, are wound to sequentially come into contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of the arrow 306 of FIG. 14A, the coupling member 326 to which the wire 306 is coupled and the pulley 121 coupled to the coupling member 326 are rotated in the direction of the arrow R of FIG. 14A. On the contrary, when the wire 302 is pulled in the direction of an arrow 302 of FIG. 14A, the coupling member 326 to which the wire 302 is coupled and the pulley 121 coupled to the coupling member 326 are rotated in the direction of the arrow L of FIG. 14A.

Hereinafter, a pitch motion of the present disclosure will be described in more detail.

First, for a pitch motion, on the side of end tool 100, the pulley 113, the pulley 114, the pulley 123, and the pulley 124, which constitute the end tool jaw pitch main pulley, are formed to be rotatable around the rotation shaft 143. Meanwhile, on the side of the proximal end 105 of the end tool jaw pitch main pulley, the pulley 115, the pulley 116, the pulley 125, and the pulley 126, which constitute the end tool jaw pitch sub-pulley, are formed to be rotatable around the rotation shaft 144.

In addition, with respect to a plane (i.e., an XY plane) perpendicular to the rotation shaft 141 and including the rotation shaft 143, the wire 301 and the wire 305, which are two strands of the first jaw wire, are located on the same side with respect to the XY plane. That is, the wire 301 and the wire 305 are formed to pass through the lower sides of the pulley 114 and the pulley 113, which constitute the end tool jaw pitch main pulley, and the upper sides of the pulley 115 and pulley 116, which constitute the end tool jaw pitch sub-pulley.

Similarly, the wire 302 and the wire 306, which are two strands of the second jaw wire, are located on the same side with respect to the XY plane. That is, the wire 302 and the wire 306 are formed to pass through the upper sides of the pulley 123 and the pulley 124, which constitute the end tool jaw pitch main pulley, and the lower sides of the pulley 125 and pulley 126, which constitute the end tool jaw pitch sub-pulley.

In addition, for the wire 301 and the wire 305 that are two strands of the first jaw wire, when the wire 301 is pulled in the direction of the arrow 301 of FIG. 14A, and simultaneously the wire 305 is pulled in the direction of the arrow 305 of FIG. 14A (i.e., when both strands of the first jaw wire are pulled in the same direction), the wire 301 and the wire 305 are wound below the pulley 113 and the pulley 114 that are rotatable around the rotation shaft 143 that is the end tool pitch rotation shaft as illustrated in FIG. 12, thus, the pulley 111 to which the wire 301 and the wire 305 are fixedly coupled, and the end tool hub 106 to which the pulley 111 is coupled are rotated together counterclockwise around the rotation shaft 143, and accordingly, the end tool 100 rotates downward to perform a pitch motion. At this time, because the second jaw 102 and the wire 302 and the wire 306 both fixedly coupled to the second jaw 102 are wound around upper portions of the pulley 123 and the pulley 124 that are rotatable around the rotation shaft 143, the wire 302 and the wire 306 are unwound in the opposite directions of the arrows 302 and 306, respectively.

On the contrary, for the wire 302 and the wire 306 that are two strands of the second jaw wire, when the wire 302 is pulled in the direction of the arrow 302 of FIG. 14A, and simultaneously the wire 306 is pulled in the direction of the arrow 306 of FIG. 14A (i.e., when both strands of the second jaw wire are pulled in the same direction), the wire 302 and the wire 302 are wound above the pulley 123 and the pulley 124 that are rotatable around the rotation shaft 143 that is the end tool pitch rotation shaft as illustrated in FIG. 12, thus, the pulley 121 to which the wire 302 and the wire 306 are fixedly coupled, and the end tool hub 106 to which the pulley 121 is coupled are rotated together clockwise around the rotation shaft 143, and accordingly, the end tool 100 rotates upward to perform a pitch motion. At this time, because the first jaw 101 and the wire 301 and the wire 305 both fixedly coupled to the first jaw 101 are wound around lower portions of the pulley 113 and the pulley 114 that are rotatable around the rotation shaft 143, the wire 302 and the wire 306 are moved in the opposite directions of the arrows 301 and 305, respectively.

In other words, it may also be described that, when pitch rotation of the end tool 100 is performed, both strands of each jaw wire move simultaneously in the same direction.

Meanwhile, the end tool 100 of the instrument 30 of the present disclosure may further include the pulley 131, which is an end tool pitch pulley, the driving part 200 may further include the pulley 231, which is a driving part pitch pulley, and the power transmission part 300 further include the wire 303 and the wire 304 that are pitch wires. In detail, the pulley 131 of the end tool 100 is rotatable around the rotation shaft 143, which is an end tool pitch rotation shaft, and may be formed with the end tool hub 106 as one body (or to be fixedly coupled to the end tool hub 106). In addition, the wire 303 and the wire 304 may serve to connect the pulley 131 of the end tool 100 to the pulley 231 of the driving part 200.

Thus, when the pulley 231 of the driving part 200 rotates, the rotation of the pulley 231 is transferred to the pulley 131 of the end tool 100 through the wire 303 and the wire 304 such that the pulley 131 is rotated together, and accordingly, the end tool 100 rotates to perform a pitch motion.

The instrument 30 according to an embodiment of the present disclosure includes the pulley 131 of the end tool 100, the pulley 231 of the driving part 200, and the wire 303 and the wire 304 of the power transmission part 300, and thus allowing a driving force of a pitch motion of the driving part 200 to be more completely transferred to the end tool 100, thereby improving the operational reliability.

Here, the diameter of each of the pulley 113, the pulley 114, the pulley 123, and the pulley 124, which constitute the end tool jaw pitch main pulley, may be equal to or different from the diameter of the pulley 131, which is an end tool pitch pulley. Here, the ratio of the diameter of the end tool jaw pitch main pulley to the diameter of the end tool pitch pulley may be equal to the ratio of the diameter of a driving part relay pulley of the driving part 200, which will be described below, to the diameter of the driving part pitch pulley. This will be described in detail below.

Hereinafter, the driving part 200 of the instrument 30 of FIG. 11 will be described in more detail.

Referring to FIGS. 15 to 21, the driving part 200 of the instrument 30 according to an embodiment of the present disclosure may include the pulley 211, the pulley 212, a pulley 213, a pulley 214, a pulley 215, a pulley 216, a pulley 217, a pulley 218, a pulley 219, and a pulley 220, which are associated with a rotational motion of the first jaw 101. In addition, the driving part 200 may include the pulley 221, the pulley 222, a pulley 223, a pulley 224, a pulley 225, a pulley 226, a pulley 227, a pulley 228, a pulley 229, and a pulley 230, which are associated with a rotational motion of the second jaw 102.

Here, although the drawings illustrate that the pulleys facing each other are arranged in parallel with each other, an embodiment of the present disclosure is not limited thereto, and the pulleys may be formed in various positions and sizes suitable for the configuration of the driving part.

In addition, the driving part 200 of the instrument 30 according to an embodiment of the present disclosure may further include the pulley 231 serving as a driving part pitch pulley, and a pitch-yaw connector 232 connecting the pulley 231 to the above-described driving part jaw pulleys.

In addition, the driving part 200 of an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a driving part first jaw rotation shaft, and the rotation shaft 242 may function as a driving part second jaw rotation shaft. In addition, the rotation shaft 243 may function as a driving part pitch rotation shaft, and the rotation shaft 244 may function as a driving part roll rotation shaft. In addition, the rotation shaft 245 may function as a driving part first jaw auxiliary rotation shaft, and the rotation shaft 246 may function as a driving part second jaw auxiliary rotation shaft. One or more pulleys may be fit into each of the rotation shafts 241, 242, 243, 244, 245, and 246, and this will be described in detail below.

In addition, the driving part 200 of an embodiment of the present disclosure may include a motor coupling part 251, a motor coupling part 252, a motor coupling part 253, and a motor coupling part 254. Here, the motor coupling part 251 may function as a first jaw driving motor coupling part, the motor coupling part 252 may function as a second jaw driving motor coupling part, the motor coupling part 253 may function as a pitch driving motor coupling part, and the motor coupling part 254 may function as a roll driving motor coupling part. Here, each of the motor coupling parts 251, 252, 253, and 254 may be formed in the form of a rotatable flat plate, and may have formed thereon one or more coupling holes to which a motor (not shown) may be coupled.

The motor coupling parts 251, 252, 253, and 254 of the driving part 200 as described above are coupled to motors (not shown) formed in the robotic arm units 21, 22, and 23, respectively, such that the driving part 200 is operated by driving of the motors (not shown).

In addition, the driving part 200 of an embodiment of the present disclosure may include a gear 261, a gear 262, a gear 263, and a gear 264. Here, the gear 261 and the gear 262 may function as pitch driving gears, and the gear 263 and the gear 264 may function as roll driving gears.

Hereinafter, each component will be described in more detail.

The pulley 211 and the pulley 212 may function as driving part first jaw pulleys, the pulley 221 and the pulley 222 may function as driving part second jaw pulleys, and these components may be collectively referred to as a driving part jaw pulley.

Here, the drawings illustrate that the pulley 211 is associated with a rotational motion of the first jaw 101 of the end tool 100, and the pulley 221 is associated with a rotational motion of the second jaw 102 of the end tool 100, but an embodiment of the present disclosure is not limited thereto. For example, one group of pulleys within the driving part may be associated with a yaw motion, and another group of pulleys may be associated with an actuation motion. Thus, the pulley 211 and the pulley 212 may be collectively referred to as a driving part driving pulley. In addition, in the other pulleys, one group of pulleys may be associated with a yaw motion, and another group of pulleys may be associated with an actuation motion.

The pulley 213 and the pulley 214 may function as driving part first jaw auxiliary pulleys, the pulley 223 and the pulley 224 may function as driving part second jaw auxiliary pulleys, and these components may be collectively referred to as a driving part auxiliary pulley.

The pulley 215 and the pulley 216 may function as driving part first jaw first relay pulleys, the pulley 217 and the pulley 218 may function as driving part first jaw second relay pulleys, and these components may be collectively referred to as a driving part first jaw relay pulley. Meanwhile, the pulley 225 and the pulley 226 may function as driving part second jaw first relay pulleys, the pulley 227 and the pulley 228 may function as driving part second jaw second relay pulleys, and these components may be collectively referred to as a driving part second jaw relay pulley. Meanwhile, the pulley 215, the pulley 216, the pulley 225, and the pulley 226 may be collectively referred to as a driving part first relay pulley, and the pulley 217, the pulley 218, the pulley 227, and the pulley 228 may be collectively referred to as a driving part second relay pulley. Furthermore, the pulley 215, the pulley 216, the pulley 217, the pulley 218, the pulley 225, the pulley 226, the pulley 227, and the pulley 228 may be collectively referred to as a driving part relay pulley.

Here, the drawings illustrate that two pulleys are paired to constitute the driving part relay pulley for each jaw, but an embodiment of the present disclosure is not limited thereto. For example, it is illustrated that the pulley 215, which is a driving part first jaw first relay pulley, and the pulley 217, which is a driving part first jaw second relay pulley, are formed as a pair, and the wire 301 sequentially passes through the pulley 215 and the pulley 217. However, the driving part first jaw relay pulley may be configured with three or more pulleys rather than with two pulleys.

Meanwhile, the pulley 219 and the pulley 220 may function as driving part first jaw satellite pulleys, the pulley 229 and the pulley 230 may function as driving part second jaw satellite pulleys, and these two components may be collectively referred to as a driving part satellite pulley.

A plurality of rotation shafts including the rotation shaft 241, the rotation shaft 242, the rotation shaft 243, the rotation shaft 244, the rotation shaft 245, and the rotation shaft 246 may be formed on a first surface of a base plate 201. In addition, a plurality of relay pulleys 202 may be formed on the first surface of the base plate 201, and may serve to redirect, toward the pulley 231, the wires 301, 302, 303, 304, 305, and 306 entering the driving part 200 through the connection part 310.

In addition, the connection part 310 in the form of a shaft may be coupled to a second surface of the base plate 201 opposite to the first surface, and the motor coupling part 251, the motor coupling part 252, the motor coupling part 253, and the motor coupling part 254, to which the motors (not shown) for driving the pulleys are coupled, may be formed on the second surface.

Here, each rotation shaft and each motor coupling part may be directly connected or indirectly connected via a gear.

For example, by directly coupling the motor coupling part 251, which is a first jaw driving motor coupling part, to the rotation shaft 241, which is a driving part first jaw rotation shaft, when the motor coupling part 251 coupled to a first jaw driving motor (not shown) is rotated, the rotation shaft 241 directly coupled to the motor coupling part 251 may be rotated together. Similarly, by directly coupling the motor coupling part 252, which is a second jaw driving motor coupling part, to the rotation shaft 242, which is a driving part second jaw rotation shaft, when the motor coupling part 252 coupled to a second jaw driving motor (not shown) is rotated, the rotation shaft 242 directly coupled to the motor coupling part 252 may be rotated together.

As another example, when viewed from a plane perpendicular to the rotation shaft 243, the motor coupling part 253, which is a pitch driving motor coupling part, and the rotation shaft 243, which is a driving part pitch rotation shaft, may be arranged to be spaced apart from each other by a certain extent. In addition, the motor coupling part 253 and the rotation shaft 243 may be connected to each other by the gear 261 and the gear 263, which are pitch driving gears.

Similarly, when viewed from a plane perpendicular to the rotation shaft 244, the motor coupling part 254, which is a roll driving motor coupling part, and the rotation shaft 244, which is a driving part roll rotation shaft, may be arranged to be spaced apart from each other by a certain extent. In addition, the motor coupling part 254 and the rotation shaft 244 may be connected to each other by the gear 263 and the gear 264, which are roll driving gears.

As such, some motor coupling parts are configured to be directly connected to the rotation shafts, respectively, and the other motor coupling parts are configured to be indirectly connected to the rotation shafts, respectively, because the coupling position and direction between the instrument 30 and the slave robot 20 need to be considered. That is, the rotation shaft that is not affected by the coupling position with the slave robot 20 may be directly connected to the motor coupling part, whereas the rotation shaft that may cause interference with the coupling position with the slave robot 20 may be indirectly connected to the motor coupling part.

The drawings illustrate that the motor coupling part 251 and the motor coupling part 252 are directly connected to the rotation shafts, and the motor coupling part 253 and the motor coupling part 254 are indirectly connected to the rotation shafts through the gears, but an embodiment of the present disclosure is not limited thereto, and various configurations are possible according to the coupling position and direction with the slave robot 20.

The pulley 211 and the pulley 212, which are driving part first jaw pulleys, may be coupled to the rotation shaft 241, which is a driving part first jaw rotation shaft. Here, the pulley 211 and the pulley 212 may be formed to rotate together with the rotation shaft 241.

In addition, the rotation shaft 245, which is a driving part first jaw auxiliary rotation shaft, may be arranged in a region adjacent to the rotation shaft 241. The pulley 213 and the pulley 214, which are driving part first jaw auxiliary pulleys, may be coupled to the rotation shaft 245. Here, the pulley 213 and the pulley 214 may be formed to be rotatable around the rotation shaft 245.

Here, the drawings illustrate that the driving part first jaw pulley is composed of two pulleys 211 and 212, the wire 301 is coupled to one pulley 211, and the wire 305 is coupled to the other pulley 212. However, an embodiment of the present disclosure is not limited thereto, and the driving part first jaw pulley may be composed of one pulley, and both the wire 301 and the wire 305 may be coupled to the one pulley.

As described above, the rotation shaft 241 is coupled to the first jaw driving motor (not shown) by the motor coupling part 251, and thus, when the first jaw driving motor (not shown) rotates for driving of the first jaw 101, the pulley 211 and the pulley 212, which are driving part first jaw pulleys, are rotated together with the rotation shaft 241, such that the wire 301 and the wire 305, which constitute the first jaw wire, are pulled or released.

The pulley 221 and the pulley 222, which are driving part second jaw rotation shafts, may be coupled to the rotation shaft 242 that is a driving part second jaw pulley. Here, the pulley 221 and the pulley 222 may be formed to rotate together with the rotation shaft 242.

In addition, the rotation shaft 246, which is a driving part second jaw auxiliary rotation shaft, may be arranged in a region adjacent to the rotation shaft 242. The pulley 223 and the pulley 224, which are driving part second jaw auxiliary pulleys, may be coupled to the rotation shaft 245. Here, the pulley 223 and the pulley 224 may be formed to be rotatable around the rotation shaft 246.

Here, the drawings illustrate that the driving part second jaw pulley is composed of two pulleys 221 and 222, the wire 302 is coupled to one pulley 221, and the wire 306 is coupled to the other pulley 222. However, an embodiment of the present disclosure is not limited thereto, and the driving part second jaw pulley may be composed of one pulley, and both the wire 302 and the wire 306 may be coupled to the one pulley.

As described above, the rotation shaft 242 is coupled to the second jaw driving motor (not shown) by the motor coupling part 252, and thus, when the second jaw driving motor (not shown) rotates for driving of the second jaw 102, the pulley 221 and the pulley 222, which are driving part second jaw pulleys, are rotated together with the rotation shaft 242, such that the wire 302 and the wire 306, which constitute the second jaw wire, are pulled or released.

The pulley 231, which is a driving part pitch pulley, may be coupled to the rotation shaft 243 that is a driving part pitch rotation shaft. Here, the pulley 231 may be formed to rotate together with the rotation shaft 243.

As described above, the rotation shaft 243 is coupled to a pitch driving motor (not shown) by the motor coupling part 253, and thus, when the pitch driving motor (not shown) rotates for a pitch motion, the wire 303 and the wire 304, which are pitch wires, are pulled or released as the pulley 231, which is a driving part pitch pulley, is rotated together with the rotation shaft 243.

Meanwhile, the pulley 215, the pulley 216, the pulley 217, the pulley 218, the pulley 225, the pulley 226, the pulley 227, and the pulley 228, which constitute the driving part relay pulley, may be formed to be rotatable around the rotation shaft 243 by inserting the rotation shaft 243 therethrough. Here, the pulley 215, the pulley 216, the pulley 217, and the pulley 218, which constitute the driving part first jaw relay pulley, may be arranged on one surface side of the pulley 231 that is a pitch pulley, and the pulley 225, the pulley 226, the pulley 227, and the pulley 228, which constitute the driving part second jaw relay pulley, may be arranged on the other surface side of the pulley 231.

In other words, the pulley 225 and the pulley 226, which are driving part second jaw first relay pulleys, the pulley 227 and the pulley 228, which are driving part second jaw second relay pulleys, the pulley 231, which is a driving part pitch pulley, and the pulley 217 and the pulley 218, which are driving part first jaw second relay pulleys, and the pulley 215 and the pulley 216, which are driving part first jaw first relay pulleys, are sequentially stacked and formed along the rotation shaft 243.

In addition, the pitch-yaw connector 232 may be coupled to the rotation shaft 243. The pitch-yaw connector 232 may be formed to rigidly connect the pulley 231, which is a driving part pitch pulley, to the pulley 219, the pulley 220, the pulley 229, and the pulley 230, which are driving part satellite pulleys, to allow the driving part satellite pulleys to be revolved around the rotation shaft 243 when the pulley 231 is rotated. This will be described in more detail below.

Here, the pitch-yaw connector 232 may be formed to rotate together with the rotation shaft 243. That is, the pulley 231 and the pitch-yaw connector 232 may be coupled to the rotation shaft 243, to be rotated together with the rotation shaft 243.

Here, the pitch-yaw connector 232 may be described as being formed in an approximately Y-shape as illustrated in FIG. 17, or may be described as being formed in a shape in which at least two extension parts 232a and 232b are formed to extend from the center thereof. In addition, a driving part first jaw satellite pulley central shaft 233 and a driving part second jaw satellite pulley central shaft 234 may be formed at ends of the extension parts 232a and 232b, respectively.

In addition, the pulley 219 and the pulley 220, which are driving part first jaw satellite pulleys, may be coupled to the driving part first jaw satellite pulley central shaft 233, and the pulley 229 and the pulley 230, which are driving part second jaw satellite pulleys, may be coupled to the driving part second jaw satellite pulley central shaft 234.

Accordingly, when the pulley 231, which is a driving part pitch pulley, is rotated together with the rotation shaft 243, the pulley 219, the pulley 220, the pulley 229, and the pulley 230, which are driving part satellite pulleys, are revolved around the rotation shaft 243. In other words, it may be said that the driving part first jaw satellite pulley central shaft 233 and the driving part second jaw satellite pulley central shaft 234 are rotated around the rotation shaft 243 while maintaining a constant distance from the rotation shaft 243 in a state in which the driving part first jaw satellite pulley central shaft 233 and the driving part second jaw satellite pulley central shaft 234 are spaced apart from the rotation shaft 243 by a certain extent.

That is, the driving part satellite pulley is formed to be movable relative to the driving part relay pulley and the rotation shaft 243 such that a relative position of the driving part satellite pulley with respect to the driving part relay pulley and the rotation shaft 243 may be changed. On the contrary the relative positions of the driving part pitch pulley and the driving part relay pulley remain constant.

In addition, when the pulley 231, which is a driving part pitch pulley, is rotated around the rotation shaft 243, the pulley 219, the pulley 220, the pulley 229, and the pulley 230, which are driving part satellite pulleys, are moved relative to the pulley 231, which is a driving part pitch pulley, such that the overall lengths of the wire 301, the wire 302, the wire 305, and the wire 306, which are jaw wires, in the driving part 200 are changed.

The wire 301, which constitutes the first jaw wire, is connected to the end tool 100 through the connection part 310 after being sequentially wound to come into contact with at least portions of the pulley 211, the pulley 213, the pulley 215, the pulley 219, and the pulley 217, in a state in which one end of the wire 301 is coupled to the pulley 211 by the first jaw wire-driving part coupling member (not shown).

In other words, the wire 301, which constitutes the first jaw wire, is connected to the end tool 100 through the connection part 310 after being sequentially passing through the driving part first jaw pulley 211, the driving part first jaw auxiliary pulley 213, the driving part first jaw first relay pulley 215, the driving part first jaw satellite pulley 219, and the driving part first jaw second relay pulley 217.

In other words, the wire 301, which constitute the first jaw wire, enters the driving part 200 after passing through the end tool 100 and the connection part 310, and then is fixedly coupled to the pulley 211, which is a driving part first jaw pulley, after being sequentially wound around the pulley 217, the pulley 219, the pulley 215, and the pulley 213.

Meanwhile, the wire 305, which constitute the first jaw wire, is connected to the end tool 100 through the connection part 310 after being sequentially wound to come into contact with at least portions of the pulley 212, the pulley 214, the pulley 216, the pulley 220, and the pulley 218, in a state in which one end of the wire 305 is coupled to the pulley 212 by the first jaw wire-driving part coupling member (not shown).

The wire 302, which constitutes the second jaw wire, is connected to the end tool 100 through the connection part 310 after being sequentially wound to come into contact with at least portions of the pulley 221, the pulley 223, the pulley 225, the pulley 229, and the pulley 227, in a state in which one end of the wire 302 is coupled to the pulley 221 by the second jaw wire-driving part coupling member (not shown).

Meanwhile, the wire 306, which constitutes the second jaw wire, is connected to the end tool 100 through the connection part 310 after being sequentially wound to come into contact with at least portions of the pulley 222, the pulley 224, the pulley 226, the pulley 230, and the pulley 228, in a state in which one end of the wire 306 is coupled to the pulley 222 by the second jaw wire-driving part coupling member (not shown).

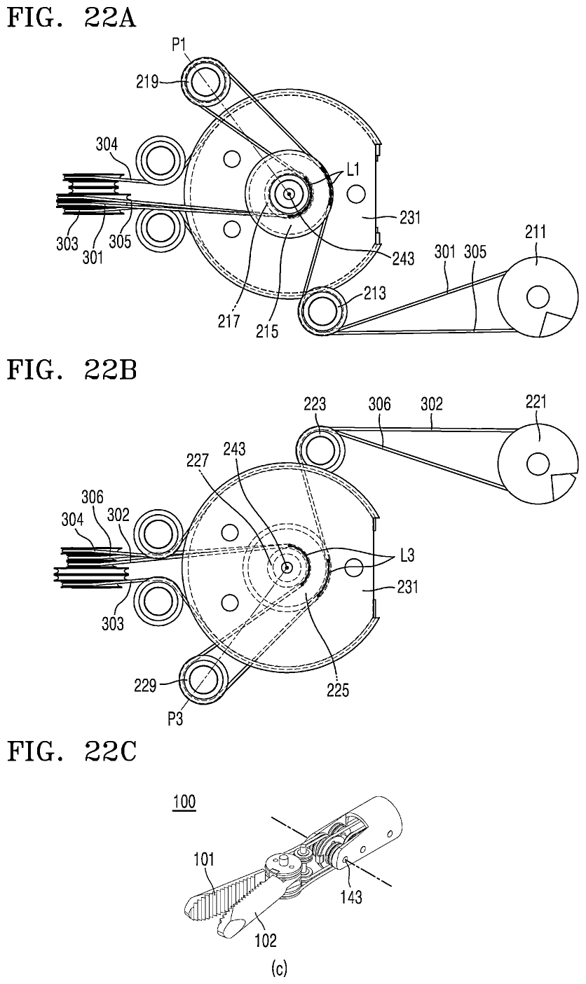

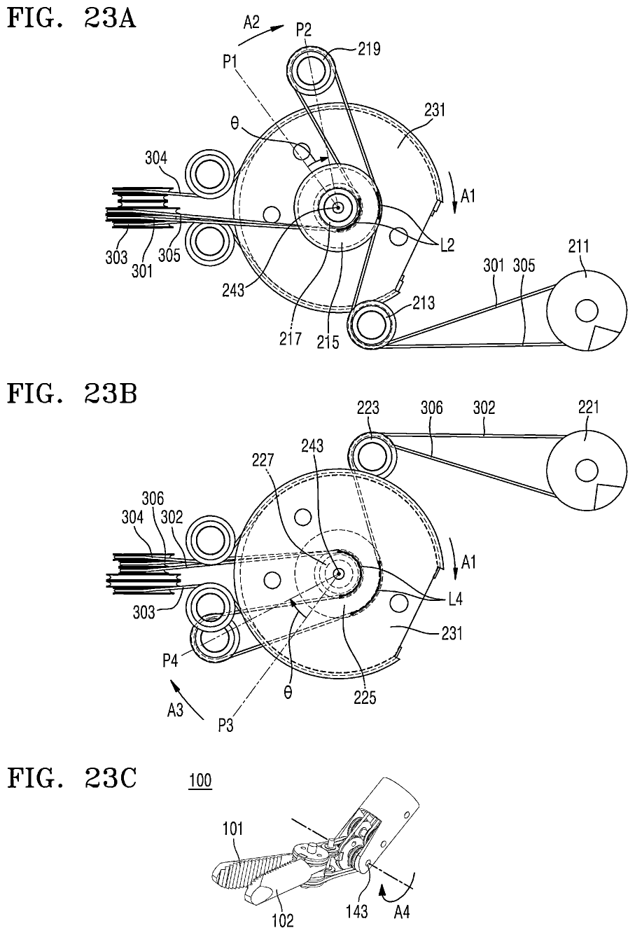

FIGS. 22 and 23 are diagrams illustrating a pitch motion of the instrument illustrated in FIG. 11. Here, for convenience of description, only pulleys and wires associated with rotation of the first jaw are illustrated in (a) of FIG. 22 and (b) of FIG. 23, and only pulleys and wires associated with rotation of the second jaw are illustrated in (b) of FIG. 22 and (b) of FIG. 23. In addition, (c) of FIG. 22 and (c) of FIG. 23 illustrate a pitch motion of the end tool according to a pitch motion of the driving part.

Here, in the instrument 30 according to an embodiment of the present disclosure, when the driving part satellite pulley is moved relative to the driving part relay pulley, the overall length of the jaw wire within the driving part 200 is changed, and thus, a pitch motion of the end tool 100 is performed. In particular, in the instrument 30 according to an embodiment of the present disclosure, when the driving part pitch pulley is rotated, the driving part satellite pulley is revolved around the (common) rotation shaft of the driving part relay pulley and the driving part pitch pulley such that a path length of the jaw wire wound around the driving part relay pulley is changed, and thus, a pitch motion of the end tool is performed.

In detail, when motion compensation for the pitch motion is not separately performed in the driving part, the pitch motion itself cannot be performed in the end tool.

Meanwhile, in order for the end tool to perform a pitch motion, the wire 301 and the wire 305 need to be further wound around the pulley 113 by ΔSpitch and the wire 302 and the wire 306 need to be further unwound from the pulley 114 by ΔSpitch. However, when such compensation is not performed in the driving part, the pitch motion itself cannot be performed in the end tool.

In order to perform motion compensation for the pitch motion as described above, in the instrument 30 according to an embodiment of the present disclosure, the driving part pitch pulleys are rotated while the driving part satellite pulley is revolved, such that the jaw wires are wound around or released from the driving part relay pulley, which allows the movement of the jaw wires to be compensated for by the rotation of the driving part pitch pulley.

In other words, when the pulley 231, which is a driving part pitch pulley, is rotated together with the rotation shaft 243, the driving part satellite pulleys are revolved around the rotation shaft 243. In addition, as the driving part satellite pulleys are revolved around the rotation shaft 243, the length by which the jaw wire is wound around the driving part relay pulley is changed. That is, the jaw wire wound on the side of the end tool 100 due to rotation of the pulley 231 is released by the same amount on the side of the driving part 200, and the jaw wire unwound on the side of the end tool 100 is wound by the same amount on the side of the driving part 200, such that the pitch motion does not affect the yaw motion.

In other words, when the end tool performs the pitch motion by the rotation of the driving part pitch pulley, the jaw wire (responsible for a yaw motion and an actuation motion) is also moved by the pitch motion. That is, as the pitch rotation is performed around the rotation shaft 143 of the end tool 100, both strands of the jaw wire coupled to one jaw are pulled, and both strands thereof coupled to the other jaw are released. Accordingly, it may be described that, in the present disclosure, in order to compensate for the movement of the jaw wire, when the pitch motion of the end tool is performed, the overall length of the jaw wire within the driving part is changed while the driving part satellite pulley is moved relative to the driving part relay pulley, such that the jaw wire is released (or pulled) on the side of the end tool as much as the jaw wire is pulled (or released) on the side of the driving part, thereby compensating for the movement of the jaw wire when the pitch motion of the end tool is performed.

Hereinafter, the pitch motion will be described in more detail.

When the pulley 231, which is a driving part pitch pulley, is rotated in the direction of an arrow A1 (i.e., in the clockwise direction in the drawing) in order to perform the pitch motion, the pitch-yaw connector (see 232 of FIG. 15) is rotated in the direction of the arrow A1 together with the pulley 231, and accordingly, the pulley 219 and the pulley 220, which are driving part satellite pulleys fixedly coupled to the pitch-yaw connector (see 232 of FIG. 15), are revolved as a whole in the direction of an arrow A2 of (a) of FIG. 23 (i.e., in the clockwise direction in the drawing) around the rotation shaft 243 by θ. That is, when the pulley 231 is rotated, the pulley 219 and the pulley 220 are rotated by θ from the position of P1 of (a) of FIG. 22 to the position of P2 of (a) of FIG. 23. In other words, it may be described that, when the driving part pitch pulley is rotated, the driving part satellite pulley is moved in conjunction with the driving part pitch pulley.

At the same time, when the pulley 231, which is a driving part pitch pulley, is rotated in the direction of the arrow A1 (i.e., in the clockwise direction in the drawing), the pitch-yaw connector (see 232 of FIG. 8) is rotated in the direction of the arrow A1 together with the pulley 231, and accordingly, the pulley 229 and the pulley 230, which are driving part satellite pulleys fixedly coupled to the pitch-yaw connector (see 232 of FIG. 8), are revolved as a whole in the direction of an arrow A3 of (b) of FIG. 23 (i.e., in the clockwise direction in the drawing) around the rotation shaft 243 by θ. That is, when the pulley 231 is rotated, the pulley 229 and the pulley 230 are rotated by θ from the position of P3 of (b) of FIG. 22 to the position of P4 of (b) of FIG. 23. In other words, it may be described that, when the driving part pitch pulley is rotated, the driving part satellite pulley is moved in conjunction with the driving part pitch pulley.

Meanwhile, at this time, the positions of the pulley 215, the pulley 216, the pulley 217, the pulley 218, the pulley 225, the pulley 226, the pulley 227, and the pulley 228, which are driving part relay pulleys coupled to the rotation shaft 243, are not changed. That is, the relative positions of the pulley 211, which is a driving part jaw pulley, the pulley 231, which is a driving part pitch pulley, and the pulley 215, the pulley 216, the pulley 217, and the pulley 218, which are driving part relay pulleys, remain constant. Similarly, the relative positions of the pulley 221, which is a driving part jaw pulley, the pulley 231, which is a driving part pitch pulley, and the pulley 225, the pulley 226, the pulley 227, and the pulley 228, which are driving part relay pulleys, remain constant.

In addition, as described above, the relative position of the driving part satellite pulley with respect to the driving part relay pulley is changed as the driving part satellite pulley is revolved, and accordingly, the length of each wire wound around the driving part relay pulley, that is, the path length, is changed. Here, the driving part relay pulley includes the pulley 215, which is a driving part first jaw first relay pulley, and the pulley 217, which is a driving part first jaw second relay pulley, and thus, the path length means the sum of the length by which the wire 301 is wound around the pulley 215, and the length by which the wire 301 is wound around the pulley 217 (or, the sum of the length by which the wire 305 is wound around the pulley 216, and the length by which the wire 305 is wound on the pulley 218).

That is, as compared to a path length L1 by which the wire 301 and the wire 305, which constitute the first jaw wire, are wound around the driving part relay pulleys at the position in (a) of FIG. 22, a path length L2 by which the first jaw wire is wound around the driving part relay pulleys at the position in (a) of FIG. 23 is reduced, and the first jaw wire is further released on the side of the driving part 200 as much as the path length is reduced (L1−L2). That is, the overall lengths of the wire 301 and the wire 305, which constitute the first jaw wire, within the driving part 200 are reduced. In addition, as the overall length of the first jaw wire within the driving part 200 is reduced, the overall length of the first jaw wire within the end tool 100 is increased as much as the first jaw wire is unwound.

In contrast, when the pulley 231, which is a driving part pitch pulley, is rotated in the direction of the arrow A1, as compared to a path length L3 by which the wire 302 and the wire 306, which constitute the second jaw wire, are wound around the driving part relay pulleys at the position in (b) of FIG. 23, a path length L4 by which the second jaw wire is wound around the driving part relay pulleys at the position in (b) of FIG. 23 is increased, and the second jaw wire is further pulled on the side of the driving part 200 as much as the path length is increased (L4−L3). That is, the overall lengths of the wire 302 and the wire 306, which constitute the second jaw wire, within the driving part 200 are increased. In addition, as the overall length of the second jaw wire within the driving part 200 is increased, the overall length of the second jaw wire within the end tool 100 is reduced as much as the second jaw wire is pulled.

As such, when the pulley 231, which is a driving part pitch pulley, is rotated in the direction of the arrow A1 for a pitch motion, the relative position of the driving part satellite pulley is changed as the driving part satellite pulley is moved relative to the driving part pitch pulley and the driving part relay pulley. In addition, due to the relative movement of the driving part satellite pulley, the overall length of the first jaw wire within the driving part 200 is reduced, and the overall length of the first jaw wire within the end tool 100 is increased. At the same time, due to the relative movement of the driving part satellite pulley, the overall length of the second jaw wire within the driving part 200 is increased, and the overall length of the second jaw wire within the end tool 100 is reduced.

Accordingly, when viewed from the side of the end tool 100, when the pulley 231, which is a driving part pitch pulley, is rotated in the direction of the arrow A1, the wire 301 and the wire 305, which are two strands of the first jaw wire, are released and the wire 302 and the wire 306, which are two strands of the second jaw wire, are pulled, such that the end tool 100 performs a pitch motion in the direction of an arrow A4 around the rotation shaft 143.

Here, the path length may be defined as a length of the jaw wire from a point at which the jaw wire enters the driving part first relay pulley, to a point at which the jaw wire exits from the driving part second relay pulley through the driving part satellite pulley. That is, the path length may be defined as a length of the wire 301, which is a jaw wire, from a point at which the jaw wire enters the pulley 215, which is a driving part first relay pulley, to a point at which the jaw wire exits from the pulley 217, which is a driving part second relay pulley, through the pulley 219 that is a driving part satellite pulley.

In other words, the path length may be defined as the length of the jaw wire from an initial contact point of the jaw wire with the driving part relay pulley to a final contact point of the jaw wire with the driving part relay pulley on the arrangement path of the jaw wire that connects the end tool jaw pulley to the driving part jaw pulley. That is, the path length may be defined as the length of the jaw wire from an initial contact point of the wire 301, which is a jaw wire, with the pulley 215, which is a driving part first relay pulley, to a final contact point of the wire 301 with the pulley 217, which is a driving part second relay pulley.

Meanwhile, as the above-described path length is changed as the driving part satellite pulley is moved relative to the driving part relay pulley, the overall length of the jaw wire within the driving part 200 is also changed. In addition, as the overall length of the jaw wire within the driving part 200 is changed, the overall length of the jaw wire within the end tool 100 is also changed. However, it may be said that, because the overall length of the jaw wire within the end tool 100 is also increased (or reduced) as much as the overall length of the jaw wire within the driving part 200 is increased (reduced), the overall length of the jaw wire is not changed (assuming that elastic deformation or the like is not considered).

Accordingly, when the driving part pitch pulley is rotated, the wire 301/wire 305, which constitute the first jaw wire, are released on the side of the driving part 200 as much as the wire 301/wire 305, which constitute the first jaw wire, are pulled on the side of the end tool 100, and as a result, a pitch motion is enabled.

Meanwhile, as described above, the end tool 100 of the instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304, which are pitch wires.

Thus, when the pulley 231, which is a driving part pitch pulley, is rotated in the direction of the arrow A1, the wire 304 is wound around the pulley 231 and the wire 303 is released from the pulley 231, due to the rotation of the pulley 231. Accordingly, as the pulley 131, which is an end tool pitch pulley connected to the opposite side of the wire 303 and the wire 304, is rotated in the direction of the arrow A2 around the rotation shaft 143, the pitch motion may be more surely and reliably performed.

Here, among the pulleys that are rotated around the rotation shaft 143, which is an end tool pitch rotation shaft, the pulley 131, which is an end tool pitch pulley in contact with the wire 303 and the wire 304, which are pitch wires, may be formed to have a diameter different from those of the pulley 113, the pulley 114, the pulley 123, and the pulley 124, which are end tool jaw pitch main pulleys in contact with the wire 301, the wire 305, the wire 302, and the wire 306, which are jaw wires.

In this case, when the rotation shaft 143 is rotated, the lengths by which the respective wires are wound around or unwound from the respective pulleys are different from each other. For example, when the diameter of the end tool pitch pulley is 6φ, the diameter of the end tool jaw pitch main pulley is 4φ, and the rotation shaft 143 is rotated by 90°, the length by which the pitch wire is wound around the end tool pitch pulley may be 1.5π, whereas the length by which the jaw wire is wound around the end tool jaw pitch main pulley may be 1π.

From this perspective, the ‘length’ by which the wire is wound around or unwound from the pulley may be defined as a ‘rotation amount’. The rotation amount is a concept different from a rotation angle, and may be calculated as (diameter*rotation angle/360°*π).

In this case, because basically the pulley 231, which is a driving part pitch pulley, is directly connected to the pulley 131, which is an end tool pitch pulley, by the wire 303 and the wire 304, which are pitch wires, the rotation amount of the driving part pitch pulley is equal to that of the end tool pitch pulley. That is, the pitch wire is released from or wound around the end tool pitch pulley as much as the pitch wire is wound around or released from the driving part pitch pulley.

Meanwhile, the relationship of (diameter of end tool pitch pulley:diameter of end tool jaw pitch main pulley)=(rotation amount of wire wound around end tool pitch pulley: rotation amount of wire wound around end tool jaw pitch main pulley) may be satisfied.

As described above, when, in the end tool 100, the length by the pitch wire is wound around the end tool pitch pulley is different from the length by which the jaw wire is wound around the end tool jaw pitch main pulley, in the driving part 200, the length by which the pitch wire is to be released needs to be different from the length by which the jaw wire is to be released, by the same proportion.

To this end, the relationship of (diameter of end tool pitch pulley:diameter of end tool jaw pitch main pulley)=(diameter of driving part pitch pulley:diameter of driving part relay pulley) may be satisfied.

For example, when the ratio of (diameter of end tool pitch pulley:diameter of end tool jaw pitch main pulley) is 6:4, the ratio of (diameter of driving part pitch pulley:diameter of driving part relay pulley) may also be 6:4. According to this ratio, the diameter of the driving part pitch pulley may be 9φ, and the diameter of the driving part relay pulley may be 6φ.

However, here, the driving part relay pulley may include two (or more) pulleys including the driving part first relay pulley and the driving part second relay pulley. In addition, the sum of the diameters of the driving part first relay pulley and the driving part second relay pulley may be defined as the diameter of the driving part relay pulley.

For example, when the diameter of the driving part relay pulley is 6φ, there are several possible combinations for (diameter of driving part first relay pulley, diameter of driving part second relay pulley), including (1φ, 5φ), (2φ, 4φ), (3φ, 3φ), (4φ, 2φ), and (5φ, 1φ). Here, the drawings illustrate that the diameter of the pulley 215, which is a driving part first relay pulley, is 4φ, and the diameter of the pulley 217, which is the driving part second relay pulley, is 2φ.

In addition, it may be described that (rotation amount of driving part first relay pulley+rotation amount of driving part second relay pulley) is proportional to the rotation amount of the driving part pitch pulley.

However, although the ratio of (diameter of end tool pitch pulley:diameter of end tool jaw pitch main pulley) is not exactly equal to the ratio of (diameter of driving part pitch pulley:diameter of driving part relay pulley), when the diameters of the pulleys are selected to make these ratios similar to each other, an objective of the present disclosure, which is to compensate for the movement of the jaw wire according to rotation of the driving part pitch pulley, may be achieved to some extent.

The final pitch motion process will be described again as follows.

Hereinafter, an example will be described in which the diameter of the end tool pitch pulley is 6φ, the diameter of the end tool jaw pitch main pulley is 4φ, the diameter of the driving part pitch pulley is 9φ, and the diameter of the driving part relay pulley is 6φ.

First, for a pitch motion, the pulley 231, which is a driving part pitch pulley of the driving part 200, is rotated by 60° to wind the wire 304, which is a pitch wire, while releasing the wire 303. At this time, the lengths by which the wire 303/wire 304 are respectively wound and unwound is 1.5.

Accordingly, as the wire 304 is pulled by 1.5π and the wire 303 is released by 1.5π in the end tool 100, the pulley 131, which is an end tool pitch pulley, is rotated by 90° corresponding to 1.5π.

Meanwhile, when the pulley 131 is pitch-rotated around the rotation shaft 143, the jaws 101 and 102 and the pulley 111/pulley 112 are also pitch-rotated around the rotation shaft 143. Accordingly, both the wire 301 and the wire 305, which constitute the first jaw wire coupled to the pulley 111, are pulled, and both the wire 302 and the wire 306, which constitute the second jaw wire coupled to the pulley 121, are released. At this time, the angles by which the end tool pitch pulley and the end tool jaw pitch main pulley are rotated are equal to each other, i.e., 90°, and thus, the lengths by which the jaw wires are wound around or released from the end tool jaw pitch main pulley are 1π.

Meanwhile, because the pulley 231 and the pulley 219/pulley 220 are rigidly connected to each other by the pitch-yaw connector 232, when the pulley 231 is rotated by 60° around the rotation shaft 243, the pulley 219/pulley 220 are revolved by 60° around the rotation shaft 243.

In addition, as described above, as the pulley 219/pulley 220 are revolved, the jaw wires are wound around or released from the pulley 215 and the pulley 216 of which the sum of the diameters is 6φ, by 1π corresponding to a revolution angle of 60°. That is, the wire 301 and the wire 305, which constitute the first jaw wire, are released as a whole, and the wire 302 and the wire 306, which constitute the second jaw wire, are pulled as a whole.

In other words, the overall path lengths of the wire 301 and the wire 305 wound around the pulley 215, the pulley 216, the pulley 217, and the pulley 218, which are driving part first jaw relay pulleys, are reduced, and the wire 301 and the wire 305 are released as much as the path lengths are reduced. In addition, the overall path lengths of the wire 302 and the wire 306 wound around the pulley 225, the pulley 226, the pulley 227, and the pulley 228, which are driving part second jaw relay pulleys, are increased, and the wire 302 and the wire 306 are pulled as much as the path lengths are increased.

That is, the wire 301 and the wire 305, which constitute the first jaw wire, are released on the side of the driving part 200 as much as the wire 301 and the wire 305 are pulled on the side of the end tool 100, and thus, the movement of the jaw wire due to the pitch motion is compensated for. Similarly, the wire 302 and the wire 306, which constitute the second jaw wire, are released on the side of the driving part 200 as much as the wire 302 and the wire 306 are pulled on the side of the end tool 100, and thus, the movement of the jaw wire due to the pitch motion is compensated for.

Accordingly, by releasing (or pulling) the jaw wires on the side of the driving part 200 by a length equal to the length by which the jaw wires are wound around (or released from) on the side of the end tool 100 according to the pitch motion, the pitch motion may be performed independently without affecting rotation of the jaw around the yaw shaft.

That is, when the driving part pitch pulley and the driving part satellite pulley are rigidly connected to each other, and the driving part pitch pulley is rotated around the rotation shaft 243, the path length of the jaw wire wound around the driving part relay pulley is changed as the driving part satellite pulley is revolved around the rotation shaft 243. In addition, the change in the path length of the jaw wire compensates for the movement of the jaw wires on the side of the end tool due to the pitch motion, and as a result, the pitch motion may be independently performed.

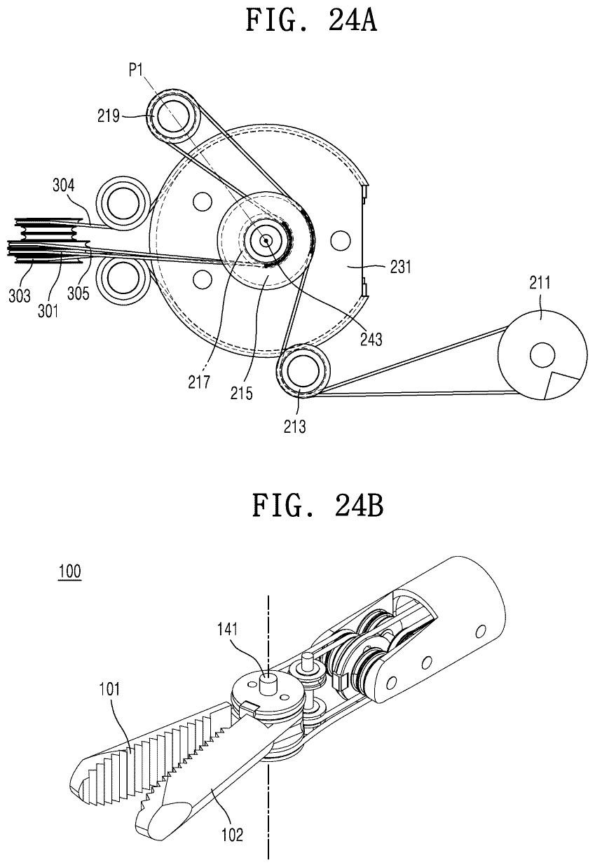

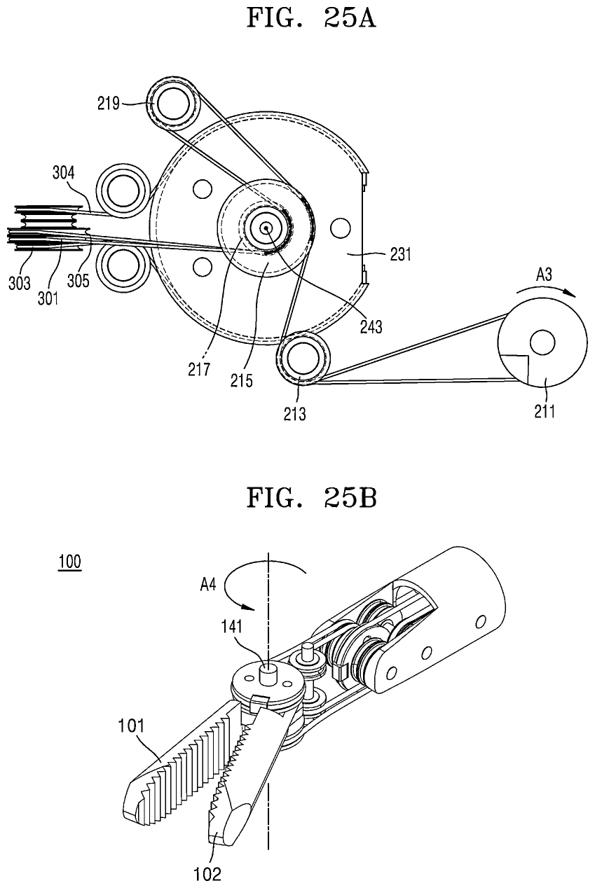

FIGS. 24 and 25 are diagrams illustrating a yaw motion of the instrument illustrated in FIG. 11.

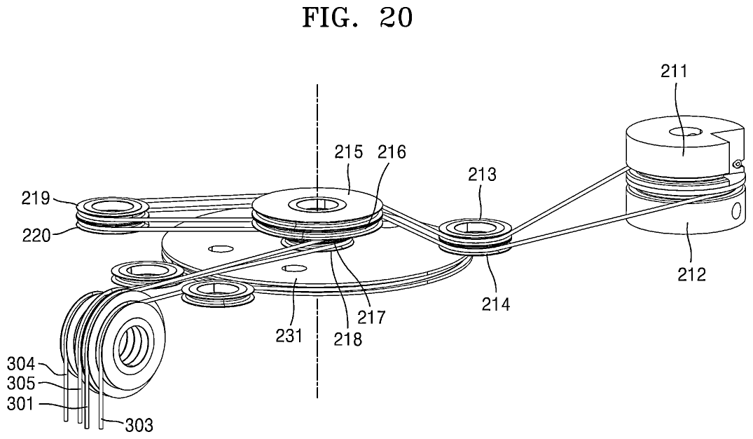

Referring to FIGS. 20, 21, 24, and 25 and the like, when the pulley 211, which is a driving part first jaw pulley, is rotated in the direction of the arrow A3 for a yaw motion, any one of the wire 301 and the wire 305, which constitute the first jaw wire, is wound around the pulley 211 and the other one is released from the pulley 211 in response to the rotation of the pulley 211. Accordingly, as the pulley 111, which is an end tool first jaw pulley connected to the opposite side of the wire 301 and the wire 305, is rotated in the direction of the arrow A4, the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wire 301 and the wire 305 are wound around or released from the driving part satellite pulley and the driving part relay pulley occurs.

Accordingly, the driving part pitch pulley rigidly connected to the driving part satellite pulley is not rotated, and the wire 303 and the wire 304, which are pitch wires, are not wound or released and maintain their positions.

Similarly, when the pulley 221, which is a driving part second jaw pulley, is rotated for a yaw motion, any one of the wire 302 and the wire 306, which constitute the second jaw wire, is wound around the pulley 221 and the other one is released from the pulley 221, in response to the rotation of the pulley 221. Accordingly, as the pulley 121, which is an end tool second jaw pulley connected to the opposite side of the wire 302 and the wire 306, is rotated in any one direction, the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wire 302 and the wire 306 are wound around or released from the driving part satellite pulley and the driving part relay pulley occurs.

Accordingly, the driving part pitch pulley rigidly connected to the driving part satellite pulley is not rotated, and the wire 303 and the wire 304, which are pitch wires, are not wound or released and maintain their positions.

Accordingly, even when the pulley 211 or the pulley 221, which is a driving part jaw pulley, is rotated for a yaw motion or an actuation motion, the overall lengths of the wire 301, the wire 302, the wire 305, and the wire 306, which are jaw wires, within the driving part 200 remain constant.

As described above, in the instrument 30 according to an embodiment of the present disclosure, when the driving part pitch pulley is rotated, the driving part satellite pulley is revolved around the rotation shaft of the driving part pitch pulley to change the path length of the jaw wire wound around the driving part relay pulley, and thus, the jaw wire is wound or released in response to the rotation of the driving part pitch pulley, such that the movement of the jaw wire due to the pitch drive may be offset or compensated for, and as a result, the effect of separating the pitch motion and the yaw motion from each other may be obtained.

However, as described above, the pitch motion and the yaw motion are not limited to being mechanically separated from each other, and may be independently separated from each other by a processor of the present disclosure according to an embodiment, and thus able to perform each of the pitch motion and the yaw motion.

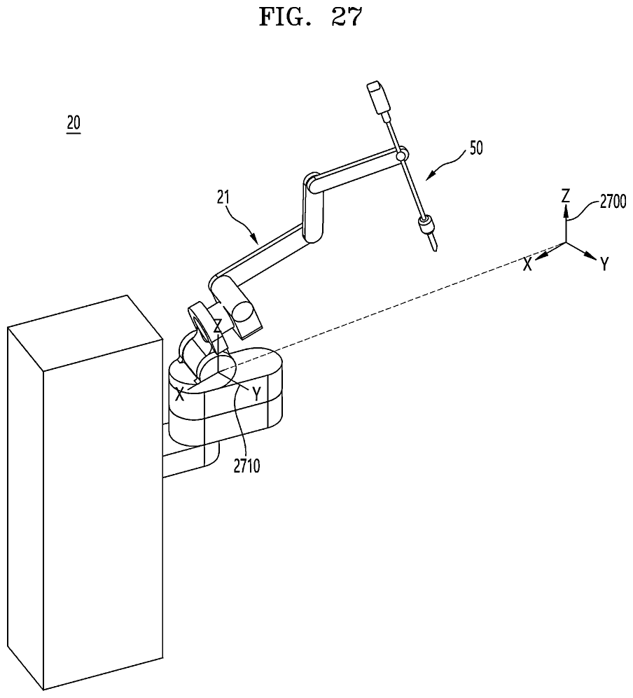

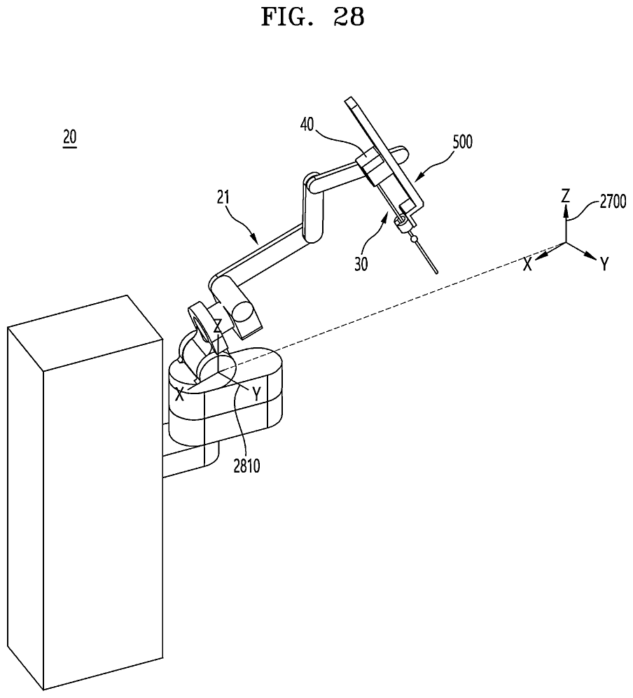

FIG. 26 is a flowchart for describing an example of a method of generating coordinate system transformation information in a robotic surgical system, according to an embodiment. FIGS. 27 and 28 are perspective views for describing an example of a coordinate system used in a robotic surgical system, according to an embodiment.

Referring to FIG. 26, the method of generating coordinate system transformation information in a robotic surgical system consists of operations processed in time series by the device 1000 or the processor 1010 illustrated in FIG. 4. Thus, the descriptions provided above regarding the device 1000 or the processor 1010 illustrated in FIG. 4, which are even omitted below, may also be applied to the method of generating coordinate system transformation information in a robotic surgical system shown in FIG. 26.

In addition, as described above with reference to FIG. 4, at least one of the operations of the method of generating coordinate system transformation information in a robotic surgical system shown in FIG. 26 may be processed by the master robot 10, a camera robot, or a surgical robot.

First, in operation S2610, the processor 1010 may calculate a first angle difference between a preset reference coordinate system and a first robot base coordinate system. Here, the first angle difference may be calculated based on a dial manipulation value of a user obtained from a first angle measurement dial mounted on a first robot.

Thereafter, in operation S2620, the processor 1010 may calculate a second angle difference between the preset reference coordinate system and a second robot base coordinate system. Here, the second angle difference may be calculated based on a dial manipulation value of the user obtained from a second angle measurement dial mounted on a second robot.

In operations S2610 and S2620, each of the first robot and the second robot may be any one of a camera robot equipped with a laparoscopic surgical camera or a surgical robot equipped with a surgical instrument. However, for convenience of description, hereinafter, it is assumed that the first robot is a camera robot and the second robot is a surgical robot.

Referring to FIG. 27, the robotic arm unit 21 included in a camera robot 2700 may use a camera robot base coordinate system 2720. That is, the movement of the robotic arm unit 21 included in the camera robot 2700 may be expressed based on the camera robot base coordinate system 2720.

Here, the robot base coordinate system may be defined as a fixed coordinate system for defining the movement and position of a robot. In other words, the robot base coordinate system may be determined based on a stationary position and direction of the robot (the origin or reference point of the coordinate system). For example, as described above with reference to FIG. 9, the body and the passive arm unit of the camera robot 2700 do not change in position or direction unless an external force acts after their arrangement is completed, and thus, the camera robot base coordinate system 2720 may be determined based on the position of the body or the passive arm unit. For example, the camera robot base coordinate system 2720 may be determined by using a connection point between the active arm unit and the passive arm unit included in the camera robot 2700, as a reference point (or an origin). As another example, the camera robot base coordinate system 2720 may be determined by using a connection point between the body and the active arm unit included in the camera robot 2700, as a reference point (or an origin).

Meanwhile, the laparoscopic surgical camera 50 included in the camera robot 2700 may use a separate laparoscopic surgical camera coordinate system (not shown). According to an embodiment, the processor 1010 may generate coordinate system transformation information between the camera robot base coordinate system 2720 and the laparoscopic surgical camera coordinate system (not shown), based on mechanical information of the camera robot 2700. The processor 1010 may express a movement of the laparoscopic surgical camera 50 by using the camera robot base coordinate system 2720, based on the coordinate system transformation information between the camera robot base coordinate system 2720 and the laparoscopic surgical camera coordinate system (not shown).

According to an embodiment, an angle measurement dial (not shown) may be mounted on the camera robot 2700. For example, the angle measurement dial mounted on the camera robot 2700 may use the camera robot base coordinate system 2720. That is, the angle measurement dial mounted on the camera robot 2700 may be installed at a connection point between the active arm unit and the passive arm unit included in the camera robot 2700, or at a connection point between the body and the active arm unit included in the camera robot 2700.

According to an embodiment, the angle measurement dial (not shown) mounted on the camera robot may obtain a dial manipulation value of the user by receiving physical dial manipulation (e.g., rotation manipulation) from the user who wants to measure an angle difference between a preset reference coordinate system 2710 and the camera robot base coordinate system 2720. In addition, the processor 1010 may calculate an angle difference between the reference coordinate system 2710 and the camera robot base coordinate system 2720, based on the dial manipulation value of the user obtained from the angle measurement dial (not shown) mounted on the camera robot 2700.

Here, the reference coordinate system may be defined as a coordinate system that serves as a reference for generating coordinate system transformation information between the camera robot base coordinate system 2720 and a surgical robot base coordinate system to be described below. For example, the reference coordinate system may be set in advance by using the position of an object that does not move and has a fixed position in an operating room as a reference point (or an origin), considering the characteristics of the operating room where the robotic surgical system is arranged. For example, the reference coordinate system may be set in advance to be a coordinate system used by a surgical bed, by using the surgical bed in the operating room where the camera robot (or the surgical robot) is arranged, as a reference point. As another example, the reference coordinate system may be set in advance to be a coordinate system used by the master robot 10, by using the master robot 10 included in the robotic surgical system, as a reference point. As another example, the reference coordinate system may be set in advance to be a camera robot base coordinate system used by the camera robot, by using the camera robot as a reference point.

Meanwhile, according to an embodiment, the angle measurement dial (not shown) mounted on the camera robot may obtain only a difference in yaw value between the preset reference coordinate system 2710 and the camera robot base coordinate system 2720, excluding a difference in roll value and a difference in pitch value. In other words, the first angle difference calculated by the processor 1010 based on the dial manipulation value of the user obtained from the angle measurement dial (not shown) mounted on the camera robot 2700 may refer to a difference in yaw value of the reference point of the camera robot base coordinate system with respect to the reference coordinate system.

Because the camera robot 2700 and each reference point of the reference coordinate system (e.g., the surgical bed or the master robot) are all arranged on a flat floor of the operating room, each axis of the camera robot base coordinate system and the reference coordinate system (e.g., the x-axis, the y-axis, or the z-axis) is parallel or perpendicular to the ground (e.g., the floor of the operating room), and thus, there may be no difference in roll value or pitch value between the preset reference coordinate system 2710 and the camera robot base coordinate system 2720. Accordingly, the user may measure only a difference in yaw value through manipulation of the first angle measurement dial. Here, the difference in yaw value may refer to a difference in azimuth of the camera robot base coordinate system (or the reference point of the camera robot base coordinate system) with respect to an axis of the reference coordinate system in a direction perpendicular to the ground, based on the reference coordinate system. In other words, the processor 1010 may calculate an azimuth OcRobot of the camera robot base coordinate system 2720 with respect to the preset reference coordinate system 2710, based on a dial manipulation value of the user obtained from the angle measurement dial mounted on the camera robot 2700.

Referring to FIG. 28, the robotic arm unit 21 included in a surgical robot 2800 may use a surgical robot base coordinate system 2820. Meanwhile, the definitions of the base coordinate system and the reference coordinate system described above with reference to FIG. 27 may be applied equally to FIG. 28, and thus, redundant descriptions will be omitted.

According to an embodiment, the surgical robot base coordinate system 2820 may be determined based on the position of the body or the passive arm unit included in the surgical robot 2800. For example, the surgical robot base coordinate system 2820 may be determined by using a connection point between the active arm unit and the passive arm unit included in the surgical robot 2800, as a reference point. As another example, the surgical robot base coordinate system 2820 may be determined by using a connection point between the body and the active arm unit included in the surgical robot 2800, as a reference point.

According to an embodiment, the preset reference coordinate system may be any one of a coordinate system used by a surgical bed in an operating room where the surgical robot is arranged, or a coordinate system used by the master robot 10 included in the robotic surgical system. According to another embodiment, the reference coordinate system 2810 may be a camera robot base coordinate system used by the camera robot.

According to an embodiment, an angle measurement dial (not shown) mounted on the surgical robot 2800 may use the surgical robot base coordinate system 2820. For example, the angle measurement dial mounted on the surgical robot 2800 may be installed at a connection point between the active arm unit and the passive arm unit included in the surgical robot 2800, or at a connection point between the body and the active arm unit included in the surgical robot 2800.

According to an embodiment, the angle measurement dial (not shown) mounted on the surgical robot 2800 may obtain a dial manipulation value of a user by receiving physical dial manipulation (e.g., rotation manipulation) from the user who wants to measure an angle difference between the preset reference coordinate system 2810 and the surgical robot base coordinate system 2820. In addition, the processor 1010 may calculate an angle difference between the reference coordinate system 2810 and the surgical robot base coordinate system 2820, based on the dial manipulation value of the user obtained from the angle measurement dial (not shown) mounted on the surgical robot.

According to another embodiment, the angle measurement dial (not shown) mounted on the surgical robot 2800 may obtain only a difference in yaw value between the preset reference coordinate system 2810 and the surgical robot base coordinate system 2820, excluding a difference in roll value and a difference in pitch value. In other words, the second angle difference calculated by the processor 1010 based on the dial manipulation value of the user obtained from the angle measurement dial (not shown) mounted on the surgical robot 2800 may refer to a difference in yaw value of the reference point of the surgical robot base coordinate system with respect to the reference coordinate system. Meanwhile, the difference in yaw value may refer to a difference in azimuth of the surgical robot base coordinate system (or the reference point of the surgical robot base coordinate system) with respect to an axis of the reference coordinate system in a direction perpendicular to the ground, based on the reference coordinate system. For example, the processor 1010 may calculate an azimuth θ_{sRobot} of the surgical robot base coordinate system 2820 with respect to the preset reference coordinate system 2810, based on a dial manipulation value of the user obtained from the angle measurement dial (not shown) mounted on the surgical robot 2800.

Referring again to FIG. 26, in operation S2630, the processor 1010 may generate coordinate system transformation information between the first robot base coordinate system and the second robot base coordinate system, based on the first angle difference and the second angle difference.

For example, the processor 1010 may calculate a relative angle between the first robot base coordinate system and the second robot base coordinate system, based on the first angle difference and the second angle difference.

According to an embodiment, the processor 1010 may calculate a relative azimuth between the camera robot base coordinate system and the surgical robot base coordinate system. For example, the processor 1010 may calculate the relative azimuth based on an azimuth θ_{cRobot} of the camera robot base coordinate system 2720 with respect to the reference coordinate system, and an azimuth θ_{sRobot} of the surgical robot base coordinate system 2820 with respect to the reference coordinate system. For example, the processor 1010 may calculate the relative azimuth as θ_{cRobot}−θ_{sRobot}, which is a difference between the two azimuths. According to another embodiment, the processor 1010 may calculate a relative azimuth between a first surgical robot base coordinate system and a second surgical robot base coordinate system, as θ_{sRobot1}−θ_{sRobot2}, based on an azimuth θ_{sRobot} of the first surgical robot base coordinate system with respect to the reference coordinate system, and an azimuth θ_{sRobot2} of the second surgical robot base coordinate system with respect to the reference coordinate system.

Meanwhile, the coordinate system transformation information may include information about a rotation transformation matrix. Here, the rotation transformation matrix may refer to a matrix for transforming a movement in any one three-dimensional coordinate system (e.g., a rotational movement) into a movement in another three-dimensional coordinate system. For example, the rotation transformation matrix may be calculated by using information about the axis and angle of rotation. For example, the processor 1010 may calculate a relative angle (e.g., a roll angle θᵣ, a pitch angle θₚ, or a yaw angle θ_{y})) between the first robot base coordinate system (e.g., a camera robot base coordinate system) and the second robot base coordinate system (e.g., a surgical robot base coordinate system) by using the first angle difference and the second angle difference, and calculate, based on Equation 1 below, a rotation transformation matrix between the camera robot base coordinate system and the surgical robot base coordinate system based on the calculated relative angle.

$${R}_{z} ( {θ}_{r} ) {R}_{y} ( {θ}_{p} ) {R}_{x} ( {θ}_{y} ) = \qquad [ Equation 1 ]$$

$$[ \begin{matrix}cos {θ}_{r} cos {θ}_{p} & cos {θ}_{r} sin {θ}_{p} sin {θ}_{y} - sin {θ}_{r} cos {θ}_{y} & cos {θ}_{r} sin {θ}_{p} cos {θ}_{y} + sin {θ}_{r} sin {θ}_{y} \\ sin {θ}_{r} cos {θ}_{p} & sin {θ}_{r} sin {θ}_{p} sin {θ}_{y} + cos {θ}_{r} cos {θ}_{y} & sin {θ}_{r} sin {θ}_{p} cos {θ}_{y} - cos {θ}_{r} sin {θ}_{y} \\ - sin {θ}_{p} & cos {θ}_{p} sin {θ}_{y} & cos {θ}_{p} cos {θ}_{y}\end{matrix} ]$$

Meanwhile, according to an embodiment, the first angle difference and the second angle difference may refer to differences in yaw value. In other words, in Equation 1 above, the relative angles θᵣ and θₚ may be 0°. Accordingly, the processor 1010 may calculate coordinate system transformation information based on Equation 2 and Equation 3 below by using the relative azimuth between the first robot base coordinate system (e.g., a camera robot base coordinate system) and the second robot base coordinate system (e.g., a surgical robot base coordinate system).

$${R}_{sRobot \to cRobot} = [ \begin{matrix}1 & 0 & 0 \\ 0 & cos {θ}_{sRobotTocRobot} & - sin {θ}_{sRobotTocRobot} \\ 0 & sin {θ}_{sRobotTocRobot} & cos {θ}_{sRobotTocRobot}\end{matrix} ] \qquad [ Equation 2 ]$$

$${R}_{cRobot \to sRobot} = [ \begin{matrix}1 & 0 & 0 \\ 0 & cos {θ}_{cRobotTosRobot} & - sin {θ}_{cRobotTosRobot} \\ 0 & sin {θ}_{cRobotTosRobot} & cos {θ}_{cRobotTosRobot}\end{matrix} ] \qquad [ Equation 3 ]$$

Here, R_{sRobot→cRobot} denotes rotation transformation from the surgical robot base coordinate system to the camera robot coordinate system, and R_{cRobot→sRobot} denotes rotation transformation from the camera robot base coordinate system to the surgical robot coordinate system.

In addition, θ_{sRobotTocRobot} may denote an azimuth of the camera robot base coordinate system (or the reference point of the camera robot base coordinate system) with respect to an axis of the surgical robot base coordinate system in a direction perpendicular to the ground, based on the surgical robot base coordinate system, and θ_{cRobotTosRobot} may denote an azimuth of the surgical robot base coordinate system (or the reference point of the surgical robot base coordinate system) with respect to an axis of the camera robot base coordinate system in a direction perpendicular to the ground, based on the camera robot base coordinate system.

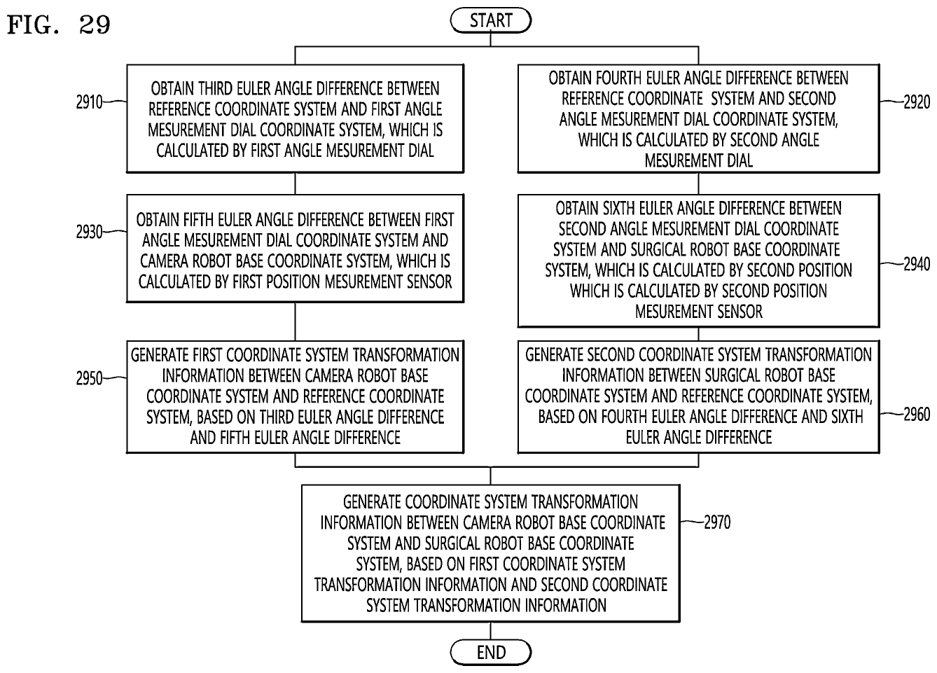

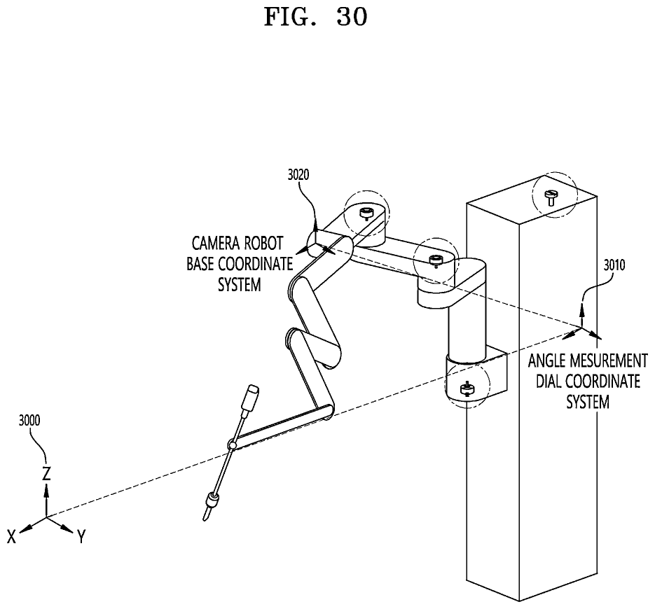

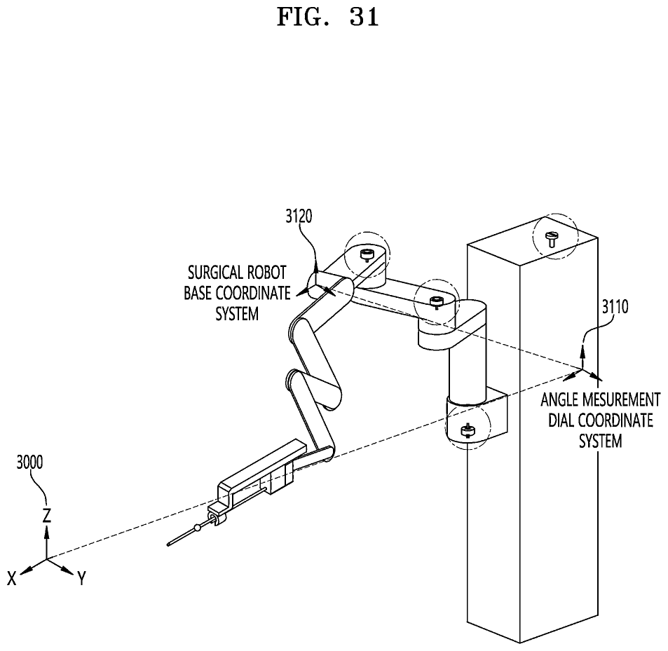

FIG. 29 is a flowchart for describing another example of a method of generating coordinate system transformation information in a robotic surgical system. FIGS. 30 and 31 are perspective views for describing another example of a coordinate system used in a robotic surgical system, according to an embodiment.

Referring to FIG. 29, in operation S2910, the processor 1010 may calculate a third Euler angle difference between the reference coordinate system and a first angle measurement dial coordinate system, based on a dial manipulation value obtained from a first angle measurement dial.

Thereafter, in operation S2920, the processor 1010 may calculate a fourth Euler angle difference between the reference coordinate system and a second angle measurement dial coordinate system, based on a dial manipulation value obtained from a second angle measurement dial.

The first angle measurement dial refers to an angle measurement dial mounted on a first robot included in a robotic surgical system, and the second angle measurement dial refers to an angle measurement dial mounted on a second robot. Hereinafter, for convenience of description, with reference to FIGS. 30 and 31, it is assumed that the first robot is a camera robot and the second robot is a surgical robot, but the present disclosure is not limited thereto.

Referring to FIG. 30, according to an embodiment, in a case in which the first angle measurement dial is mounted at a position of the camera robot other than a position that is a reference point of a camera robot base coordinate system 3020 from among positions of the camera robot at which the first angle measurement dial may be mounted, the first angle measurement dial may not use the camera robot base coordinate system 3020. In this case, the first angle measurement dial may use a first angle measurement dial coordinate system 3010.

For example, in a case in which the first angle measurement dial is not mounted on the camera robot at a connection point between a passive arm unit and an active arm unit included in the camera robot, which is the reference point of the camera robot base coordinate system 3020, the first angle measurement dial may use the first angle measurement dial coordinate system 3010 of which the reference point is the position of the camera robot at which the first angle measurement dial is mounted. As another example, in a case in which the first angle measurement dial is not mounted on the camera robot at a connection point between the passive arm unit and a body included in the camera robot, which is the reference point of the camera robot base coordinate system 3020, the first angle measurement dial may use the first angle measurement dial coordinate system 3010 of which the reference point is the position of the camera robot at which the first angle measurement dial is mounted.

Meanwhile, in a case in which the first angle measurement dial does not use the camera robot base coordinate system 3020, a dial manipulation value of a user obtained from the first angle measurement dial may correspond to an angle difference (a third angle difference) between a reference coordinate system 3000 and the first angle measurement dial coordinate system 3010. In other words, the processor 1010 may calculate the angle difference (the third angle difference) between the reference coordinate system 3000 and the first angle measurement dial coordinate system 3010, based on the dial manipulation value of the user obtained from the first angle measurement dial.

According to an embodiment, the processor 1010 may calculate a difference in yaw value between the preset reference coordinate system 3000 and the first angle measurement dial coordinate system 3010, excluding a difference in roll value and a difference in pitch value, based on a dial manipulation value of the user obtained from the angle measurement dial mounted on the camera robot. In other words, the third angle difference may refer to a difference in yaw value of the reference point of the first angle measurement dial coordinate system 3010 with respect to the reference coordinate system. For example, the third angle difference may refer to an azimuth θ_{cDial} of the first angle measurement dial coordinate system 3010 (or the reference point of the first angle measurement dial coordinate system 3010) with respect to an axis of the reference coordinate system in a direction perpendicular to the ground, based on the preset reference coordinate system 3000.

Referring to FIG. 31, according to an embodiment, in a case in which the second angle measurement dial is mounted at a position of the surgical robot other than a position that is a reference point of a surgical robot base coordinate system 3120 from among positions of the surgical robot at which the second angle measurement dial may be mounted, the second angle measurement dial may not use the surgical robot base coordinate system 3120. In this case, the second angle measurement dial may use a second angle measurement dial coordinate system 3110. Meanwhile, among descriptions to be provided below with reference to FIG. 31, redundant descriptions provided above with reference to FIG. 30 will be omitted.

Meanwhile, in a case in which the second angle measurement dial does not use the surgical robot base coordinate system 3120, a dial manipulation value of the user obtained from the second angle measurement dial may correspond to an angle difference (a fourth angle difference) between the reference coordinate system 3000 and the second angle measurement dial coordinate system 3110. In other words, the processor 1010 may calculate the angle difference (the fourth angle difference) between the reference coordinate system 3000 and the second angle measurement dial coordinate system 3110, based on the dial manipulation value of the user obtained from the second angle measurement dial.

According to an embodiment, the processor 1010 may calculate a difference in yaw value between the preset reference coordinate system 3000 and the second angle measurement dial coordinate system 3110, excluding a difference in roll value and a difference in pitch value, based on a dial manipulation value of the user obtained from the second angle measurement dial mounted on the surgical robot. In other words, the fourth angle difference may refer to a difference in yaw value of the reference point of the second angle measurement dial coordinate system 3110 with respect to the reference coordinate system. For example, the fourth angle difference may refer to an azimuth θ_{sDial} of the second angle measurement dial coordinate system 3110 (or the reference point of the second angle measurement dial coordinate system 3110) with respect to an axis of the reference coordinate system in a direction perpendicular to the ground, based on the preset reference coordinate system 3000.

Referring again to FIG. 29, in operation S2930, the processor 1010 may obtain a fifth angle difference between the first angle measurement dial coordinate system and a first robot base coordinate system, based on a first angle measurement value obtained from a first angle measurement sensor.

Thereafter, in operation S2940, the processor 1010 may calculate a sixth angle difference between the second angle measurement dial coordinate system and the second robot base coordinate system, based on a second angle measurement value obtained from the second angle measurement sensor. As in operations S2910 and S2920, for convenience of description, it is assumed that the first robot is a camera robot and the second robot is a surgical robot, but the present disclosure is not limited thereto.

Referring to FIG. 30, as described above regarding operation S2910, in a case in which the first angle measurement dial uses the first angle measurement dial coordinate system 3010 according to an embodiment, it is also necessary to generate coordinate system transformation information between the first angle measurement dial coordinate system 3010 and the camera robot base coordinate system 3020.

According to an embodiment, the first angle measurement sensor (not shown) mounted on a robotic arm unit of the first robot (e.g., a camera robot) may obtain a first angle measurement value between the first angle measurement dial coordinate system 3010 and the camera robot base coordinate system 3020. In addition, the processor 1010 may calculate a fifth angle difference based on the first angle measurement value obtained from the first angle measurement sensor.

Here, the first angle measurement sensor mounted on the robotic arm unit of the camera robot may refer to a plurality of sensors, and the first angle measurement value may be a result of performing an operation on angle measurement values measured respectively by the plurality of sensors. For example, in a case in which the robotic arm unit of the camera robot includes two or more passive arm units, at least one first angle measurement sensor may be mounted on the robotic arm unit of the camera robot. For example, referring to the camera robot illustrated in FIG. 30, in a case in which the robotic arm unit of the camera robot includes four passive arm units, three first angle measurement sensors may be mounted on the robotic arm unit of the camera robot. For example, each first angle measurement sensor may obtain an angle difference between two passive arm units, and the first angle measurement value may be a result of performing an operation on the angle measurement values obtained respectively by the angle measurement sensors. The processor 1010 may calculate an angle difference (the fifth angle difference) between the first angle measurement dial coordinate system and the camera robot base coordinate system, based on the first angle measurement value (a result of performing an operation on angle measurement values).

According to an embodiment, the first angle measurement sensor may measure a difference in yaw value between the first angle measurement dial coordinate system 3010 and the camera robot base coordinate system 3020, excluding a difference in roll value and a difference in pitch value. In other words, the processor 1010 may calculate a difference in yaw value of the camera robot base coordinate system 3020 (or the reference point of the camera robot base coordinate system 3020) with respect to the first angle measurement dial coordinate system 3010, based on the first angle measurement value obtained from the first angle measurement sensor. For example, the fifth angle difference may refer to an azimuth θ_{cDial→CRobot} of the camera robot base coordinate system 3020 with respect to an axis of the first angle measurement dial coordinate system 3010 in a direction perpendicular to the ground, based on the first angle measurement dial coordinate system 3010.

Hereinafter, description will be provided with reference to FIG. 31, but redundant descriptions provided above with reference to FIG. 30 will be omitted.

Referring to FIG. 31, according to an embodiment, a second angle measurement sensor (not shown) mounted on a robotic arm unit of a second robot (e.g., a surgical robot) may calculate an angle difference (a sixth angle difference) between the second angle measurement dial coordinate system 3110 and the surgical robot base coordinate system 3120. For example, in a case in which a robotic arm unit of a surgical robot includes four passive arm units, three second angle measurement sensors may be mounted on the robotic arm unit of the surgical robot. For example, each second angle measurement sensor may obtain an Euler angle difference between two passive arm units, and the second angle measurement value may be a result of performing an operation on the angle measurement values obtained respectively by the angle measurement sensors. The processor 1010 may calculate an angle difference (the sixth angle difference) between the second angle measurement dial coordinate system and the surgical robot base coordinate system, based on the second angle measurement value (a result of performing an operation on angle measurement values).

According to an embodiment, the second angle measurement sensor may measure a difference in yaw value between the second angle measurement dial coordinate system 3110 and the surgical robot base coordinate system 3120, excluding a difference in roll value and a difference in pitch value. In other words, the processor 1010 may calculate a difference in yaw value of the surgical robot base coordinate system 3120 (or the reference point of the surgical robot base coordinate system 3120) with respect to the second angle measurement dial coordinate system 3110, based on the second angle measurement value obtained from the second angle measurement sensor. For example, the sixth angle difference may refer to an azimuth θ_{sDial→sRobot} of the surgical robot base coordinate system 3120 with respect to an axis of the second angle measurement dial coordinate system 3110 in a direction perpendicular to the ground, based on the second angle measurement dial coordinate system 3110.

Referring again to FIG. 29, in operation S2950, the processor 1010 may generate first coordinate system transformation information between the first robot base coordinate system and the reference coordinate system, based on the third angle difference and the fifth angle difference. Hereinafter, in operations S2950 and S2960, for convenience of description, it is assumed that the first robot is a camera robot and the second robot is a surgical robot, and thus, the first robot base coordinate system may refer to a camera robot base coordinate system, and the second robot base coordinate system may refer to a surgical robot base coordinate system.

For example, processor 1010 may calculate a rotation transformation matrix based on an angle difference. According to an embodiment, the processor 1010 may calculate a rotation transformation matrix based on the angle differences obtained in operations S2910 to S2940.

According to an embodiment, based on the above-described azimuth θ_{cDial}, the processor 1010 may calculate a rotation transformation matrix from the preset reference coordinate system (e.g., a coordinate system used by a surgical bed) to the first angle measurement dial coordinate system, based on Equation 4 below. In addition, based on the above-described azimuth θ_{cDial→CRobot}, the processor 1010 may calculate a rotation transformation matrix from the first angle measurement dial coordinate system to the first robot base coordinate system (e.g., a camera robot base coordinate system), based on Equation 5 below.

$${R}_{Bed \to cDial} = [ \begin{matrix}1 & 0 & 0 \\ 0 & cos {θ}_{cDial} & - sin {θ}_{cDial} \\ 0 & sin {θ}_{cDial} & cos {θ}_{cDial}\end{matrix} ] \qquad [ Equation 4 ]$$

$${R}_{cDial \to cRobot} = [ \begin{matrix}1 & 0 & 0 \\ 0 & cos {θ}_{cDialToBase} & - sin {θ}_{cDialToBase} \\ 0 & sin {θ}_{cDialToBase} & cos {θ}_{cDialToBase}\end{matrix} ] \qquad [ Equation 5 ] ]$$

The processor 1010 may generate first coordinate system transformation information between the first robot base coordinate system and the reference coordinate system, based on a rotation transformation matrix calculated by using the third angle difference and a rotation transformation matrix calculated by using the fifth angle difference. For example, the processor 1010 may calculate a rotation transformation matrix from the reference coordinate system (e.g., a coordinate system used by a surgical bed) to the camera robot base coordinate system, based on Equation 6 below.

$${R}_{bed \to cRobot} = {R}_{bed \to cDial} * {R}_{cDial \to cRobot} = \qquad [ Equation 6 ]$$

$$[ \begin{matrix}1 & 0 & 0 \\ 0 & cos {θ}_{cDial} & - sin {θ}_{cDial} \\ 0 & sin {θ}_{cDial} & cos {θ}_{cDial}\end{matrix} ] * [ \begin{matrix}1 & 0 & 0 \\ 0 & cos {θ}_{cDialToBase} & - sin {θ}_{cDialToBase} \\ 0 & sin {θ}_{cDialToBase} & cos {θ}_{cDialToBase}\end{matrix} ]$$

Thereafter, in operation S2960, the processor 1010 may generate second coordinate system transformation information between the second robot base coordinate system and the reference coordinate system, based on the fourth angle difference and the sixth angle difference.

For example, processor 1010 may calculate a rotation transformation matrix based on an angle difference. According to an embodiment, the processor 1010 may calculate a rotation transformation matrix based on the angle differences obtained in operations S2910 to S2940.

According to an embodiment, based on the above-described azimuth θ_{sDial}, the processor 1010 may calculate a rotation transformation matrix from the preset reference coordinate system (e.g., a coordinate system used by a surgical bed) to the second angle measurement dial coordinate system, based on Equation 7 below. In addition, based on the above-described azimuth θ_{sDial}→sRobot, the processor 1010 may calculate a rotation transformation matrix from the second angle measurement dial coordinate system to the second robot base coordinate system (e.g., a surgical robot base coordinate system), based on Equation 8 below.

$${R}_{Bed \to sDial} = [ \begin{matrix}1 & 0 & 0 \\ 0 & cos {θ}_{sDial} & - sin {θ}_{sDial} \\ 0 & sin {θ}_{sDial} & cos {θ}_{sDial}\end{matrix} ] \qquad [ Equation 7 ]$$

$${R}_{sDial \to sRobot} = [ \begin{matrix}1 & 0 & 0 \\ 0 & cos {θ}_{sDialToBase} & - sin {θ}_{sDialToBase} \\ 0 & sin {θ}_{sDialToBase} & cos {θ}_{sDialToBase}\end{matrix} ] \qquad [ Equation 8 ]$$

The processor 1010 may generate second coordinate system transformation information between the second robot base coordinate system and the reference coordinate system, based on a rotation transformation matrix calculated by using the fourth angle difference and a rotation transformation matrix calculated by using the sixth angle difference. For example, the processor 1010 may calculate a rotation transformation matrix from the reference coordinate system (e.g., a coordinate system used by a surgical bed) to the surgical robot base coordinate system, based on Equation 9 below.

$${R}_{bed \to sRobot} = {R}_{bed \to sDial} * {R}_{sDial \to sRobot} = \qquad [ Equation 9 ]$$

$$[ \begin{matrix}1 & 0 & 0 \\ 0 & cos {θ}_{sDial} & - sin {θ}_{sDial} \\ 0 & sin {θ}_{sDial} & cos {θ}_{sDial}\end{matrix} ] * [ \begin{matrix}1 & 0 & 0 \\ 0 & cos {θ}_{sDialToBase} & - sin {θ}_{sDialToBase} \\ 0 & sin {θ}_{sDialToBase} & cos {θ}_{sDialToBase}\end{matrix} ]$$

Thereafter, in operation S2970, the processor 1010 may generate coordinate system transformation information between the first robot base coordinate system and the second robot base coordinate system, based on the first coordinate system transformation information and the second coordinate system transformation information.

As described above regarding operation S2630, the processor 1010 may generate coordinate system transformation information between the first robot base coordinate system and the second robot base coordinate system, based on the first angle difference and the second angle difference.

According to an embodiment, the processor 1010 may first generate coordinate system transformation information between the first robot base coordinate system and the reference coordinate system, and coordinate system transformation information between the second robot base coordinate system and the reference coordinate system, and generate coordinate system transformation information between the first robot base coordinate system and the second robot base coordinate system by using the generated coordinate system transformation information.

In more detail, the processor 1010 may generate the first coordinate system transformation information between the first robot base coordinate system and the reference coordinate system, based on the third angle difference and the fifth angle difference. In addition, the processor 1010 may generate the second coordinate system transformation information between the second robot base coordinate system and the reference coordinate system, based on the fourth angle difference and the sixth angle difference. In addition, the processor 1010 may generate coordinate system transformation information between the first robot base coordinate system and the second robot base coordinate system, based on the first coordinate system transformation information and the second coordinate system transformation information.

According to an embodiment, the coordinate system transformation information between the first robot base coordinate system and the second robot base coordinate system may refer to a rotation transformation matrix from the second robot base coordinate system to the first robot base coordinate system. For example, the processor 1010 may calculate a rotation transformation matrix from the second robot base coordinate system (e.g., a surgical robot base coordinate system) to the first robot base coordinate system (e.g., a camera robot base coordinate system) by using the first coordinate system transformation information and the second coordinate system transformation information, based on Equation 10 below.

$${R}_{sRobot \to cRobot} = {R}_{bed \to sRobot}^{T} * {R}_{bed \to cRobot} \qquad [ Equation 10 ]$$

Here, R_{bed→sRobot} corresponds to the rotation transformation matrix from the reference coordinate system (e.g., a coordinate system used by a surgical bed) to the surgical robot base coordinate system, which is calculated based on Equation 9, and R^{T}_{bed→sRobot} corresponds to a transposed matrix. In addition, R_{bed→cRobot} corresponds to the rotation transformation matrix from the reference coordinate system (e.g., a coordinate system used by a surgical bed) to the camera robot base coordinate system, which is calculated based on Equation 6.

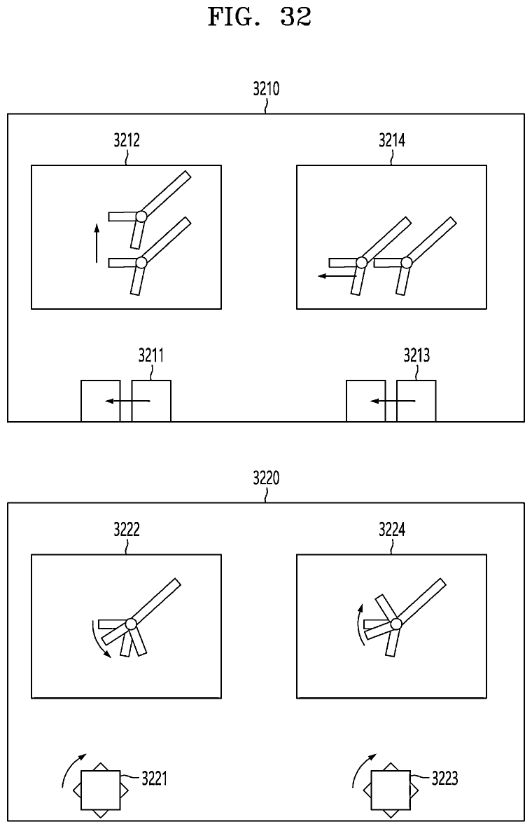

FIG. 32 is a diagram for describing an example of a robotic surgical system configured to operate based on coordinate system transformation information, according to an embodiment.

Referring to FIG. 32, in relation to manipulation 3210 and 3220 for moving a surgical instrument, movements of the surgical instrument corresponding to a case of moving a means that allows a user to manipulate the position and function of the surgical instrument in a vertical direction (3211 and 3213) and a case of rotating the means clockwise (3221 and 3223) may be displayed on the display member 10b.

For example, when the movement of the surgical instrument displayed on the display member 10b in correspondence to a case of moving the master robot 10 to the left (3211) is in the vertical direction (3212), it is difficult for the user to intuitively manipulate the surgical instrument.

On the contrary, according to the embodiments of the present disclosure described in detail above with reference to FIGS. 26 to 31, when coordinate system transformation information is generated, and a movement in a surgical robot base coordinate system is transformed into a movement in a camera robot base coordinate system by using the coordinate system transformation information, the movement of the surgical instrument displayed on the display member 10b in correspondence to a case of moving the master robot 10 to the left (3213) is in the left direction (3214). This allows the user to perform surgery with a surgical robot by more accurately reflecting intuitive manipulation by the user.

Similarly, a movement of the surgical instrument displayed on the display member 10b in correspondence to a case of rotating the master robot 10 clockwise (3221) may be in the counterclockwise direction (3222).

On the contrary, when a movement in a surgical robot base coordinate system is transformed into a movement in a camera robot base coordinate system by using coordinate system transformation information, a movement of the surgical instrument displayed on the display member 10b in correspondence to a case of rotating the master robot 10 clockwise (3223) may be in the clockwise direction (3224).

Meanwhile, the above-described method may be written as a computer-executable program, and may be implemented in a general-purpose digital computer that executes the program by using a computer-readable recording medium. In addition, the structure of the data used in the above-described method may be recorded in a computer-readable recording medium through various units. The computer-readable recording medium includes a storage medium, such as a magnetic storage medium (e.g., ROM, RAM, a universal serial bus (USB) drive, a floppy disk, or a hard disk) and an optically readable medium (e.g., a CD-ROM or a DVD).

The above-described method may be provided in a computer program product. The computer program product may be traded as commodities between sellers and buyers. The computer program product may be distributed in the form of a machine-readable storage medium (e.g., a CD-ROM), or may be distributed online (e.g., downloaded or uploaded) through an application store (e.g., Play Store™) or directly between two user devices. In a case of online distribution, at least a portion of the computer program product may be temporarily stored in a machine-readable storage medium such as a manufacturer's server, an application store's server, or a memory of a relay server.

It will be understood by those of skill in the art that the present disclosure may be implemented in a modified form without departing from the intrinsic characteristics of the descriptions provided above. Therefore, the disclosed methods should be considered in an illustrative rather than a restrictive sense, and the scope of the present disclosure should be defined by claims rather than the foregoing description, and should be construed to include all differences within the scope equivalent thereto.

1. A method, performed by a coordinate system transformation device in a robotic surgical system, of generating coordinate system transformation information, the method comprising:
calculating a first angle difference between a preset reference coordinate system and a first robot base coordinate system, based on a first dial manipulation value of a user obtained from a first angle measurement dial mounted on a first robot;
calculating a second angle difference between the preset reference coordinate system and a second robot base coordinate system, based on a second dial manipulation value of the user obtained from a second angle measurement dial mounted on a second robot; and
generating coordinate system transformation information between the first robot base coordinate system and the second robot base coordinate system, based on the first angle difference and the second angle difference.

2. The method of claim 1, wherein the first angle measurement dial or the second angle measurement dial is configured to receive physical dial manipulation by the user and obtain the first dial manipulation value corresponding to the first angle difference or the second dial manipulation value corresponding to the second angle difference.

3. The method of claim 1, wherein the calculating of the first angle difference comprises, in a case in which the first angle measurement dial does not use the first robot base coordinate system, calculating a third angle difference between the preset reference coordinate system and a coordinate system used by the first angle measurement dial, based on the first dial manipulation value obtained from the first angle measurement dial.

4. The method of claim 1, wherein the calculating of the second angle difference comprises, in a case in which the second angle measurement dial does not use the second robot base coordinate system, calculating a fourth angle difference between the preset reference coordinate system and a coordinate system used by the second angle measurement dial, based on the second dial manipulation value obtained from the second angle measurement dial.

5. The method of claim 3, wherein the first angle measurement dial is mounted at a position of the first robot other than a position that is defined as a reference point of the first robot base coordinate system.

6. The method of claim 4, wherein the second angle measurement dial is mounted at a position of the second robot other than a position that is defined as a reference point of the second robot base coordinate system.

7. The method of claim 5, wherein the coordinate system used by the first angle measurement dial has a reference point that is the position of the first robot at which the first angle measurement dial is mounted.

8. The method of claim 6, wherein the coordinate system used by the second angle measurement dial has a reference point that is the position of the second robot at which the second angle measurement dial is mounted.

9. The method of claim 3, further comprising calculating a fifth angle difference between a first angle measurement dial coordinate system and the first robot base coordinate system, based on a first angle measurement value obtained from a first angle measurement sensor mounted on a robotic arm of the first robot.

10. The method of claim 4, further comprising calculating a sixth angle difference between a second angle measurement dial coordinate system and the second robot base coordinate system, based on a second angle measurement value obtained from a second angle measurement sensor mounted on a robotic arm of the second robot.

11. The method of claim 1, wherein the generating of the coordinate system transformation information comprises:
generating first coordinate system transformation information between the first robot base coordinate system and the preset reference coordinate system by using the first angle difference;
generating second coordinate system transformation information between the second robot base coordinate system and the preset reference coordinate system by using the second angle difference; and
generating the coordinate system transformation information between the first robot base coordinate system and the second robot base coordinate system, based on the first coordinate system transformation information and the second coordinate system transformation information.

12. The method of claim 1, wherein the first angle difference or the second angle difference is a difference in yaw value of a reference point of the first robot base coordinate system with respect to the preset reference coordinate system, or a difference in yaw value of a reference point of the second robot base coordinate system with respect to the preset reference coordinate system.

13. A device for generating coordinate system transformation information in a robotic surgical system, the device comprising:
a non-transitory computer-readable medium configured to store at least one program; and
a processor configured to execute the at least one program to calculate a first angle difference between a preset reference coordinate system and a first robot base coordinate system, based on a dial manipulation value of a user obtained from a first angle measurement dial mounted on a first robot, calculate a second angle difference between the preset reference coordinate system and a second robot base coordinate system, based on a dial manipulation value of the user obtained from a second angle measurement dial mounted on a second robot, and generate coordinate system transformation information between the first robot base coordinate system and the second robot base coordinate system, based on the first angle difference and the second angle difference.

14. A non-transitory computer-readable recording medium having recorded thereon a program for executing the method of claim 1 on a computer.

15. A surgical robot comprising:
one or more robotic arms configured to perform a motion by handle manipulation by an operator;
a surgical instrument coupled to each of the one or more robotic arms;
an angle measurement dial configured to receive physical manipulation of a dial by a user to obtain a dial manipulation value of the user; and
a device to generate coordinate system transformation information based on an angle difference between coordinate systems,
wherein the device is to calculate a first angle difference between a preset reference coordinate system and a first robot base coordinate system, based on a dial manipulation value of the user obtained from a first angle measurement dial mounted on a first robot, calculate a second angle difference between the preset reference coordinate system and a second robot base coordinate system, based on a dial manipulation value of the user obtained from a second angle measurement dial mounted on a second robot, and generate coordinate system transformation information between the first robot base coordinate system and the second robot base coordinate system, based on the first angle difference and the second angle difference.

16. A laparoscopic camera robot comprising:
one or more robotic arms configured to perform a motion by handle manipulation by an operator;
a laparoscopic surgical camera coupled to each of the one or more robotic arms;
an angle measurement dial configured to receive physical manipulation of a dial by a user to obtain a dial manipulation value of the user; and
a device to generate coordinate system transformation information based on an angle difference between coordinate systems,
wherein the device is to calculate a first angle difference between a preset reference coordinate system and a first robot base coordinate system, based on a dial manipulation value of the user obtained from a first angle measurement dial mounted on a first robot, calculate a second angle difference between the preset reference coordinate system and a second robot base coordinate system, based on a dial manipulation value of the user obtained from a second angle measurement dial mounted on a second robot, and generate coordinate system transformation information between the first robot base coordinate system and the second robot base coordinate system, based on the first angle difference and the second angle difference.

17. A robotic surgical system comprising:
a surgical robot comprising a first angle measurement dial configured to receive a first physical manipulation of the first angle measurement dial by a user to obtain a first dial manipulation value of the user;
a laparoscopic camera robot comprising a second angle measurement dial configured to receive a second physical manipulation of the second angle measurement dial by the user to obtain a second dial manipulation value of the user; and
a master robot comprising a device to generate coordinate system transformation information based on an angle difference between coordinate systems,
wherein the device is to calculate a first angle difference between a preset reference coordinate system and a surgical robot base coordinate system, based on the first dial manipulation value of the user obtained from the first angle measurement dial mounted on the surgical robot, calculate a second angle difference between the preset reference coordinate system and a laparoscopic camera robot base coordinate system, based on the second dial manipulation value of the user obtained from the second angle measurement dial mounted on the laparoscopic camera robot, and generate coordinate system transformation information between the surgical robot base coordinate system and the laparoscopic camera robot base coordinate system, based on the first angle difference and the second angle difference.

18. The robotic surgical system of claim 17,
wherein the surgical robot has a sensor configured to measure a first angle measurement value between a first angle measurement dial coordinate system and the surgical robot base coordinate system.

19. The robotic surgical system of claim 17,
wherein the laparoscopic camera robot has a sensor configured to measure a second angle measurement value between a second angle measurement dial coordinate system and the laparoscopic camera robot base coordinate system.
